(19) United States
(12) Patent Application Publication
(10) Pub. No.: US 2024/0345086 A1
(43) Pub. Date: Oct. 17, 2024

(54) METHODS AND REAGENTS FOR THE DETECTION OF ANALYTES

(71) Applicant: UCL Business LTD, London (GB)

(72) Inventors: Kevin Mills, London (GB); Wendy Heywood, London (GB); Ivan Doykov, London (GB); James Moon, London (GB)

(21) Appl. No.: 18/579,635

(22) PCT Filed: Jul. 20, 2022
(86) PCT No.: PCT/GB2022/051875

§ 371 Date: Jan. 16, 2024

(30) Foreign Application Priority Data
Jul. 20, 2021 (GB) 2110404.7

(51) Int. Cl. G01N 33/569; G01N 33/68

(52) U.S. Cl. CPC G01N 33/56983; G01N 33/6848; G01N 2333/165; G01N 2469/20

(57) ABSTRACT

The present invention relates to a high throughput targeted proteomics-based method for identifying and quantifying one or more binding molecules in a biological fluid of an individual. The present invention involves the use of a plurality of bait molecules to capture the binding molecules. The invention uses proteomics techniques, such as mass spectrometry, for the identification and quantification of the binding molecules. The methods of the invention can be used to identify and quantify antibodies produced by an individual, and proteins expressed by an individual, following exposure to antigens, such as viral antigens, particularly coronavirus antigens and more particularly SARS-CoV-2. Exposure may be via vaccination or following natural infection. The methods of the invention can be used to identify and quantify molecules which bind to a range of proteins of interest, such as autoantigens and neoantigens, as well as to viral vectors.

### FIELD OF THE INVENTION

The present invention relates to a high throughput targeted proteomics-based method for identifying and quantifying one or more binding molecules in a biological fluid of an individual. The present invention involves the use of a plurality of bait molecules to capture the binding molecules. The invention uses proteomics techniques, such as mass spectrometry, for the identification and quantification of the binding molecules. The methods of the invention can be used to identify and quantify antibodies produced by an individual, and proteins expressed by an individual, following exposure to antigens, such as viral antigens, particularly coronavirus antigens and more particularly SARS-CoV-2. Exposure may be via vaccination or following natural infection. The methods of the invention can be used to identify and quantify molecules which bind to a range of proteins of interest, such as autoantigens and neoantigens, as well as to viral vectors.

### BACKGROUND TO THE INVENTION

With the acceleration of the worldwide severe acute respiratory syndrome coronavirus 2 (SARS-CoV-2) vaccination programme and with emerging variants threatening to undermine vaccine efficacy, it is becoming vital to develop efficient and faster methods to monitor and understand an individual's immune response to SARS-CoV-2. It is crucial to monitor this over time to determine when vaccine boosters may be needed and to determine responses to emerging variants.

A common strategy for monitoring the prevalence of SARS-CoV-2 in populations across the world is to determine the antibody response against SARS-CoV-2. Various immunoassays have been developed to determine an anti-SARS-CoV-2 antibody response in individuals. These tests include enzyme-linked immunosorbent assays (ELISA) which rely on the detection of antibodies generated against SARS-CoV-2 antigens.

Currently antibody responses are monitored by measurement of immunoglobulin G (IgG) typically by ELISA based methods. IgG consists of 4 subtypes (1-4) which all may or may not, contribute to immunity against SARS-CoV-2. Most current ELISA assays are IgG-based. They typically measure the total amount of IgG or the main subtype IgG1.

The Roche Elecsys® Anti-SARS-CoV-2 assay is currently considered the gold standard ELISA test for detecting and quantifying antibodies generated against SARS-CoV-2. This assay uses a recombinant protein representing the receptor binding domain (RBD) of the S (spike) antigen of SARS-CoV-2 in a double-antigen sandwich assay format. This assay favours detection of high affinity antibodies (total IgG) against the SARS-CoV-2 RBD and is intended as an aid to assess the adaptive humoral immune response to the SARS-CoV-2 S protein. It involves the measurement of electrochemilumenescence to quantify the amount of anti-SARS-CoV-2 antibody bound to the antigen. Thus, this assay uses a RBD (antigen) as a “bait” molecule to capture antibodies specifically generated against the RBD of SARS-CoV-2. The RBDs of this assay are modified with biotin and separately with ruthenyl which allows for voltaic measurements. A blood sample obtained from a patient is then incubated with RBD conjugated to biotin and RBD conjugated to ruthenyl. RBDs conjugated to biotin and those conjugated to ruthenyl will each bind to a different arm of an anti-RBD SARS-CoV-2 antibody. Magnetic microparticles coated with streptavidin are then added to the mixture containing the bait molecules and patient sample. The magnetic microparticles bind to biotin conjugated to an RBD allowing any RBD-complexes to be isolated and captured on an electrode. Electrochemiluminescence is then induced by applying a voltage to the isolate complexes and the voltage is measured with a photomultiplier. In such assay, the signal yield increases with the antibody titre.

Other tests for detecting and quantifying antibodies generated against SARS-CoV-2 are also known in the art. These include the “EDI Novel Coronavirus COVID-19” assay by Epitope Diagnostic, the “LIAISON SARS-CoV-2” assay by Diasorin, and the “SARS-CoV-2” assay by Euroimmun. Each of these assays are also ELISA-based. Each involves a different SARS-CoV-2 bait molecule selected from the nucleocapsid protein (N), S1 spike protein (S1), S2 spike protein (S2), and receptor binding domain protein (RBD) for the capture of antibodies specific to these antigens. These assays also rely on the detection of IgG antibodies.

Accordingly, current tests to detect a previous infection with SARS-CoV-2 in an individual are solely ELISA based. These tests rely on the detection of a patients' own antibodies that have been generated against SARS-CoV-2 virus antigens. They only indicate the presence of antibodies generated against a chosen antigen. They do not provide any information as to other molecules such as proteins which may form complexes with the antigen either by direct binding or indirect binding. ELISA based tests also require a kite marked kit made by a large manufacturer, may suffer reproducibility problems, are difficult to multiplex, and are not as accurate or specific as other analytical techniques. In addition, ELISA assays are not high-throughput. They are also labour intensive and can take up to a week to perform. Such tests are also relatively expensive. Therefore, these assays are not particularly suitable for rapid use on a population scale. Altogether, the disadvantages associated with current ELISA-based methods represents an obstacle for mass testing of individuals, e.g. to identify SARS CoV-2 infections worldwide.

As vaccine roll-out progresses, booster strategies are current being developed. Rapid mass testing to monitor SARS CoV-2-specific antibody levels could allow society to transition from a “booster for all” vaccination strategy to a selective booster vaccination strategy allowing a suitable booster to be administered to an individual based on past infections and/or vaccinations as determined by their antibody titres at a given timepoint. There is therefore a need for a test which would allow such mass testing in individuals to enable personalised vaccinations. Such a test would also help facilitate the determination of the timing of a booster vaccination for effective vaccination.

There is therefore an unmet need for a cost-effective, efficient, cheap, and high throughput test which can meet these needs.

Accordingly, the present invention relates to a method that allows for the detection, quantification and monitoring of one or more binding molecules in a sample obtained from a individual in a single assay. The present invention uses one or more bait molecule to capture one or more binding molecules that bind to the bait molecule. The captured binding molecules may be subjected to mass spectrometry for identification and quantification.

The method is amenable to the rapid detection, quantification and monitoring of proteins which bind to the spike, nucleocapsid or envelope proteins of SARS-COV-2, or fragments thereof, used as bait molecules, particularly immunoglobulins that have been generated against SARS-CoV-2 including IgM, IgA, IgG and subclasses thereof.

The present invention represents a new and alternative method to ELISA based methods for the detection and quantitation of proteins. The invention involves targeted proteomics. Targeted proteomics uses the highly accurate and specific technique of mass spectrometry to quantify proteins.

In particular, the use of targeted proteomic methods with triple quadrupole instruments allows the rapid translation of methods onto existing platforms already in place in most large hospitals in the UK. Triple quadrupole (e.g. LC-MS/MS) platforms are routinely used in clinical chemistry departments for measuring small molecules and for new-born screening of every child born in the UK. Thus the technology and expertise is already in place for the implementation of targeted protein assays via mass spectrometry platforms, making this an attractive approach for the purposes of mass screening in public health programs.

### SUMMARY OF THE INVENTION

The invention provides a method of identifying and quantifying one or more binding molecules in a biological fluid of an individual, wherein the binding molecules bind to a bait molecule, the method comprising:
a. providing a sample comprising biological fluid which has been taken from the individual;
b. capturing the one or more binding molecules by contacting the sample with a plurality of the bait molecules to form binding complexes comprising the bait molecules and the one or more binding molecules;
c. isolating the binding complexes; and
d. identifying and quantifying the one or more binding molecules in the biological fluid by analysing the captured binding molecules.

In any such method, following the step of isolating the binding complexes the complexes may be subjected to a digestion step to produce digestion products, and wherein the one or more binding molecules are identified and quantified by analysing the digestion products.

In any of the above-described methods, the one or more binding molecules may be proteins, wherein following isolation the complexes are subjected to proteolytic digestion to produce proteolytic digestion products, and wherein the one or more binding molecules are identified and quantified by analysing the proteolytic digestion products.

In any of the above-described methods, the step of analysing the captured binding molecules may be performed by mass spectrometry, preferably by liquid chromatography-tandem mass spectrometry (LC-MS/MS).

In any of the above-described methods, the bait molecules may be bound to a surface prior to contact with the sample, preferably wherein the plurality of bait molecules have been bound to the surface of a microplate. Alternatively, bait molecules may be bound to a surface during the process of isolating the binding complexes, preferably wherein the plurality of bait molecules are bound to the surface of a microparticle.

In any of the above-described methods, the bait molecule may comprise a pathogen polypeptide; an autoantigen polypeptide; a neoantigen polypeptide or a polypeptide of a gene therapy or vaccine delivery vector, such as a polypeptide of a viral vector e.g. an adenoviral vector, preferably AAV. The pathogen polypeptide may be a viral polypeptide. The viral polypeptide may be a coronavirus polypeptide, preferably wherein the coronavirus is severe acute respiratory syndrome coronavirus 2 (SARS-CoV-2). The viral polypeptide may comprise:
a. a full-length coronavirus spike protein, or a subunit or a fragment thereof,
b. a full-length coronavirus envelope protein or a fragment thereof,
c. a full-length coronavirus membrane protein or a fragment thereof, or
d. a full-length coronavirus nucleocapsid protein or a fragment thereof; and preferably wherein the coronavirus is severe acute respiratory syndrome coronavirus 2 (SARS-CoV-2).

The viral polypeptide may comprise:
a. an S1 subunit of a SARS-CoV-2 coronavirus spike protein or a fragment thereof, preferably wherein the S1 subunit comprises or consists of the amino acid sequence

(SEQ ID NO: 1)
QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVVL
SFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQ
DVNCTEVPVAIHADQLTPTWRVYSTGSNVFQTRAGCLIGAEHVNNSYECD
IPIGAGICASYQTQTNSPRRAR;

b. an S2 subunit of a SARS-CoV-2 coronavirus spike protein or a fragment thereof, preferably wherein the S2 subunit comprises or consists of the amino acid sequence

(SEQ ID NO: 2)
SVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQV
KQIYKTPPIKDFGGFNFSQILPDPSKPSKRSFIEDLLENKVTLADAGFIK
QYGDCLGDIAARDLICAQKFNGLTVLPPLLTDEMIAQYTSALLAGTITSG
WTFGAGAALQIPFAMQMAYRENGIGVTQNVLYENQKLIANQFNSAIGKIQ
DSLSSTASALGKLQDVVNQNAQALNTLVKQLSSNFGAISSVLNDILSRLD
KVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDR
LNEVAKNLNESLIDLQELGKYEQYIKWPWYIWLGFIAGLIAIVMVTIMLC
CMTSCCSCLKGCCSCGSCCKFDEDDSEPVLKGVKLHYT;

c. an angiotensin-converting enzyme 2 (ACE2) receptor binding domain (RBD) of an S1 subunit of SARS-CoV-2 coronavirus spike protein or a fragment thereof, preferably wherein the RBD of the S1 subunit comprises or consist of the amino acid sequence

(SEQ ID NO: 3)
RVQPTESIVRFPNITNLCPFGEVENATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGENCYFPLQSYGFQPTNGVGYQPYRVVVLSFELL
HAPATVCGPKKSTNLVKNKCVNF.

The plurality of bait molecules may comprise a subunit of a coronavirus protein, or a fragment of a coronavirus protein, and wherein the subunit or fragment is an immunogenic subunit or fragment.

The bait molecule may comprises a virion; preferably a virion that is not capable of replication, such as a pseudovirus. The virion may be a coronavirus virion, preferably a severe acute respiratory syndrome coronavirus 2 (SARS-CoV-2) virion.

In any of the above-described methods involving a viral polypeptide: (i) the viral polypeptide, (ii) the full-length coronavirus spike protein, subunit, RBD or any fragment thereof; (iii) the full-length coronavirus envelope protein or fragment thereof; (iv) the full-length coronavirus membrane protein or fragment thereof; (v) the full-length coronavirus nucleocapsid protein or fragment thereof; or (vi) the coronavirus virion may be from:
a. wild-type SARS-CoV-2; or
b. a variant of wild-type SARS-CoV-2, wherein the amino acid sequence of the variant differs from the amino acid sequence of wild-type by one or more amino acid mutations, optionally wherein one or more amino acid mutations are in an envelope protein or wherein one or more amino acid mutations are in a nucleocapsid protein, preferably wherein one or more amino acid mutations are in the spike protein.

In any of the above-described methods involving a viral polypeptide: (i) the viral polypeptide, (ii) the full-length coronavirus spike protein, subunit, RBD or any fragment thereof; (iii) the full-length coronavirus envelope protein or fragment thereof; (iv) the full-length coronavirus membrane protein or fragment thereof; (v) the full-length coronavirus nucleocapsid protein or fragment thereof; or (vi) the coronavirus virion may be from:
a. SARS-CoV-2 wild-type;
b. SARS-CoV-2 B.1.1.7 variant (alpha variant);
c. SARS-CoV-2 B.1.351 variant (beta variant);
d. SARS-CoV-2 P.1 variant (gamma variant);
e. SARS-CoV-2 B.1.617.2 variant (delta variant);
f. SARS-CoV-2 B.1.427/B.1.429 variant (epsilon variant);
g. SARS-CoV-2 P.2 variant (zeta variant);
h. SARS-CoV-2 B.1.525 variant (eta variant);
i. SARS-CoV-2 P.3 variant (theta variant);
j. SARS-CoV-2 B.1.526 variant (Iota variant);
k. SARS-CoV-2 B.1.617.1 variant (kappa variant);
l. SARS-CoV-2 C.37 variant (lambda variant); or
m. SARS-CoV-2 B.1.1.529 variant (omicron variant).

In any of the above-described methods, the one or more binding molecules which are identified and quantified are antibodies of IgG isotype, and wherein the antibodies are quantified according to concentration in the biological fluid of the individual.

In any of the above-described methods involving antibodies of IgG isotype, the method is capable of determining in the biological fluid of the individual an IgG antibody concentration which is statistically significantly increased compared to the IgG antibody concentration in a control, wherein the control is:
a) the average IgG antibody concentration in a population of individuals which have not been vaccinated against SARS-CoV-2 and which have not been infected with SARS-CoV-2; or
b) the average IgG antibody concentration in a population of individuals which have been vaccinated against SARS-CoV-2 and which have not been infected with SARS-CoV-2.

In any of the above-described methods involving antibodies of IgG isotype, the method may further comprise determining whether or not the IgG antibody concentration in the biological fluid of the individual is statistically significantly increased compared to the IgG antibody concentration in a control, wherein the control is the average IgG antibody concentration in a population of individuals which have not been vaccinated against SARS-CoV-2 and which have not been infected with SARS-CoV-2. Alternatively, the method may further comprise determining whether or not the IgG antibody concentration in the biological fluid of the individual is statistically significantly increased compared to the IgG antibody concentration in a control, wherein the control is the average IgG antibody concentration in a population of individuals which have been vaccinated against SARS-CoV-2 and which have not been infected with SARS-CoV-2.

In any of the above-described methods involving antibodies of IgG isotype, the one or more binding molecules which are identified and quantified may be antibodies of isotype IgG1, IgG2, IgG3 and/or IgG4, preferably IgG1.

In any of the above-described methods the one or more binding molecules which are identified and quantified are antibodies of IgA isotype and wherein the antibodies are quantified according to concentration in the biological fluid of the individual.

In any of the above-described methods involving antibodies of IgA isotype, the method may be capable of determining in the biological fluid of the individual an IgA antibody concentration which is statistically significantly increased compared to the IgA antibody concentration in a control, wherein the control is:
a. the average IgA antibody concentration in a population of individuals which have not been vaccinated against SARS-CoV-2 and which have not been infected with SARS-CoV-2; or
b. the average IgA antibody concentration in a population of individuals which have been vaccinated against SARS-CoV-2 and which have not been infected with SARS-CoV-2.

In any of the above-described methods involving antibodies of IgA isotype, the method may further comprise determining whether or not the IgA antibody concentration in the biological fluid of the individual is statistically significantly increased compared to the IgA antibody concentration in a control, wherein the control is the average IgA antibody concentration in a population of individuals which have not been vaccinated against SARS-CoV-2 and which have not been infected with SARS-CoV-2. Alternatively, the method may further comprise determining whether or not the IgA antibody concentration in the biological fluid of the individual is statistically significantly increased compared to the IgA antibody concentration in a control, wherein the control is the average IgA antibody concentration in a population of individuals which have been vaccinated against SARS-CoV-2 and which have not been infected with SARS-CoV-2.

In any of the above-described methods the one or more binding molecules which are identified and quantified are antibodies of IgM isotype and wherein the antibodies are quantified according to concentration in the biological fluid of the individual.

In any of the above-described methods involving antibodies of IgM isotype, the method may be capable of determining in the biological fluid of the individual an IgM antibody concentration which is statistically significantly increased compared to the IgM antibody concentration in a control, wherein the control is:
a. the average IgM antibody concentration in a population of individuals which have not been vaccinated against SARS-CoV-2 and which have not been infected with SARS-CoV-2; or
b. the average IgM antibody concentration in a population of individuals which have been vaccinated against SARS-CoV-2 and which have not been infected with SARS-CoV-2.

In any of the above-described methods involving antibodies of IgM isotype, the method may further comprise determining whether or not the IgM antibody concentration in the biological fluid of the individual is statistically significantly increased compared to the IgM antibody concentration in a control, wherein the control is the average IgM antibody concentration in a population of individuals which have not been vaccinated against SARS-CoV-2 and which have not been infected with SARS-CoV-2. Alternatively, the method may further comprise determining whether or not the IgM antibody concentration in the biological fluid of the individual is statistically significantly increased compared to the IgM antibody concentration in a control, wherein the control is the average IgM antibody concentration in a population of individuals which have been vaccinated against SARS-CoV-2 and which have not been infected with SARS-CoV-2.

In any of the above-described methods the binding molecules which are identified and quantified are complement C4b-binding protein (C4BP) and wherein the proteins are quantified according to their concentration in the biological fluid of the individual.

In any of the above-described methods involving complement C4b-binding protein (C4BP), the method may be capable of determining in the biological fluid of the individual a C4BP protein concentration which is statistically significantly increased compared to the C4BP protein concentration in a control, wherein the control is:
a. the average C4BP protein concentration in a population of individuals which have not been vaccinated against SARS-CoV-2 and which have not been infected with SARS-CoV-2; or
b. the average C4BP protein concentration in a population of individuals which have been vaccinated against SARS-CoV-2 and which have not been infected with SARS-CoV-2.

In any of the above-described methods involving complement C4b-binding protein (C4BP), the method may further comprise determining whether or not the C4BP protein concentration in the biological fluid of the individual is statistically significantly increased compared to the C4BP protein concentration in a control, wherein the control is the average C4BP protein concentration in a population of individuals which have not been vaccinated against SARS-CoV-2 and which have not been infected with SARS-CoV-2. Alternatively, the method may further comprise determining whether or not the C4BP protein concentration in the biological fluid of the individual is statistically significantly increased compared to the C4BP protein concentration in a control, wherein the control is the average C4BP protein concentration in a population of individuals which have been vaccinated against SARS-CoV-2 and which have not been infected with SARS-CoV-2.

In any of the above-described methods the binding molecules which are identified and quantified are complement component 4B (C4B) and wherein the proteins are quantified according to their concentration in the biological fluid of the individual.

In any of the above-described methods involving complement component 4B (C4B), the method be capable of determining in the biological fluid of the individual a C4B protein concentration which is statistically significantly increased compared to the C4B protein concentration in a control, wherein the control is:
a. the average C4B protein concentration in a population of individuals which have not been vaccinated against SARS-CoV-2 and have not been infected with SARS-CoV-2; or
b. the average C4B protein concentration in a population of individuals which have been vaccinated against SARS-CoV-2 and have not been infected with SARS-CoV-2.

In any of the above-described methods involving complement component 4B (C4B), the method may further comprise determining whether or not the C4B protein concentration in the biological fluid of the individual is statistically significantly increased compared to the C4B protein concentration in a control, wherein the control is the average C4B protein concentration in a population of individuals which have not been vaccinated against SARS-CoV-2 and which have not been infected with SARS-CoV-2. Alternatively, the method may further comprise determining whether or not the C4B protein concentration in the biological fluid of the individual is statistically significantly increased compared to the C4B protein concentration in a control, wherein the control is the average C4B protein concentration in a population of individuals which have been vaccinated against SARS-CoV-2 and which have not been infected with SARS-CoV-2.

In any of the above-described methods wherein the control is the average antibody concentration or protein concentration in a population of individuals which have not been vaccinated against SARS-CoV-2 and which have not been infected with SARS-CoV-2, when the antibody concentration or protein concentration is statistically significantly increased compared to the antibody concentration or protein concentration in the control, the individual may be classified as having produced antibodies to SARS-CoV-2 or produced proteins following exposure to SARS-CoV-2 antigens, and wherein the individual is thereby classified as having mounted an immune response to SARS-CoV-2.

In any of the above-described methods wherein the control is the average antibody concentration or protein concentration in a population of individuals which have not been vaccinated against SARS-CoV-2 and which have not been infected with SARS-CoV-2, when the antibody concentration or protein concentration is not statistically significantly increased compared to the antibody concentration or protein concentration in the control, the individual may be classified as requiring a prime dose of a vaccine against SARS-CoV-2 or a booster dose of a vaccine against SARS-CoV-2.

In any of the above-described methods wherein the control is the average antibody concentration or protein concentration in a population of individuals which have been vaccinated against SARS-CoV-2 and which have not been infected with SARS-CoV-2, when the antibody concentration or protein concentration is statistically significantly increased compared to the antibody concentration or protein concentration in the control, the individual may be predicted to have been infected with SARS-CoV-2.

In any of the above-described methods, the method may exhibit a sensitivity of 98.8% and a specificity of 100%.

In any of the methods of the invention, the plurality of bait molecules may comprise a gene therapy or vaccine delivery vector, such as a viral vector, e.g. an adenoviral vector, preferably AAV; preferably wherein the gene therapy or vaccine delivery vector is not capable of replication. The plurality of bait molecules may comprise a vaccine viral delivery vector. The vaccine viral delivery vector selected from the group consisting of:
a. a replication-deficient adenoviral vector;
b. a replication defective adenoviral vector of ChAdOx1;
c. a replication defective adenoviral vector of serotype 5;
d. a replication defective adenoviral vector of serotype 26; and
e. replication defective adenoviral vectors of serotypes 5 and 26.

In any of the methods of the invention, the biological fluid may be serum, plasma, whole blood, saliva, sputum, mucus or nasopharyngeal fluid, such as nasopharyngeal mucus.

In any of the methods of the invention, the sample may consist of biological fluid which has been taken from the individual and wherein the biological fluid in the sample has not been diluted.

In any of the methods of the invention, the sample may comprise biological fluid comprising blood obtained from a dried blood spot.

In any of the methods of the invention, the sample may comprise nasopharyngeal fluid, such as nasopharyngeal mucus, obtained from a nasopharyngeal swab.

In any of the methods of the invention, the sample may comprise biological fluid comprising saliva obtained from a home test kit.

The invention further provides a kit comprising:
a. one or more vials each comprising a plurality of identical isolated bait molecules for use in capturing one or more binding molecules from a sample comprising biological fluid which has been taken from an individual;
b. at least one microplate and/or one or more vials each comprising microparticles for use in isolating binding complexes between the bait molecules and the one or more binding molecules;
c. one or more vials each comprising a plurality of identical molecules for use as a reagent blank, optionally wherein at least one vial comprises molecules of bovine serum albumin or casein, preferably wherein at least one vial comprises molecules of myoglobin;
d. optionally one or more vials each comprising a dilution buffer for use in diluting biological fluid which has been taken from an individual, such as phosphate-buffered saline (PBS);
e. optionally or more vials comprising a wash buffer for use in washing binding complexes between the bait molecules and the one or more binding molecules, such as phosphate-buffered saline (PBS);
f. one or more vials comprising a surfactant for use in solubilising proteins;
g. one or more vials comprising a digestion buffer comprising an enzyme capable of cleaving proteins, preferably wherein the enzyme is a protease; and
h. one or more vials comprising a plurality of identical molecules for use as an internal calibration standard when performing quantification of a protein by mass spectrometry, preferably wherein the molecules are peptides.

In any of the kits of the invention the one or more vials each comprising a plurality of identical isolated bait molecules may comprise a pathogen polypeptide; an autoantigen polypeptide; a neoantigen polypeptide or a polypeptide of a gene therapy or vaccine delivery vector, such as a polypeptide of a viral vector e.g. an adenoviral vector, preferably AAV.

In any of the kits of the invention the one or more vials each comprising a plurality of identical isolated bait molecules may comprise a pathogen polypeptide, an autoantigen polypeptide, a neoantigen polypeptide, a polypeptide of a gene therapy or vaccine delivery vector, a virion, a gene therapy or vaccine delivery vector, or any viral polypeptide, subunit or any fragment thereof as described and defined herein.

In any of the kits of the invention the one or more vials comprising a surfactant for use in solubilising proteins may comprise a surfactant which is:
a. sodium 3-[(2-methyl-2-undecyl-1,3-dioxolan-4-yl)methoxy]-1-propanesulfonate,
b. 3-[N,N-Dimethyl(3-myristoylaminopropyl)ammonio]propanesulfonate (amidosulfobetaine-14) or
c. 3, 12-α-Dihydroxy-5β-cholan-24-oic acid monosodium salt (sodium deoxycholate).

In any of the kits of the invention the one or more vials comprising a digestion buffer comprising an enzyme capable of cleaving proteins may comprise a protease which is:
a. trypsin
b. chymotrypsin,
c. LysC,
d. LysN,
e. AspN,
f. GluC, or
g. ArgC.

In any of the kits of the invention the one or more vials comprising a plurality of identical molecules for use as an internal calibration standard may comprise a plurality of identical heavy labelled peptides, preferably wherein the peptides are labelled using ¹³C₆¹⁵N₂-lysine and ¹³C₆¹⁵N₄-arginine.

In any of the kits of the invention the plurality of identical heavy labelled peptides have an amino acid sequence which is identical to an amino sequence of a pathogen polypeptide, an autoantigen polypeptide, a neoantigen polypeptide, a polypeptide of a gene therapy or vaccine delivery vector, a virion, a gene therapy or vaccine delivery vector, or any viral polypeptide, subunit or any fragment thereof as described and defined herein.

In any of the kits of the invention the one or more vials may each comprise a plurality of identical monoclonal antibody species, wherein antibodies of a vial bind to an amino acid sequence which is common between two, more or all of:
a. wild-type SARS-CoV-2;
b. a variant of wild-type SARS-CoV-2, wherein the amino acid sequence of the variant differs from the amino acid sequence of wild-type by one or more amino acid mutations, optionally wherein one or more amino acid mutations are in an envelope protein or wherein one or more amino acid mutations are in a nucleocapsid protein, preferably wherein one or more amino acid mutations are in the spike protein;
c. SARS-CoV-2 B.1.1.7 variant (alpha variant);
d. SARS-CoV-2 B.1.351 variant (beta variant);
e. SARS-CoV-2 P.1 variant (gamma variant);
f. SARS-CoV-2 B.1.617.2 variant (delta variant);
g. SARS-CoV-2 B.1.427/B.1.429 variant (epsilon variant);
h. SARS-CoV-2 P.2 variant (zeta variant);
i. SARS-CoV-2 B.1.525 variant (eta variant);
j. SARS-CoV-2 P.3 variant (theta variant);
k. SARS-CoV-2 B.1.526 variant (Iota variant);
l. SARS-CoV-2 B.1.617.1 variant (kappa variant);
m. SARS-CoV-2 C.37 variant (lambda variant); and
n. SARS-CoV-2 B.1.1.529 variant (omicron variant).
wherein the amino acid sequence is a sequence of (i) the full-length coronavirus spike protein, subunit, RBD or any fragment thereof; (ii) the full-length coronavirus envelope protein or a fragment thereof; (iii) the full-length coronavirus membrane protein or a fragment thereof; or (iv) the full-length coronavirus nucleocapsid protein or a fragment thereof.

The invention further provides a method of obtaining a binding molecule profile of an individual by identifying and quantifying in a biological fluid of an individual one or more binding molecules that bind to a bait molecule, the method comprising:
a. providing a sample comprising biological fluid which has been taken from the individual;
b. capturing the one or more binding molecules by contacting the sample with a plurality of the bait molecules to form binding complexes comprising the bait molecules and the one or more binding molecules;
c. isolating the binding complexes; and
d. identifying and quantifying the one or more binding molecules in the biological fluid by analysing the captured binding molecules, thereby obtaining the binding molecule profile.

The binding molecule profile may be a viral immune polypeptide profile, preferably a coronavirus immune polypeptide profile. More preferably the profile may be a SARS CoV-2 immune polypeptide profile and wherein the bait molecule is any of the SARS CoV-2 viral polypeptides disclosed herein. Preferably the bait molecule is a full-length coronavirus spike protein, or an immunogenic subunit or a fragment thereof, more preferably the bait molecule is a polypeptide consisting of or comprising a SARS CoV-2 spike S1 subunit, including a full-length SARS CoV-2 spike S1 subunit, further any of the SARS CoV-2 spike S1 subunits disclosed herein.

Accordingly provided is a method of obtaining a SARS CoV-2 immune polypeptide profile of an individual by identifying and quantifying in a biological fluid of an individual one or more binding molecules that bind to a bait molecule, the method comprising:
a. providing a sample comprising biological fluid which has been taken from the individual;
b. capturing the one or more binding molecules by contacting the sample with a plurality of the bait molecules to form binding complexes comprising the bait molecule and the one or more binding molecules and wherein the bait molecule consists or comprises a S1 coronavirus spike protein, or a subunit or a fragment thereof;
c. isolating the binding complexes; and
d. identifying and quantifying the one or more binding molecules in the biological fluid by analysing the captured binding molecules, thereby obtaining the SARS CoV-2 immune polypeptide profile.

Preferably the bait molecule consists or comprises a full-length S1 coronavirus spike protein, including any of the full-length S1 coronavirus spike proteins described herein or any (immunogenic) subunit or fragment thereof.

Any of the described methods of obtaining a profile can be performed using any of the steps disclosed herein, as appropriate.

Following the step of isolating the binding complexes the complexes are preferably subjected to a digestion step to produce digestion products, and wherein the one or more binding molecules are identified and quantified by analysing the digestion products. The step of identifying and quantifying the digestion products of the one or more captured binding molecules is preferably performed by mass spectrometry, more preferably by liquid chromatography-tandem mass spectrometry (LC-MS/MS).

In any of the described methods of obtaining a profile, or in any of the described methods of identifying and quantifying one or more binding molecules in a biological fluid of an individual, bait molecules may have been bound to a surface prior to contact with the sample, preferably wherein the plurality of bait molecules have been bound to the surface of a microplate. Alternatively, bait molecules may be bound to a surface during the process of isolating the binding complexes, preferably wherein the plurality of bait molecules are bound to the surface of a microparticle.

In any of the described methods of obtaining a profile, or in any of the described methods of identifying and quantifying one or more binding molecules in a biological fluid of an individual, the one or more binding molecules are preferably quantified according to concentration in the biological fluid of the individual.

In any of the described methods of obtaining a profile or in any of the described methods of identifying and quantifying one or more binding molecules in a biological fluid of an individual, the one or more binding molecules which are identified and quantified comprise any one, any combination or all of the following proteins:
1. antibodies of IgG isotype, including IgG1, IgG2, IgG3 and/or IgG4, preferably IgG1;
2. antibodies of IgA isotype, preferably IgA1;
3. antibodies of IgM isotype;
4. complement component 4B (C4B);
5. complement component C9 (C9); and/or
6. complement C1q.

In methods involving quantification by mass spectrometry, quantification of antibodies of a particular isotype is preferably performed by detecting a conserved peptide sequence common to all species of that particular isotype, and wherein the peptide sequence does not occur in the antibody variable region. The method can be performed using conserved peptide sequences for the following antibody isotypes:
1. total IgG;
2. antibodies of IgG1 isotype;
3. antibodies of IgG2 isotype;
4. antibodies of IgG3 isotype;
5. antibodies of IgG4 isotype;
6. antibodies of IgA isotype; and
7. antibodies of IgM isotype;
   optionally using a peptide sequence corresponding to the relevant immunoglobulin as listed in Table 9 and defined by SEQ ID NO.

In methods involving quantification by mass spectrometry, quantification of proteins is preferably performed by detecting a conserved peptide sequences. The method can be performed using conserved peptide sequences for the following proteins:
1. complement component 4B (C4B);
2. complement component C9 (C9); and
3. complement C1q.
optionally using a peptide sequence corresponding to the relevant protein as listed in Table 9 and defined by SEQ ID NO.

Any of the methods disclosed herein, including methods of obtaining a profile, may be performed using a bait molecule of any first species. The method may be repeated, either in series or in parallel, using a bait molecule of any second species, which is different from the first, and wherein the method is performed on a different aliquot of the same sample from step (a) above. The method may be repeated, either in series or in parallel, three or more times, each replicated method using bait molecules which differ from those used in the other replicated methods, and wherein the methods are performed on different aliquots of the same sample from step (a) above. By performing the method multiple times using different bait molecules a comprehensive profile may be obtained for the individual providing information concerning the individual's status.

With regard to any of the methods for obtaining a SARS CoV-2 immune polypeptide profile, or in any of the described methods of identifying and quantifying one or more SARS CoV-2 binding molecules in a biological fluid of an individual the methods may be performed using a S1 coronavirus spike protein or a subunit or a fragment thereof from any first SARS CoV-2 strain/variant. The method may be repeated, either in series or in parallel, using a S1 coronavirus spike protein or a subunit or a fragment thereof from any other second SARS CoV-2 strain/variant, which is different from the first, and wherein the method is performed on a different aliquot of the same sample from step (a) above.

The method may be repeated, either in series or in parallel, three or more times, each replicated method using bait molecules which are a S1 coronavirus spike protein or a subunit or a fragment thereof from SARS CoV-2 strains/variants which differ from those used in the other replicated methods, and wherein the methods are performed on different aliquots of the same sample from step (a) above. By performing the method multiple times using S1 coronavirus spike proteins or subunits or fragments from different SARS CoV-2 strains/variants a comprehensive immune profile may be obtained for the individual providing information concerning the individuals immune status for any given strain/variant.

With regard to any of the methods disclosed herein for identifying and quantifying one or more binding molecules in a biological fluid of an individual, or for obtaining a profile, the step of quantifying in the biological fluid the one or more captured binding molecules comprises:
a) obtaining a preparation of a reference binding molecule having identifiable characteristics, wherein the identifiable characteristics are present in the bait molecules;
b) measuring the concentration of the reference binding molecules, e.g. in a mass spectrometer, to obtain standard abundance values;
c) standardising the one or more captured binding molecules to the reference binding molecules using the abundance values, optionally to obtain a ratio, wherein the one or more captured binding molecules are analysed by the same method used to measure the reference binding molecule.

Where the one or more captured binding molecules are proteins, the reference binding molecule may be a tryptic peptide having a specific peptide sequence as the identifiable characteristics, and wherein the specific peptide sequence is present in the bait molecules.

With regard to any of the methods disclosed herein for identifying and quantifying using mass spectrometry, e.g. LC-MS/MS, one or more SARS CoV-2 binding molecules in a biological fluid of an individual, or for any of the methods disclosed herein for obtaining a SARS CoV-2 immune polypeptide profile, the step of quantifying in the biological fluid the one or more captured binding molecules comprises:
a) obtaining a preparation of a SARS-CoV-2 spike protein tryptic peptide, preferably S1 spike, that has a peptide sequence that is conserved across all SARS-CoV-2 strains/variants and is present in the bait molecules;
b) measuring the concentration of the tryptic peptide in a mass spectrometer to obtain standard abundance values; and
c) standardising the digestion products of the one or more captured binding molecules to the tryptic peptide using the abundance values, optionally to obtain a ratio, wherein the digestion products of the one or more captured binding molecules are analysed by the same mass spectrometry method used to measure the tryptic peptide.

### BRIEF DESCRIPTION OF THE FIGURES

FIG. 1: shows a graphical representation of the method disclosed herein and a description of the use of SARS-CoV-2 S1 protein as a ‘bait molecule’ to attract antibodies, antibody-associated proteins and proteins with an affinity for SARS-CoV-2. Once captured from a biological matrix, these proteins are digested with trypsin into peptides and are then both identified and quantitated using targeted proteomics/mass spectrometry.

FIG. 2: A shows a standard curve for IgG1 r²=0.99. The highest positive value sample is indicated at 255.14 fmol. B Shows a box and whisker plot of the pre-2020 archive samples. A cut off value based on 3 standard deviations is indicated. A negative, low and high QC repeats are shown for comparison.

FIG. 3: shows Spearman correlation analysis between LC-MS/MS IgG1 levels against S1 protein for A Wuhan Hu-1 (GenBank: accession no. QHD43416.1), B alpha B.1.1.7 or C beta B.1.351, to Roche Elecsys® anti-SARS-CoV-2 ECLIA Spike RBD Ab titer. These data show IgG1 levels analysed using the multiplex-LC MS/MS assay and that there is a statistically significant correlation with the conventional Roche Elecsys® anti-SARS-CoV-2 ECLIA Spike assay. The results obtained for the Wuhan Hu-1, UK (alpha B.1.1.7) and South African (beta B.1.351) strains, were determined to be 0.798 (p<0.0001), 0.855 (p<0.0001) and 0.902 (p<0.0001), respectively. These results confirm an excellent strong correlation between the technologies.

FIG. 4: shows a comparison of standardised (Z score) Immunoglobulin and complement levels between S1 strain proteins in vaccinated individuals. A IgG1 B IgG2 C IgG3 D IgA E IgM. Comparison by Kruskal wallis post Dunn test

FIG. 5: shows a Spearman correlation analysis between nAb (IC50) titers against Wuhan Hu-1, alpha B.1.1.7 or beta B.1.351 S1 protein (labelled (i)-(ii) respectively) to A LC-MS/MS IgG1 levels; B LC-MS/MS total IgG (1-4); and C LC-MS/MS Complement C4.

FIG. 6: shows a Spearman correlation matrix of cellular response data from Reynold et al. compared with LC-MS/MS multiplex data.

FIG. 7: shows a comparison of standardised (Z score) complement C4 levels between strains in vaccinated individuals. Comparison was performed using a Kruskal wallis post Dunn test.

FIG. 8: shows IgG1 levels in dried blood spots and saliva spots over a 2 week period after vaccination.

FIG. 9: shows the principle of the targeted LC-MS/MS Immunocomplex assay. (A) shows a summarised workflow and schematical representation of the bait-capture-LCMS/MS assay. (B) Composition of the immunocomplex at different vaccination and pre-infection stages. Mean values used for each protein. (C) Heatmap of all proteins measured in the multiplex as determined by normalised mean values of the protein ratioed to spike peptide against each S1 bait variant and vaccination group MAC=membrane Attack Complex. Blue to Red colour scale indicates lowest to highest values.

FIG. 10: shows a comparison of LC-MS/MS IgG1 levels with other serology method and live viral neutralisation. (A) Roche Elecsys Anti-S assay vs IgG1 LC-MS/MS (B) Wuhan hu-1 neutralising antibodies vs Wuhan S1 IgG1 LC-MS/MS (C) Alpha neutralising antibodies vs alpha and Wuhan S1 IgG1 LC-MS/MS (D) Beta neutralising antibodies vs Beta and Wuhan S1 IgG1 LC-MS/MS (E) Delta neutralising antibodies vs delta and Wuhan S1 IgG1 LC-MS/MS. Significance determined by spearman correlation. n=141 in total for n=24 infection naïve group, n=23 previous infection group at pre, first and second vaccination time points.

FIG. 11: shows application of Immunocomplex assay to vaccinated participant cohort. (A) Shows the comparison of IgG1 levels binding to each VoC spike according to vaccine status in healthcare workers, who have had (+) or not had (−), a prior SARS CoV2 infection. Results are shown for the wild type Wuhan hu-1, Alpha, Beta and Delta spike variants. (B) summarises as an average percentage the IgG1 response to the VoC compared to Wuhan Hu-1. Comparison of other proteins of immunocomplex according to vaccine status for (C) IgM (D) IgG3 (E) IgA1 (F) complement C4 and (G) complement C9. Vaccination groups coloured according to exposure status. Green no exposure, yellow 1^{st} exposure (X1), orange 2nd exposure (X2) and red 3^{rd} exposure (X3). Data normalised for VoC comparison. Significance determined by non-parametric ANOVA and spearman correlation. Coloured significance bars indicate change due to pre-infection. n=24 infection naïve group, n=23 previous infection group.

FIG. 12: shows C1q response to variant S1 protein. (A) Shows the comparison of C1Q levels binding to each VoC spike according to vaccine status in healthcare workers, who have had (+) (n=23) or not had (−) (n=24), a prior SARS CoV2 infection. Results are shown for the wild type Wuhan hu-1, Alpha, Beta and Delta spike variants. Linear regression based on correlation showing changes in the slope/ratio for Delta for (B) C1Q vs IgG1 and no change in slope/ratio for Delta for (C) C1Q vs nAbs (log scale) analysis performed on all 4 vaccination groups combined n=94. (D) shows a summary of significant changes of the immunocomplex due to VoC compared to Wuhan-Hu-1 response. Blue decreased and red increased.

FIG. 13: shows response of triple vaccinated HCW to the Omicron BA.1 VoC. Comparison of triple vaccinated with or without prior infection against S1 protein from Wuhan hu-1 or Omicron BA.1 infection naïve n=25 (blue) prior infected n=36 (red) (A) IgG1 (B) IgA (C) C1q (1D) C4 (E) C9. (F) Comparison of ratio of C1q/IgG1 between VoC for double (n=47) and triple (n=76) vaccinated. Significance determined by non-parametric Kruskal Wallis test.

FIG. 14: shows Multivariate analysis of the immunocomplex response to SARS-CoV-2 infection or vaccine. A shows a multivariate OPLS-DA plot of all vaccine groups coloured according to their exposure to SARS CoV-2 vaccination and/or infection. The X0 (no exposure) and X1 (single exposure by infection or vaccination) groups (circled cluster), both cluster away from the doubly vaccinated or single vaccine+infection (X2) group and vaccinated plus previous SARS-CoV-2 infection groups (X3 exposure). X2 and X3 have a greater degree of variation indicating individuals have a large varied response to exposure. Pie chart (i) shows a typical profile based on mean values for the double vaccinated+pre-infection group with dominating IgG1. Pie chart (ii) shows a profile of an outlier with a profile dominated by IgG4 and IgG2 demonstrating individual heterogeneity in the immunocomplex. B shows the loading plot of the variables that drive the distribution of the groups in Figure S2A. These results indicate that IgG1 and C1Q are the variables that have the greatest influence and importance on separating the X2 and X3 each group from X0 and X1.

FIG. 15: shows Proof of principle of application of assay to other tissues. A demonstrates how the assay can be used to measure IgG1 levels in both saliva and saliva ‘lollipops’ 40-60 days after patients have tested positive for SARS COV2. These IgG1 levels are similar to those observed in individuals after receiving one vaccination or exposure (50% of patients have increased antibody levels). These results indicate that simple collection of saliva via absorbent filter paper used in the collection of bloodspots gives comparable results to neat saliva whilst simplifying the collection and storage of samples. B demonstrates how the assay has be used to measure IgG1 levels in the saliva of patients undergoing a full vaccination protocol. Saliva IgG1 levels were monitored in 3 patients over a 12-week period. As expected no increase in IgG1 levels were observed in the control patient who did not receive a vaccine but both patient 1 and 2 both demonstrated an increases in IgG1 after vaccination. Interestingly, patient 1 although demonstrated an increase in IgG1 levels after the first vaccination, no increases were observed after the second vaccination. In contrast, Patient 2 demonstrated a significant increase in IgG1 levels after both vaccinations. C shows the IgG1 levels in paired bloodspots and saliva measured daily, from the same patient for two weeks post vaccination. Although antibody levels are significantly lower in saliva than blood, relative antibody levels closely follow each other, indicating bloodspots and saliva ‘lollipops’ maybe be useful for high throughput, home testing of patients.

FIG. 16: spearman correlation matrix of components of the immunocomplex and corresponding neutralising antibodies. Total Ig is the sum of IgG 1-4 and total IC is sum of all the components of the immunocomplex assay.

FIG. 17: C1Q relationship to IgG1 and neutralising antibodies. (A) Shows correlation of IgG1 to nAbs for all vaccination groups and for variants together (B) Shows stronger correlation of C1Q to nAbs for all vaccination groups and for all variants together. Th ration of IgG1 to C1Q (C) shows an increased ratio in coloured according to exposure yellow first exposure, orange second exposure and red third exposure. Boxplots (ii) show min-max with mean +/−1SD. Significance determined by non-parametric ANOVA.

FIG. 18: shows response of triple vaccinated HCW to the Omicron BA.1 VoC. Comparison of triple vaccinated with or without prior infection against S1 protein from Wuhan hu-1 or Omicron BA.1 infection naïve n=25 (blue) prior infected n=36 (red) (A) IgM1 (B) IgG3 (C) IgG4. Significance determined by non parametric Kruskal Wallis test.

FIG. 19: shows skyline export of multiple transitions from method development of the peptides for IgG1, IgG2, IgG3 and IgG4.

FIG. 20: shows skyline export of multiple transitions from method development of the peptides for IgM, IgA1, C1QC and C9.

FIG. 21: shows an immunocomplex(IC) measured by targeted panel showing how the composition/profile of the spike bound (IC) proteins change with vaccination and infection. Triple vaccination increases the proportion of IgG4 antibodies and complement C9.

FIG. 22: shows A How the profile of your immunocomplex can be used to determine what prior infection you had. Stacked % charts for each spike experiment of triple vaccinated individuals showing profile change due to different variant infection compared to infection naïve. Boxed proteins are significantly different from naïve infection by non-parametric ANOVA p<0.05. Naïve n=25, alpha n=13, delta n=6, wuhan n=18, omicron n=8. B Summary table showing a profile signature for each infection. Red indicates greater and blue a lower proportion of protein bound to respective spike.

### DETAILED DESCRIPTION OF THE INVENTION

### Binding Molecules

A binding molecule is any molecule that binds to a bait molecule in any of the methods described or defined herein, either directly or indirectly.

A bait molecule is described and defined further herein.

A binding molecule may be any biological molecule that can be identified and quantified using the techniques described and defined herein.

A binding molecule may be an antibody, a polypeptide, an polynucleotide, a polyribonucleotide, a lipid, nucleic acids, ribonucleic acids, amino acids, carbohydrates, fatty acids, chemicals, drugs, vitamins, organic compounds, and/or inorganic compounds. A binding molecule is preferably a polypeptide or a protein or an antibody.

A binding molecule may be any molecule that is identified to be or known to be associated with an immune response, such as an immune response against a viral infection such as SARS-CoV-2. For example, a binding molecule may be an immune response associated protein, an anti-viral histone protein, a complement protein, a membrane attack complex protein, a soluble membrane attack complex protein, a platelet protein, or an immunoglobulin. More specifically, immune-response associated proteins includes Gelsolin (GSN), Transthyretin (TTHY), Heparin Cofactor 2 (SERPIND1), Serum Amyloid A4 (SAA4), Alpha-1-Antichymotrypsin (AACT), alpha-2-Macroglobulin (A2M), alpha-2-antiplasmin (A2AP), Angiotensinogen (AGT), Apoliporotein A1 (APOA1), Apoliporotein C3 (APOC3) and Inter-Alpha-Trypsin Inhibitor Heavy Chain 1 (ITIH1). Anti-viral histone proteins include Histone H2A (H2A) and Histone H2B (H2B). Complement protein includes Complement factor C4b (C4b), Complement factor C4 binding protein A (C4BPA) and Complement factor C4 binding protein B (CFBb). Membrane attack complex (MAC) proteins include Complement factor C5a (C5a), Complement factor C5b (C5b), Complement factor C8 (C8G) and Complement factor C9 (C9). Soluble membrane attack complex proteins (sMAC) include Clusterin (CLU), Paraoxonase 1 (PON1) and Vitronectin (VTN). A platelet protein may be Ras-Related Protein Rap-1A (RAP1AB). Immunoglobulins include IgG, IGHG1, IGHG2, IGHG3, IGHA1, IGHM, IGLC, IGLC, IgG1, IgG2, IgG3, IgG4, IgA, IgM, IgD and IgE.

Preferred binding molecules include IgG1, IgG2, IgG3, IgG4, IgA, IgM, complement C4b-binding protein (C4BP), complement component 4B (C4B), complement component 9 (C9) and complement C1q.

A list of further binding molecules is provided below.

| | UniProt |
|---|---|
| | accession |
| Protein | number |
| Complement component 4A (C4A) | P0C0L4 |
| C4b-binding protein alpha chain | P04003 |
| Complement C3 | P01024 |
| Complement C1q subcomponent subunit A | P02745 |
| Complement C1q subcomponent subunit C | P02747 |
| Complement C1q subcomponent subunit B | P02746 |
| Complement C5 | P01031 |
| Complement component C8 gamma chain | P07360 |
| Complement component C9 | P02748 |
| Immunoglobulin heavy constant gamma 1 | P01857 |
| Immunoglobulin heavy constant gamma 2 | P01859 |
| Immunoglobulin heavy constant gamma 3 | P01860 |
| Immunoglobulin heavy constant gamma 4 | P01861 |
| Immunoglobulin heavy constant alpha 1 | P01876 |
| Immunoglobulin heavy constant mu | P01871 |
| Angiotensin-converting enzyme 2 | Q9BYF1 |
| Ras-related protein Rap-1A | P62834 |
| Heparin cofactor 2 | P05546 |

The methods of the invention involve identifying and quantifying one or more binding molecules in a biological fluid of an individual, wherein the binding molecules bind to a bait molecule. The term “one binding molecule” is taken to mean a plurality of the same species of molecule. For example, in a situation where a bait molecule binds only a single species of binding molecule, the use of a plurality of identical bait molecules will mean that a plurality of binding molecules of the same species are captured. Bait molecules are also envisaged whereby a plurality of different species of molecule are captured. For example, when a coronavirus viral polypeptide is used as a bait, such as a spike protein, a plurality of different binding proteins will bind to the bait. Antibodies of IgG, IgA and IgM isotype will be expected to bind to a single coronavirus viral polypeptide used as a bait. In this situation all antibodies of IgG isotype can be regarded as “one binding molecule” within the meaning of the invention. Similarly all antibodies of IgA isotype can be regarded as “one binding molecule” and all antibodies of IgM isotype can be regarded as “one binding molecule”. Accordingly, when a coronavirus viral polypeptide is used as a bait it is possible to capture more than one “binding molecule”. The term “one binding molecule” can be used to describe a single species of binding molecule, e.g. a monoclonal antibody or a protein with a defined amino acid sequence such as complement C4b-binding protein (C4BP). Alternatively the term “one binding molecule” can be used to describe a generic group of binding molecule, such a mixed population of IgG antibodies.

### Sample Comprising Biological Fluid

A sample described herein may be any suitable sample comprising biological fluid.

For example, a sample may comprise a sample of a biological fluid taken from an individual, such as including but not limited to serum, plasma, whole blood, saliva, sputum, mucus, nasopharyngeal fluid, urine, mucous, vomit, faeces, and/or sweat. One example of a nsopharyngeal fluid is nasopharyngeal mucus. Other examples of biological fluids can be found here: https://en.wikipedia.org/wiki/Body fluid.

The sample comprising biological fluid may be or may not be diluted. If the sample is not diluted then the sample can itself be the biological fluid from the individual. Accordingly, in any of the methods described and defined herein the methods may comprise providing a sample consisting of biological fluid which has been taken from the individual. Alternatively, the if the sample has been diluted then the sample will comprise biological fluid from the individual. Accordingly, in any of the methods described and defined herein the methods may comprise providing a sample comprising biological fluid which has been taken from the individual.

If the biological fluid is diluted, any suitable dilution buffer may be used. A suitable dilution buffer may be phosphate-buffered saline (PBS). For example, if the biological fluid is serum, a suitable dilution buffer may be 0.05 mg/ml horse myoglobin solution in phosphate-buffered saline (PBS).

The biological fluid may be dried. The biological fluid may be reconstituted into a liquid having previously been dried.

The sample comprising biological fluid may comprise biological fluid obtained from a home test kit. For example, the sample comprising biological fluid may comprise blood obtained from a dried blood spot (DBS) e.g. obtained from a home test kit. The dried blood can then be taken up into a suitable dilution buffer prior to analysis. A suitable dilution buffer may be phosphate-buffered saline (PBS). A suitable dilution buffer may be 0.05 mg/ml horse myoglobin solution in PBS.

The sample comprising biological fluid may comprise saliva, sputum and/or nasopharyngeal fluid, e.g. obtained from a home test kit. For example, a nasopharyngeal swab or ‘lollipop’ such as a plastic stick or any other suitable means can be used to obtain a sample comprising saliva, sputum and/or nasopharyngeal fluid from an individual. Biological fluid from e.g. a nasopharyngeal swab can be taken up into a suitable dilution buffer prior to analysis. A suitable dilution buffer may be phosphate-buffered saline (PBS). A suitable dilution buffer may be 0.05 mg/ml horse myoglobin solution in PBS.

### Bait Molecules

The methods of the invention are capable of identifying and quantifying one or more binding molecules in a biological fluid of an individual, wherein the binding molecules bind to a bait molecule.

A bait molecule may be any molecule that is capable of capturing one or more binding molecules from a sample comprising biological fluid which has been taken from the individual and wherein the binding molecule can be identified and quantified using the techniques described and defined herein.

A bait molecule may be any biological molecule such as an antibody, a polypeptide, an polynucleotide, a polyribonucleotide, a lipid, nucleic acids, ribonucleic acids, amino acids, carbohydrates, fatty acids, chemicals, drugs, vitamins, organic compounds, and/or inorganic compounds. A bait molecule a preferably a peptide, polypeptide, or protein.

A bait molecule may be a molecule that comprises a pathogen polypeptide; an autoantigen polypeptide; a neoantigen polypeptide or a polypeptide of a gene therapy or vaccine delivery vector, such as a polypeptide of a viral vector.

### Bait Molecule—Pathogen Polypeptide

A bait molecule may comprise a pathogen polypeptide. A pathogen polypeptide may be a viral polypeptide. The viral polypeptide may be a coronavirus polypeptide or a polypeptide derived from a coronavirus polypeptide.

A viral polypeptide may comprise any part of a coronavirus polypeptide such as that derived from SARS-CoV-2.

A viral polypeptide may comprise a full-length coronavirus spike protein or a fragment thereof, a full-length coronavirus envelope protein or a fragment thereof, a full-length coronavirus membrane protein or a fragment thereof, and/or a full-length coronavirus nucleocapsid protein or a fragment thereof.

A viral polypeptide may comprise a polypeptide of SARS-Cov-2 spike (glyco)protein (S) or a fragment thereof. The SARS-Cov-2 spike (glyco)protein (S) may also be referred to as “spike protein”. It binds the cell-surface receptor to mediate virus entry. SARS-CoV-2 S protein is 1273 amino acid polypeptide. It consists of a signal peptide (amino acids 1-13) located at the N-terminus, an S1 subunit (14-685 residues), and an S2 subunit (686-1273 residues). The S1 and S2 subunits are responsible for receptor binding and membrane fusion, respectively. The S1 subunit comprises an N-terminal domain (14-305 residues) and a receptor-binding domain (RBD, 319-541 residues). The S2 subunit comprises a fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) (Xia S, Zhu Y, Liu M, Lan Q, Xu W, Wu Y, et al. Fusion mechanism of 2019-nCoV and fusion inhibitors targeting HR1 domain in spike protein. Cell Mol Immunol. 2020; 17:765-7). For example, the following subunits are derived from the original SARS-Cov-2 Wuhan strain which gave rise to the COVID-19 pandemic, as per accession number MN908947.3 (GenBank accession number for the spike protein is: QHD43416.1):

Full-length (amino acids 1-1273):
(SEQ ID NO: 4)
MFVFLVLLPLVSSQCVNLTTRTQLPPAYTNSFTRGVYYPDKVFRSSVLHSTQDLFLPFF
SNVTWFHAIHVSGTNGTKRFDNPVLPFNDGVYFASTEKSNIIRGWIFGTTLDSKTQSLL
IVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINIT
RFQTLLALHRSYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNATRFASVYAWN
RKRISNCVADYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKPFERDISTEI
YQAGSTPCNGVEGFNCYFPLQSYGFQPTNGVGYQPYRVVVLSFELLHAPATVCGPKKST
NLVKNKCVNFNFNGLTGTGVLTESNKKFLPFQQFGRDIADTTDAVRDPQTLEILDITPC
SFGGVSVITPGTNTSNQVAVLYQDVNCTEVPVAIHADQLTPTWRVYSTGSNVFQTRAGC
LIGAEHVNNSYECDIPIGAGICASYQTQTNSPRRARSVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSFIEDLLFNKV
TLADAGFIKQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQDSLSSTASA
LGKLQDVVNQNAQALNTLVKQLSSNFGAISSVLNDILSRLDKVEAEVQIDRLITGRLOS
LOTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTORNFYEPQIITTDNTF
VSGNCDVVIGIVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVMVTIMLCCMTS
CCSCLKGCCSCGSCCKFDEDDSEPVLKGVKLHYT
Signal peptide (amino acids 1-13):
(SEQ ID NO: 5)
MFVFLVLLPLVSS
S1 subunit (amino acids 14-685):
(SEQ ID NO: 1)
QCVNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKVC
EFQFCNDPFLGVYYHKNNKSWMESEFRVYSSANNCTFEYVSQPFLMDLEGKQGNFKNLR
EFVFKNIDGYFKIYSKHTPINLVRDLPQGFSALEPLVDLPIGINITRFQTLLALHRSYL
TPGDSSSGWTAGAAAYYVGYLQPRTFLLKYNENGTITDAVDCALDPLSETKCTLKSFTV
EKGIYQTSNFRVQPTESIVRFPNITNLCPFGEVENATRFASVYAWNRKRISNCVADYSV
LYNSASFSTFKCYGVSPTKLNDLCFTNVYADSFVIRGDEVRQIAPGQTGKIADYNYKLP
DDFTGCVIAWNSNNLDSKVGGNYNYLYRLFRKSNLKPFERDISTEIYQAGSTPCNGVEG
FNCYFPLQSYGFQPTNGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTN
TSNQVAVLYQDVNCTEVPVAIHADQLTPTWRVYSTGSNVFQTRAGCLIGAEHVNNSYEC
DIPIGAGICASYQTQTNSPRRAR
S2 subunit (amino acids 686-1273):
(SEQ ID NO: 2)
SVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQKFNGLTVLP
PLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVFVSN
GTHWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWPWYI
WLGFIAGLIAIVMVTIMLCCMTSCCSCLKGCCSCGSCCKFDEDDSEPVLKGVKLHYT
N-terminal domain (amino acids 14-305):
(SEQ ID NO: 6)
QCVNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKVC
EFQFCNDPFLGVYYHKNNKSWMESEFRVYSSANNCTFEYVSQPFLMDLEGKQGNFKNLR
EFVFKNIDGYFKIYSKHTPINLVRDLPQGFSALEPLVDLPIGINITRFQTLLALHRSYL
TPGDSSSGWTAGAAAYYVGYLQPRTFLLKYNENGTITDAVDCALDPLSETKCTLKS
Receptor-binding domain (RBD; amino
acids 319-541):
(SEQ ID NO: 3)
RVQPTESIVRFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGENCYFPLQSY
GFQPTNGVGYQPYRVVVLSFELLHAPATVCGPKKSTNLVKNKCVNF
Fusion peptide (FP) comprised within the
S2 domain (amino acids 788-806):
(SEQ ID NO: 7)
IYKTPPIKDFGGFNFSQIL
Heptapeptide repeat sequence 1 (HR1)
(amino acids 912-984):
(SEQ ID NO: 8)
TQNVLYENQKLIANQFNSAIGKIQDSLSSTASALGKLQDVVNQNAQALNTLVKQLSSNF
GAISSVLNDILSRL
HR2 (amino acids 1163-1213):
(SEQ ID NO: 9)
DVDLGDISGINASVVNIQKEIDRLNEVAKNLNESLIDLQELGKYEQYIKWP
TM domain (amino acids 1213-1237):
(SEQ ID NO: 10)
PWYIWLGFIAGLIAIVMVTIMLCCM
Cytoplasm domain (amino acids 1237-1273):
(SEQ ID NO: 11)
MTSCCSCLKGCCSCGSCCKFDEDDSEPVLKGVKLHYT

A viral polypeptide may comprise a full-length SARS-CoV-2 spike protein or a fragment thereof, an S1 subunit of a SARS-CoV-2 coronavirus spike protein or a fragment thereof, an S2 subunit of a SARS-CoV-2 coronavirus spike protein or a fragment thereof, and/or an ACE2 receptor binding domain (RBD) of an S1 subunit of SARS-CoV-2 coronavirus spike protein or a fragment thereof.

A SARS-CoV-2 spike protein may be monomeric, dimeric, trimeric or multimeric. Mature S glycoprotein on the viral surface is a heavily glycosylated trimer, each protomer of which is composed of 1260 amino acids (residues 14-1273). Accordingly, a SARS-CoV-2 spike protein may be glycosylated. A trimer of SARS-CoV-2 spike protein can be made in accordance with Li et al. (Viral Immunol. 2013 April; 26(2): 126-132) and Amanat et al. (Nat Med. 2020 July; 26(7): 1033-1036). A trimeric SARS-CoV-2 spike protein may comprise RBD subunits in an up (open) configuration or a closed (down) configuration Barnes et al. (Nature 588, 682-687 (2020)). In the down configuration, the spike protein is shielded from receptor binding (to ACE2) and in the up configuration, the spike protein is receptor-accessible. The generation of spike protein in up and down configurations is described in Henderson et al., (Nature Structural & Molecular Biology 27, 925-933 (2020)). In the methods of the present invention which involve the use of a coronavirus spike protein, the spike protein is preferably presented in the up (open) configuration.

A viral polypeptide may comprise SARS-CoV-2 spike protein comprising antigenic epitopes. An antigenic epitope is an epitope that is capable of generating an immune response when administered to an animal, preferably a human. An antigenic epitope may be comprised in the RBD, for example within positions 332-505 of the RBD. An antigenic epitope may comprise RBD residues involved in ACE2 binding such as any one or more amino acid positions of the RBD, as set out in Barnes et al. (Nature 588, 682-687 (2020)): K417, G446, Y449, Y453, L455, F456, A475, F486, N487, Y498, Q493, G446, Q448, T500, N501, G502 and/or Y505. An antigenic epitope may comprise RBD residues at any one or more of amino acid positions of the RBD: I332, T333, N334, C336, P337, F338, G339, F342, V341, N343, A344, T345, R346, Y351, N354, K356, R357, I358, S359, N360, C361, L368, Y369, N370, S371, A372, S373, F374, F377, K378, C379, Y380, G381, V382, S383, P384, T385, K386, L390, R403, D405, E406, R408, Q409, Q414, T415, G416, K417, D420, Y421, T430, G431, W436, S438, N439, N440, L441, D442, S443, K444, V445, G446, G447, N448, Y449, N450, L452, Y453, L455, F456, R457, K458, S459, N460, T470, 1472, Y473, Q474, A475, G476, S477, T478, N481, G482, V483, E484, G485, F486, N487, C488, Y489, F490, P491, L492, Q493, S494, Y495, G496, F497, Q498, P499, T500, N501, G502, V503, G504, Y505, F515, E516, and L517. Amino acid positions are in relation to wild-type RBD subunits as per the Wuhan-Hu-1 sequence of Genbank accession no. MN985325.1 as described in Barnes et al. (Nature 588, 682-687 (2020)). An antigenic epitope may comprise RBD residues at any one or more of the corresponding amino acids in any variant of SARS-CoV-2 as disclosed herein.

The coronavirus polypeptide may be a polypeptide of a SARS-CoV-2 variant of concern (VOC) or a SARS-CoV-2 variant of interest (VOI).

In any of the methods described herein which involve the detection and quantification of binding molecules which are captured by a SARS-CoV-2 spike protein which is used as the bait molecule, the bait molecule can be the full-length spike protein of any SARS-CoV-2 strain or variant described herein, or a (immunogenic) subunit or fragment thereof. The bait molecule can be the full-length S1 spike subunit of any SARS-CoV-2 strain or variant described herein, or a (immunogenic) further subunit or fragment thereof. Spike subunits, including the S1 spike subunit, are described above and can be identified by standard means, such as sequence alignment.

### SARS-CoV-2 Variants of Concern (VOC)

According to the World Health Organisation (https://www.who.int/en/activities/tracking-SARS-CoV-2-variants/) a SARS-CoV-2 variant of concern is a variant that meets the definition of a Variant of Interest (VOI) (see below) and, through a comparative assessment, has been demonstrated to be associated with one or more of the following changes at a degree of global public health significance:
increase in transmissibility or detrimental change in COVID-19 epidemiology; or
increase in virulence or change in clinical disease presentation; or
decrease in effectiveness of public health and social measures or available diagnostics, vaccines, therapeutics.

TABLE 1
designations of the different VOCs are outlined. Exemplary mutations are indicated for each VOC.

| | | | | | | | Key mutations |
|---|---|---|---|---|---|---|---|
| | | | | | | | compared with |
| | | | | Public | Earliest | | original Wuhan |
| WHO | Pango | GISAID | Nextstrain | Health | documented | Date of | strain (Pango A |
| label | lineage | clade/lineage | clade | England | samples | designation | lineage) |
| Alpha | B.1.1.7 | GRY (formerly | 20I/S:501Y.V1 | VOC-2020 Dec. 1 | United Kingdom, | 18 Dec. 2020 | with or without E484K* |
| | | GR/501Y.V1) | | | September 2020 | | aa:orf1ab:T1001I |
| | | | | | (British or | | aa:orf1ab:A1708D |
| | | | | | Kent variant) | | aa:orf1ab:I2230T |
| | | | | | | | del:11288:9 |
| | | | | | | | del:21765:6 |
| | | | | | | | del:21991:3 |
| | | | | | | | aa:S:N501Y |
| | | | | | | | aa:S:A570D |
| | | | | | | | aa:S:P681H |
| | | | | | | | aa:S:T716I |
| | | | | | | | aa:S:S982A |
| | | | | | | | aa:S:D1118H |
| | | | | | | | aa:Orf8:Q27* |
| | | | | | | | aa:Orf8:R52I |
| | | | | | | | aa:Orf8:Y73C |
| | | | | | | | aa:N:D3L |
| | | | | | | | aa:N:S235F |
| Beta | B.1.351 | GH/501Y.V2 | 20H/S:501Y.V2 | VOC-2020 Dec. 2 | South Africa, | 18 Dec. 2020 | aa:E:P71L |
| | | | | | May 2020 | | aa:N:T205I |
| | | | | | (South African | | aa:orf1a:K1655N |
| | | | | | variant) | | aa:S:D80A |
| | | | | | | | aa:S:D215G |
| | | | | | | | aa:S:K417N |
| | | | | | | | aa:S:A701V |
| | | | | | | | aa:S:N501Y |
| | | | | | | | aa:S:E484K |
| Gamma | P.1 | GR/501Y.V3 | 20J/S:501Y.V3 | VOC-2021 Jan. 2 | Brazil, | 11 Jan. 2021 | aa:orf1ab:S1188L |
| | | | | | November 2020 | | aa:orf1ab:K1795Q |
| | | | | | (Brazilian | | del:11288:9 |
| | | | | | variant) | | aa:S:L18F |
| | | | | | | | aa:S:T20N |
| | | | | | | | aa:S:P26S |
| | | | | | | | aa:S:D138Y |
| | | | | | | | aa:S:R190S |
| | | | | | | | aa:S:K417T |
| | | | | | | | aa:S:E484K |
| | | | | | | | aa:S:N501Y |
| | | | | | | | aa:S:H655Y |
| | | | | | | | aa:S:T1027I |
| | | | | | | | aa:orf3a:G174C |
| | | | | | | | aa:orf8:E92K |
| | | | | | | | aa:N:P80R |
| Delta | B.1.617.2 | G/452R.V3 | 21A/S:478K | VOC-2021 Apr. 2 | India, | VOI: 4 Apr. 2021 | S:T19R |
| | | | | | October 2020 | VOC: 11 May 2021 | S:L452R |
| | | | | | (Indian variant) | | S:T478K |
| | | | | | | | S:P681R |
| | | | | | | | S:D950N |
| | | | | | | | ORF3a:S26L |
| | | | | | | | M:I82T |
| | | | | | | | ORF7a:V82A |
| | | | | | | | ORF7a:T120I |
| | | | | | | | N:D63G |
| | | | | | | | N:R203M |
| | | | | | | | N:D377Y |

*-“B.1.1.7 with E484K” (VOC 2021 Feb. 2). “B.1.1.7 with E484K” is of the same lineage in the Pango nomenclature system, but has an additional E484K mutation.

### SARS-CoV-2 Variants of Interest (VOI)

According to the WHO, a SARS-CoV-2 isolate is a Variant of Interest (VOI) if, compared to a reference isolate, its genome has mutations with established or suspected phenotypic implications, and either:
has been identified to cause community transmission/multiple COVID-19 cases/clusters, or has been detected in multiple countries; or
is otherwise assessed to be a VOI by WHO in consultation with the WHO SARS-CoV-2 Virus Evolution Working Group.

TABLE 2
designations of the different VOIs are outlined. Exemplary mutations are indicated for each VOI.

| | | | | | | | Key mutations |
|---|---|---|---|---|---|---|---|
| | | | | | | | compared with |
| | | | | Public | Earliest | | original Wuhan |
| WHO | Pango | GISAID | Nextstrain | Health | documented | Date of | strain (Pango A |
| label | lineage | clade/lineage | clade | England | samples | designation | lineage) |
| Epsilon | B.1.427/ | GH/452R.V1 | 20C/S.452R | ? | United States | 5 Mar. 2021 | aa:S:S13I |
| | B.1.429 | | | | of America, | | aa:S:W152C |
| | | | | | March 2020 | | aa:S:L452R |
| Zeta | P.2 | GR | 20B/S.484K | VUI-2021 | Brazil, | 17 Mar. 2021 | Spike: E484K, |
| | | | | Jan. 1 | April 2020 | | (F565L*), D614G, |
| | | | | | | | V1176F |
| Eta | B.1.525 | G/484K.V3 | 20A/S484K | VUI-2021 | Multiple | 17 Mar. 2021 | aa:orf1ab:L4715F |
| | | | | Feb. 3 | countries, | | aa:S:Q52R |
| | | | | | December 2020 | | aa:S:E484K |
| | | | | | | | aa:S:Q677H |
| | | | | | | | aa:S:F888L |
| | | | | | | | aa:E:L21F |
| | | | | | | | aa:E:I82T |
| | | | | | | | del:11288:9 |
| | | | | | | | del:21765:6 |
| | | | | | | | del:28278:3 |
| Theta | P.3 | GR | 20B/S:265C | VUI-2021 | Philippines, | 24 Mar. 2021 | Spike: E484K, |
| | | | | Mar. 2 | January 2021 | | N501Y, and |
| | | | | | | | P681H |
| Iota | B.1.526 | GH | 20C/S:484K | ? | United States | 24 Mar. 2021 | Spike: (L5F*), |
| | | | | | of America, | | T95I, D253G, |
| | | | | | November 2020 | | (S477N*), (E484K*), |
| | | | | | | | D614G, (A701V*) |
| Kappa | B.1.617.1 | G/452R.V3 | 21A/S:154K | VUI-2021 | India, | 4 Apr. 2021 | Spike: (T95I), |
| | | | | Apr. 1 | October 2020 | | G142D, E154K, |
| | | | | | | | L452R, E484Q, |
| | | | | | | | D614G, P681R, |
| | | | | | | | Q1071H |
| Lambda | C.37 | GR/452Q.V1 | 20D | ? | Peru, | 14 Jun. 2021 | G75V, T76I, |
| | | | | | August 2020 | | L452Q, F490S, |
| | | | | | | | T859N |
| Omicron | B.1.1.529 | GRA | 21 and 22 | | South Africa | 26 Nov. 2021 | nsp3:K38R |
| | | (B.1.1.529 + | | | November 2021 | | nsp3:V1069I |
| | | BA.*) | | | | | nsp3:Δ1265 |
| | | | | | | | nsp3:L1266I |
| | | | | | | | nsp3:A1892T |
| | | | | | | | nsp4:T492I |
| | | | | | | | nsp5:P132H |
| | | | | | | | nsp6:Δ105-107 |
| | | | | | | | nsp6:A189V |
| | | | | | | | nsp12:P323L |
| | | | | | | | nsp14:I42V |
| | | | | | | | Spike A67V |
| | | | | | | | Δ69-70 |
| | | | | | | | T95I |
| | | | | | | | G142D, |
| | | | | | | | Δ143-145 |
| | | | | | | | Δ211 |
| | | | | | | | L212I |
| | | | | | | | ins214EPE |
| | | | | | | | G339D |
| | | | | | | | S371L |
| | | | | | | | S373P |
| | | | | | | | S375F |
| | | | | | | | K417N |
| | | | | | | | N440K |
| | | | | | | | G446S |
| | | | | | | | S477N |
| | | | | | | | T478K |
| | | | | | | | E484A |
| | | | | | | | Q493R |
| | | | | | | | G496S |
| | | | | | | | Q498R |
| | | | | | | | N501Y |
| | | | | | | | Y505H |
| | | | | | | | T547K |
| | | | | | | | D614G |
| | | | | | | | H655Y |
| | | | | | | | N679K |
| | | | | | | | P681H |
| | | | | | | | N764K |
| | | | | | | | D796Y |
| | | | | | | | N856K |
| | | | | | | | Q954H |
| | | | | | | | N969K |
| | | | | | | | L981F |
| | | | | | | | E T9I |
| | | | | | | | M D3G |
| | | | | | | | Q19E |
| | | | | | | | A63T |
| | | | | | | | N P13L |
| | | | | | | | Δ31-33 |
| | | | | | | | R203K |
| | | | | | | | G204R |

*= detected in some sequences but not all.

Specific bait molecules that can be used in the present invention are listed below:

SARS-CoV-2 Nucleocapsid protein Catalog No: Z03488-1 accession no. QHD43416.1 Ser2-Ala419);
Wuhan strain: SARS-CoV-2 Spike protein (S1) Catalog No: Z03501-1-accession no. QHD43416.1 (Gln14-Arg685);
Kent strain: Cat #: 40591-V08H7 Description: SARS-CoV-2 (2019-nCoV) Spike S1(HV69-70 deletion, N501Y, D614G)-His Recombinant Protein; and South Africa strain: Catalog No: Z03531-1; Name: SARS-CoV-2 Spike protein (S1, E484K, K417N, N501Y, His Tag) accession no. QHD43416.1.
Omicron (B.1.1.529/Omicron) Spike Glycoprotein (S1), Sheep Fc-Tag (HEK293) SARS-CoV-2 (B.1.1.529/Omicron) Spike subunit 1 protein. Relative to Wuhan-Hu-1 this protein has following amino acid changes: A67V, H69del, V70del, T95I, G142D, V143del, Y144del, Y145del, N211del, L212I, ins214EPE, G339D, S371L, S373P, S375F, K417N, N440K, G446S, S477N, T478K, E484A, Q493R, G496S, Q498R, N501Y, Y505H, T547K, D614G, H655Y.

A bait molecule described herein may comprise a polypeptide of a SARS-CoV-2 Variant of concern or a fragment thereof. A bait molecule described herein may comprise a polypeptide of a SARS-CoV-2 Variant of interest or a fragment thereof. A bait molecule may have one or more mutations compared to the original Wuhan strain (accession number for the genome: MN908947.3, GenBank accession number for the spike protein: QHD43416.1), also referred to as Wuhan hu-1 strain or wild-type strain in the present disclosure. For example, a bait molecule may one or more notable missense mutations found in a SARS-CoV-2 spike protein as outlined in Tables 1 and 2.

A viral polypeptide may comprise a full-length SARS-CoV-2 nucleocapsid protein (N) or a fragment thereof. The N protein is responsible for genome packaging. The N protein contains two structural domains: the N-terminal domain (NTD; residues 45 to 181) and the C-terminal dimerization domain (CTD; residues 248 to 365) flanked by long stretches of disordered regions accounting for almost half of the entire sequence. For example, in accordance with NCBI reference number: YP_009724397.2 the sequence for SARS-CoV-2 N protein is as follows:

Full-length sequence of the N protein
(amino acids 1-419):
(SEQ ID NO: 12)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQROKKOQTVTLLPAADL
DDFSKQLQQSMSSADSTQA
N-terminal domain (amino acids 45 to 181):
(SEQ ID NO: 13)
LPNNTASWFTALTQHGKEDLKFPRGQGVPINTNSSPDDQIGYYRRATRRI
RGGDGKMKDLSPRWYFYYLGTGPEAGLPYGANKDGIIWVATEGALNTPKD
HIGTRNPANNAAIVLQLPQGTTLPKGFYAEGSRGGSQ
C-terminal dimerization domain
(amino acids 248 to 365):
(SEQ ID NO: 14)
KKSAAEASKKPRQKRTATKAYNVTQAFGRRGPEQTQGNFGDQELIRQGTD
YKHWPQIAQFAPSASAFFGMSRIGMEVTPSGTWLTYTGAIKLDDKDPNFK
DQVILLNKHIDAYKTFPP

A viral polypeptide may comprise a full-length SARS-CoV-2 envelope protein (E) or a fragment thereof. The E protein forms a cation-selective channel across the ERGIC membrane. In SARS-CoV-1, E mediates the budding and release of progeny viruses. The E protein is a 75-residue viroporin with a N-terminal domain (residues 1-7), a transmembrane (TM) domain (residues 8-38) and a C-terminal domain (residues 39-75). For example, in accordance with NCBI reference number: YP_009724392.1 the sequence for SARS-CoV-2 E protein is as follows:

Full-length E protein (amino acids 1-75):
(SEQ ID NO: 15)
MYSFVSEETGTLIVNSVLLFLAFVVFLLVTLAILTALRLCAYCCNIVNVS
LVKPSFYVYSRVKNLNSSRVPDLLV
N-terminal domain (amino acids 1-7):
(SEQ ID NO: 16)
MYSFVSE
Transmembrane (TM) domain (amino acids 8-38):
(SEQ ID NO: 17)
ETGTLIVNSVLLFLAFVVFLLVTLAILTALR
C-terminal domain (amino acids 39-75):
(SEQ ID NO: 18)
LCAYCCNIVNVSLVKPSFYVYSRVKNLNSSRVPDLLV

### Bait Molecule—Autoantigen Polypeptide

A bait molecule may be an autoantigen polypeptide or a fragment thereof. An autoantigen polypeptide may be a polypeptide which is recognised by the immune system of an individual thus resulting in specific auto-immune disorders. Examples of auto-immune disorders include rheumatoid arthritis, systemic lupus erythematosus (SLE) or Lupus, scieroderma, polymyositis, inflammatory bowel disease, dermatomyositis, ulcerative colitis, Crohn's disease, vasculitis, psoriatic arthritis. Reiter's syndrome, exfoliative psoriatic dermatitis, pemphigus vulgaris, Sjorgren's syndrome, autoimmune uveitis, glomerulonephritis, post myocardial infarction cardiotomy syndrome, pulmonary hemosiderosis, amyloidosis, sarcoidosis, aphthous stomatitis, and other immune related diseases as presented herein and known in the related art.

Accordingly, an autoantigen polypeptide may be used as a bait molecule in the methods described and defined herein. Binding molecules which are captured by the autoantigen polypeptide can be identified and quantified using the methods described herein from biological fluid which has been taken from an individual suspected of having an autoimmune disorder in which the autoantigen polypeptide is implicated.

The identification and quantification of molecules which bind to the autoantigen polypeptide may provide useful information concerning the individual's status with regard to the autoimmune disorder, such as whether the individual can be diagnosed as having the autoimmune disorder or whether the individual is at risk of developing the autoimmune disorder.

### Bait Molecule—Neoantigen

A bait molecule may be a neoantigen polypeptide or a fragment thereof. A neoantigen polypeptide is a polypeptide not previously recognised by the immune system of an individual. A neoantigen polypeptide may be expressed on the surface of a cancer cell for example. Neoantigen polypeptides can arise through a variety of mutational events, with the numbers and types of mutation varying by cancer type. These events include point mutations, insertions or deletions (indels) and gene fusions. For example, neoantigens can arise from altered tumor proteins formed as a result of tumor mutations or from viral proteins.

Accordingly, a neoantigen or polypeptide comprising a neoantigen may be used as a bait molecule in the methods described and defined herein. Binding molecules which are captured by the neoantigen or polypeptide comprising the neoantigen can be identified and quantified using the methods described herein from biological fluid which has been taken from an individual suspected of having a disorder in which the neoantigen is implicated. The identification and quantification of molecules which bind to the neoantigen or polypeptide comprising the neoantigen may provide useful information concerning the individual's status with regard to the disorder, such as whether the individual can be diagnosed as having the disorder or whether the individual is at risk of developing the disorder.

### Bait Molecule—Vaccine or Gene Therapy Vector

A bait molecule may be an entire vaccine or gene therapy vector.

Alternatively, a bait molecule may be a polypeptide of a vaccine or gene therapy vector or a fragment thereof. For example, it may be a polypeptide of a viral vector e.g. an adenoviral vector, preferably AAV (adeno-associated vectors). Examples of viral vectors include retroviruses, lentiviruses, herpes simplex viruses, adenoviruses, and AAVs. AAV serotypes include for example, AAV1, AAV2, AAV3, AAV4, AAV5. AAV6, AAV7, AAV8, AAV9, AAV10, AAV11, AAV12. Gene therapy vectors include any vector used in gene therapy such as adenovirus vectors. These can be replication-defective or replication-competent. Replication-defective vectors may have the essential E1A and E1B genes deleted and replaced by an expression cassette with a high activity promoter such as the cytomegalovirus immediate early (CMV) promoter which drives expression of the foreign transgene. The immediate early E1A-coded proteins are essential for adenovirus replication; they induce expression of the delayed early genes in the E1B, E2, E3, and E4 transcription units and alter the expression of a multitude of cellular genes in order to facilitate adenvirus replication. E1B proteins in general inhibit the host cell apoptotic response to adenovirus infection. Adenvirus vectors may lack the E3 genes which in general prevent infected cells from elimination by the immune system and are not essential for adenovirus replication. E1A and E1B deleted adenovirus vectors are usually constructed from plasmids or adenovirus DNA containing the genetically modified adenvirus genome. These vectors may be produced in cell lines such as HEK293, PER.C6, or N52.E6 which retain and express the E1A and E1B genes. Vectors deleted for E1A, E1B, and E3 can exhibit leaky expression of other early as well as late adenvirus genes, and therefore transduced cells in animal models can be eliminated by a T cell response. Many replication-deficient vectors may therefore also lack the E4 region, which encodes genes for double strand DNA repair as well as other essential functions. E1A, E1B, E3, and E4 deleted vectors exhibit less leaky expression. Accordingly, complementing cell lines for these vectors express E1A, E1B, and E4 proteins.

Another replication-deficient adenovirus vector is a “helper-dependent” (HDAd) vector which has most of the genome deleted but retains the origins of DNA replication at each end of the genome as well as ˜500 base pairs at the left end of the genome that are required to package the genome into virions. These vectors have high cloning capacity and as such can retain multiple transgenes. HDAd vectors are constructed and propagated in the presence of a replication-competent helper adenovirus which provides the required early and late proteins necessary for replication. The helper adenvirus may have loxP sites flanking the packaging signal in the genome. The cell lines used to produce HDAd vectors may conditionally express Cre recombinase which excises the loxP-flanked packaging signal from the helper adenovirus genome thereby allowing for preferential packaging of the HDAd genome (which lacks the loxP sites).

A bait molecule may be a vaccine viral vectors such as those used current vaccines. For example vaccine viral vectors may be the modified chimpanzee adenovirus, ChAdOx1, which is used in the Oxford-AstraZeneca COVID-19 vaccine (also known as Vaxzevria and AZD1222). ChAdOx1 is replication-deficient and derived from a simian adenovirus (ChAd) serotype Y25 engineered by λ red recombination to exchange the native E4 orf4, orf6 and orf6/7 genes for those from human adenovirus HAdV-C5. This vector encodes a codon-optimised full-length spike glycoprotein of SARS-CoV-2 with a tissue plasminogen activator leader sequence (accession no. YP_009724390.1). Other vaccine viral vectors include those used in the Sputnik V or Gam-COVID-Vac vaccine developed by the Gamaleya Research Institute of Epidemiology and Microbiology, the “Ad26.COV2.S” COVID-19 vaccine by Johnson & Johnson and the “AD5-nCOV” COVID-19 vaccine also known as Convidecia developed by CanSino Biologics. Sputnik V or Gam-COVID-Va is a two-vector vaccine i.e., a modified (recombinant) replication-defective adenovirus of a serotype 26 (Ad26) and of serotype 5 (Ad5) each carrying the spike protein-expressing gene of SARS-CoV-2. The viral vaccine vector for the “Ad26.COV2.S” COVID-19 vaccine is a replication-incompetent adenovirus type 26 (Ad26) vector encoding the severe acute respiratory syndrome coronavirus-2 (SARS-CoV-2) spike protein. The “AD5-nCOV” COVID-19 vaccine uses a replication defective adenovirus type-5 (Ad5) viral vector vaccine encoding the spike glycoprotein of SARS-CoV-2.

TABLE 3
summary of vaccines and their corresponding viral vaccine vectors

| Vaccine | Viral vaccine vector |
|---|---|
| ChAdOx1 vaccine developed | A replication-deficient and derived from a simian |
| by Oxford-AstraZeneca | adenovirus (ChAd) serotype Y25 engineered by 2 |
| | red recombination to exchange the native E4 orf4, |
| | orf6 and orf6/7 genes for those from human |
| | adenovirus HAdV-C5. This vector encodes a |
| | codon-optimised full-length spike glycoprotein of |
| | SARS-CoV-2 with a tissue plasminogen activator |
| | leader sequence (accession no. YP_009724390.1). |
| Sputnik V/Gam-COVID-Vac | A two-vector vaccine i.e., a modified (recombinant) |
| vaccine developed by the | replication-defective adenovirus of a serotype 26 |
| Gamaleya Research Institute of | (Ad26) and of serotype 5 (Ad5) each carrying the |
| Epidemiology and | spike protein-expressing gene of SARS-CoV-2. |
| Microbiology | |
| Ad26.COV2.S vaccine by | A replication-incompetent adenovirus type 26 |
| Johnson & Johnson | (Ad26) vector encoding the severe acute respiratory |
| | syndrome coronavirus-2 (SARS-CoV-2) spike |
| | protein |
| AD5-nCOV/Convidecia | A replication defective adenovirus type-5 (Ad5) |
| vaccine developed by CanSino | viral vector vaccine encoding the spike |
| Biologics | glycoprotein of SARS-CoV-2 |

### Bait Molecule—Virion

A bait molecule may be a virion. A virion is the complete virus i.e., a complete virus particular having a DNA or RNA core with a outer protein coat. Thus, a virion may includes spike proteins, envelope proteins, capsid proteins and/or membrane proteins. The virion may have an external envelope such as a fatty membrane. A virion may be live or dead. A virion may be of any virus disclosed herein.

A bait molecule may preferably be a virion that is not capable of replication, such as a pseudovirus.

A pseudovirus may be a synthetic chimera consist of a surrogate viral core, derived from a parent virus, and an envelope glycoprotein on its surface derived from a heterologous virus. The parent virus used to generate a pseudovirus is typically a rhabdovirus (e.g., vesicular stomatitis virus (VSV)) or a retrovirus (e.g., murine leukemia virus (MLV) or human immunodeficiency virus 1 (HIV-1)). The genes inside the pseudovirus are usually altered or modified so that they are unable to produce surface protein on their own. As such, an additional plasmid or stable cell line expressing the surface proteins may be needed to make the pseudovirus. Suitable cell lines may any cell line including human 293T (renal epithelial) cells, human Calu-3 (lung epithelial) cells, and the non-human primate (African Green Monkey) cell line, and Vero-E6 (renal epithelial) cells.

Pseudoviruses are usually engineered to carry reporter genes to make it easier to perform quantitative analyses on these viruses than on wild-type viruses. For example, the parent viral genome is modified to delete essential genes required for replication, including the native envelope glycoprotein gene, and instead, a reporter gene such as a reporter gene coding for luciferase or fluorescent protein is inserted. Pseudoviruses are therefore defective for undergoing a complete replication cycle and may only undergo a single infection cycle. A pseudovirus may or may not have an envelope.

The following may be used as a packaging system to generate a pseudovirus: lentiviral vectors such as those derived from human immunodeficiency virus (HIV-1), simian immunodeficiency virus (SIV), or feline immunodeficiency virus (FIV); a HIV-1 packaging system such as one where HIV genes are selectively cloned into DNA vectors e.g., 2 to 4 plasmids are used as the vectors to minimize viral gene recombination and thereby reduce the possibility of reversion to the WT virus. For example, HIV-1-based systems include those comprising (1) pNL-luc, pNL-GFP, pNL-HSA; pLenti CMV Puro LUC, psPAX2; pDR8.2 (HIV gag-pol), pTrip luciferase; (2) pCMVΔR8.2, pHR′LacZ (β-gal), pHR′EGFP; pR8AEnv, BlaM-Vpr, pcREV; pNL4-3.Luc.R-E-; (3) HIV-1 JR-FL, HIV-1 NL4-3; pNL4-3.Luc.R-E-; (4) pSG3AEnv; pNL4-3.Luc.R-E-; (5) pJK2 (HIV-1-LTR-b-gal); pSG3AEnv; (6) pNL4-3.Luc.R-E-; pCSFLW, p8.91; (7) pNL4-3.Luc.R-E-; pCMVΔR8.2, pHxLacZWP/pHR′-CMVLuc; (8) pCSCGW (pCSC-SP-PW-GFP), pMDL, pRSV-REV; pCMVΔR8.9, FG12/FUhLucW; (9) pSIN-18-PGK-GFP, pCMV R8.74; pCMVΔR8.2, TRIP GFP/HPV-402; (10) pRRLsin-CMV-GFP/pcDNA-HIV-CS-CGW, pMDLg, pRSV/Rev; (11) pCMV-AR8.74, pWPI_F-Luc_BLR/pWPI_Venus-GFP_BLR; (12) pRRLsincppt-CMV-LacZ WPRE, pMD2-LgpRRE, pRSV-Rev; pHR9SIN-cPPT-SGW/pCSFLW, pCMV-A8.91 pSG3AEnv.sCMV.fluc; and (13) pNL4-3.Luc.R-E-; pCMVΔR8.2, pHxLacZWP/pHR′-CMVLuc. A vesicular stomatitis virus packaging (VSV) based system may also be used to generate pseudovirus. A murine leukemia virus based system may also be used to generate pseudovirus. For example, a murine leukemia virus based may comprise (1) pHIT60 (MLV gag-pol); pHIT111; (2) pcGP (MLV gag-pol) pcnbG (MLV LTR); (3) pcGP (MLV gag-pol) pcnbG (MLV LTR); (4) TELCeB6 cells; pTMgp140; FLY-HIV-87-GFP; GP2-2931uc cells; (5) Anjou65-LacZ cells; (6) pCgp (MuLV gag-pol); pMX-GFP; (7) gpnlslacZ cells; pJ6Vpuro; (8) pHIT60 (MLV gag-pol); pMFG-nlsLacZ; MgEGFP-LNGFR; (9) MLV CMV-gag-pol; MLV-GFP; (10) TELCeB6 cells; GP2-293 cell line; pQCXIX (GFP); (11) pCMV-MLV gag-pol; pTG-Luc; (12) GP2-293 cell line; pLNHX; (13) MLV gag/pol; pQCXIX (EGFP); and (14) MLV gag/pol; pkat2βgal; MLV luc constructs. Preferably, a pseudovirus used in the methods described herein is a pseudovirus bearing a protein derived from SARS-CoV-2 such as a S protein or a fragment thereof.

A virion may be an inactivated virus. An inactivated virus may be one which has undergone treatment to reduce or abolish its infectivity. For example, an inactivation may occur by exposing the virus to fat solvents such as ether and/or chloroform. For example, an inactivated virus may be a virus which has been inactivated such that it is safe for administration into an individual and can act as an antigen that provokes an immune response in an individual. A virus can be inactivated by for example, heat inactivation at for example, 56° C. for 30 minutes, 60° C. for 60 minutes or 92° C. for 15 minutes. Different temperatures and times may be required depending on the virus type and viral load. Different viruses may require different treatments to be inactivated.

A virion may be an inert virus. An inert virus may be a virus that does not contain any DNA and/or RNA i.e, it is an unpackaged virus. An inert virus may be a virus that does not contain any DNA or RNA that is necessary for viral replication.

A virion may be a recombinant virion. For example, it may be a recombinant AAV virion comprising a nucleic acid molecule encoding a protein that is missing or defective in an individual.

A virus can be any virus selected from for example, adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2 (SARS-CoV-2), Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus.

A virus may be from the genus, Aalivirus, Aarhusvirus, Abbeymikolonvirus, Abidjanvirus, Abouovirus, Acadevirus, Acadianvirus, Acionnavirus, Actinovirus, Aegirvirus, Aerosvirus, Affertcholeramvirus, Agatevirus, Ageyesisatellite, Aghbyvirus, Agnathovirus, Agricanvirus, Agtrevirus, Ahduovirus, Ahphunavirus, Ahtivirus, Ailurivirus, Albetovirus, Alcyoneusvirus, Alefpapillomavirus, Alexandravirus, Alfamovirus, Allexivirus, Allolevivirus, Almendravirus, Alphaabyssovirus, Alphaarterivirus, Alphabaculovirus, Alphacarmotetravirus, Alphacarmovirus, Alphachrysovirus, Alphacoronavirus, Alphaendornavirus, Alphaentomopoxvirus, Alphafusellovirus, Alphaguttavirus, Alphainfluenzavirus, Alphaletovirus, Alphalipothrixvirus, Alphamesonivirus, Alphamononivirus, Alphanecrovirus, Alphanemrhavirus, Alphanodavirus, Alphanucleorhabdovirus, Alphanudivirus, Alphaovalivirus, Alphapapillomavirus, Alphapartitivirus, Alphapermutotetravirus, Alphapleolipovirus, Alphapolyomavirus, Alphaportoglobovirus, Alpharetrovirus, Alphasphaerolipovirus, Alphaspiravirus, Alphatectivirus, Alphatorquevirus, Alphatrevirus, Alphatristromavirus, Alphaturrivirus, Alphavirus, Amalgavirus, Amdoparvovirus, Amigovirus, Ampelovirus, Ampivirus, Ampullavirus, Ampunavirus, Anamdongvirus, Anaposvirus, Anativirus, Anatolevirus, Andhravirus, Andrewvirus, Andromedavirus, Anphevirus, Antennavirus, Anulavirus, Aokuangvirus, Aparavirus, Apdecimavirus, Aphroditevirus, Aphthovirus, Appavirus, Apricotvirus, Apscaviroid, Aquabirnavirus, Aqualcavirus, Aquamavirus, Aquambidensovirus, Aquaparamyxovirus, Aquareovirus, Arepavirus, Arequatrovirus, Aresaunavirus, Arlivirus, Armstrongvirus, Artiparvovirus, Arurhavirus, Ascovirus, Asfivirus, Ashivirus, Asteriusvirus, Atadenovirus, Atlauavirus, Attisvirus, Attoomivirus, Atuphduovirus, Aumaivirus, Aureusvirus, Aurivirus, Aurunvirus, Austintatiousvirus, Avastrovirus, Avenavirus, Aveparvovirus, Aviadenovirus, Avibirnavirus, Avihepadnavirus, Avihepatovirus, Avipoxvirus, Avisivirus, Avsunviroid, Avunavirus, Axomammavirus, Ayakvirus, Ayaqvirus, Babusatellite, Babuvirus, Bacillarnavirus, Badaguanvirus, Badnavirus, Bafinivirus, Baikalvirus, Baltimorevirus, Banchanvirus, Bandavirus, Bantamvirus, Baoshanvirus, Barbavirus, Barhavirus, Barnavirus, Barnyardvirus, Bastillevirus, Batrachovirus, Bavovirus, Baxtervirus, Baylorvirus, Bcepmuvirus, Bdellomicrovirus, Becurtovirus, Beetrevirus, Begomovirus, Beidivirus, Bellamyvirus, Bendigovirus, Benyvirus, Bequatrovirus, Berhavirus, Berlinvirus, Bernalvirus, Bertelyvirus, Betaarterivirus, Betabaculovirus, Betacarmovirus, Betachrysovirus, Betacoronavirus, Betaendornavirus, Betaentomopoxvirus, Betafusellovirus, Betaguttavirus, Betainfluenzavirus, Betalipothrixvirus, Betanecrovirus, Betanodavirus, Betanucleorhabdovirus, Betanudivirus, Betapapillomavirus, Betapartitivirus, Betapleolipovirus, Betapolyomavirus, Betaretrovirus, Betasatellite, Betasphaerolipovirus, Betatectivirus, Betatetravirus, Betatorquevirus, Betterkatzvirus, Bevemovirus, Bicaudavirus, Bidensovirus, Bielevirus, Bifilivirus, Bifseptvirus, Bignuzvirus, Bingvirus, Biquartavirus, Biseptimavirus, Bixzunavirus, Bjornvirus, Blattambidensovirus, Blosnavirus, Blunervirus, Bocaparvovirus, Bolenivirus, Bongovirus, Bonnellvirus, Boosepivirus, Bopivirus, Bostovirus, Botoulivirus, Botrexvirus, Botybirnavirus, Bovismacovirus, Bovispumavirus, Bowservirus, Bracovirus, Brambyvirus, Brevihamaparvovirus, Bridgettevirus, Brigitvirus, Britbratvirus, Brizovirus, Bromovirus, Bronvirus, Brujitavirus, Brunovirus, Brussowvirus, Bruynoghevirus, Bucovirus, Busanvirus, Buttersvirus, Bymovirus, Caeruleovirus, Cafeteriavirus, Caligrhavirus, Camvirus, Canoevirus, Capillovirus, Capistrivirus, Capripoxvirus, Capulavirus, Carbovirus, Cardiovirus, Cardoreovirus, Carlavirus, Carltongylesvirus, Caroctavirus, Carpasinavirus, Casadabanvirus, Catalunyavirus, Caulimovirus, Cavemovirus, Cbastvirus, Cecivirus, Ceduovirus, Ceetrepovirus, Celavirus, Centapoxvirus, Cepunavirus, Cequinquevirus, Certrevirus, Cervidpoxvirus, Cetovirus, Chakrabartyvirus, Chaphamaparvovirus, Charlievirus, Charybdisvirus, Charybnivirus, Chatterjeevirus, Chenonavirus, Cheoctovirus, Cheravirus, Cheungvirus, Chiangmaivirus, Chimshavirus, Chipapillomavirus, Chipolycivirus, Chivirus, Chlamydiamicrovirus, Chloriridovirus, Chlorovirus, Chordovirus, Chosvirus, Christensenvirus, Chunghsingvirus, Cilevirus, Cimpunavirus, Cinunavirus, Circovirus, Citexvirus, Citrivirus, Clavavirus, Clecrusatellite, Closterovirus, Clostunsatellite, Cocadviroid, Coccolithovirus, Coetzeevirus, Coguvirus, Colecusatellite, Coleviroid Colossusvirus, Coltivirus, Comovirus, Coopervirus, Copiparvovirus, Coralvirus, Coriovirus, Corndogvirus, Cornellvirus, Corticovirus, Cosavirus, Cosmacovirus, Crahelivirus, Crinivirus, Cripavirus, Crocodylidpoxvirus, Crohivirus, Cronosvirus, Cronusvirus, Crustavirus, Cryspovirus, Cucumovirus, Cuernavacavirus, Cuevavirus, Cultervirus, Curiovirus, Curtovirus, Cyclitvirus, Cyclophivirus, Cyclovirus, Cymopoleiavirus, Cynoglossusvirus, Cypovirus, Cyprinivirus, Cystovirus, Cytomegalovirus, Cytorhabdovirus, Daemvirus, Daredevilvirus, Decapodiridovirus, Decurrovirus, Delepquintavirus, Deltaarterivirus, Deltabaculovirus, Deltacoronavirus, Deltaentomopoxvirus, Deltaflexivirus, Deltainfluenzavirus, Deltalipothrixvirus, Deltapapillomavirus, Deltapartitivirus, Deltapolyomavirus, Deltaretrovirus, Deltasatellite, Deltatorquevirus, Deltavirus, Demosthenesvirus, Dependoparvovirus, Derbicusvirus, Detrevirus, Dhakavirus, Dhillonvirus, Dianlovirus, Dianthovirus, Diatodnavirus, Dichorhavirus, Dicipivirus, Diegovirus, Dinodnavirus, Dinornavirus, Dinovernavirus, Dioscovirus, Diresapivirus, Dismasvirus, Divavirus, Doucettevirus, Douglaswolinvirus, Dragsmacovirus, Dronavirus, Drosmacovirus, Drulisvirus, Dyochipapillomavirus, Dyodeltapapillomavirus, Dyoepsilonpapillomavirus, Dyoetapapillomavirus, Dyoiotapapillomavirus, Dyokappapapillomavirus, Dyolambdapapillomavirus, Dyomupapillomavirus, Dyonupapillomavirus, Dyoomegapapillomavirus, Dyoomikronpapillomavirus, Dyophipapillomavirus, Dyopipapillomavirus, Dyopsipapillomavirus, Dyorhopapillomavirus, Dyosigmapapillomavirus, Dyotaupapillomavirus, Dyothetapapillomavirus, Dyoupsilonpapillomavirus, Dyoxipapillomavirus, Dyozetapapillomavirus, Eapunavirus, Eastlansingvirus, Ebolavirus, Eclunavirus, Edenvirus, Efquatrovirus, Eganvirus, Eiauvirus, Eisenstarkvirus, Elaviroid Elerivirus, Elunavirus, Elvirus, Emalynvirus, Emaravirus, Emdodecavirus, Enamovirus, Eneladusvirus, Enhodamvirus, Enquatrovirus, Enterovirus, Entnonagintavirus, Entomobirnavirus, Entovirus, Ephemerovirus, Eponavirus, Epseptimavirus, Epsilonarterivirus, Epsilonpapillomavirus, Epsilonretrovirus, Epsilontorquevirus, Equispumavirus, Eracentumvirus, Eragrovirus, Erbovirus, Ermolevavirus, Errantivirus, Erskinevirus, Erythroparvovirus, Etaarterivirus, Etapapillomavirus, Etatorquevirus, Eurybiavirus, Eyrevirus, Fabavirus, Fabenesatellite, Fairfaxidumvirus, Farahnazvirus, Faunusvirus, Felipivirus, Felispumavirus, Felixounavirus, Felsduovirus, Feofaniavirus, Feravirus, Ferlavirus, Fibralongavirus, Fibrovirus, Ficleduovirus, Fijivirus, Finnlakevirus, Fipivirus, Fipvunavirus, Firehammervirus, Fischettivirus, Fishburnevirus, Flaumdravirus, Flavivirus, Fletchervirus, Foetvirus, Foturvirus, Foussvirus, Foveavirus, Franklinbayvirus, Friunavirus, Fromanvirus, Furovirus, Fussvirus, Gaiavirus, Gajwadongvirus, Galaxyvirus, Gallantivirus, Gallivirus, Galunavirus, Gamaleyavirus, Gammaarterivirus, Gammabaculovirus, Gammacarmovirus, Gammacoronavirus, Gammaentomopoxvirus, Gammainfluenzavirus, Gammalipothrixvirus, Gammanucleorhabdovirus, Gammapapillomavirus, Gammapartitivirus, Gammapleolipovirus, Gammapolyomavirus, Gammaretrovirus, Gammasphaerolipovirus, Gammatectivirus, Gammatorquevirus, Gamtrevirus, Gaprivervirus, Gelderlandvirus, Gemycircularvirus, Gemyduguivirus, Gemygorvirus, Gemykibivirus, Gemykolovirus, Gemykrogvirus, Gemykroznavirus, Gemytondvirus, Gemyvongvirus, Gequatrovirus, Gesputvirus, Getalongvirus, Getseptimavirus, Ghobesvirus, Ghunavirus, Giardiavirus, Giessenvirus, Gilesvirus, Gillianvirus, Globulovirus, Glossinavirus, Godonkavirus, Gofduovirus, Goodmanvirus, Goravirus, Gordonvirus, Gordtnkvirus, Gorganvirus, Gorjumvirus, Goslarvirus, Gosmusatellite, Goukovirus, Grablovirus, Gruhelivirus, Grusopivirus, Guelphvirus, Gustavvirus, Gutovirus, Gyeonggidovirus, Gyeongsanvirus, Gyrovirus, Haartmanvirus, Habenivirus, Hanrivervirus, Hapavirus, Hapunavirus, Harbinvirus, Harkavirus, Harrisonvirus, Hartmanivirus, Hawkeyevirus, Hedwigvirus, Heilongjiangvirus, Helsettvirus, Helsingorvirus, Hemiambidensovirus, Hemipivirus, Hemivirus, Hendrixvirus, Henipavirus, Hepacivirus, Hepanhamaparvovirus, Hepatovirus, Herbevirus, Herpetohepadnavirus, Hexartovirus, Higashivirus, Higrevirus, Hiyaavirus, Hollowayvirus, Holosalinivirus, Homburgvirus, Hongcheonvirus, Hoplichthysvirus, Hordeivirus, Horusvirus, Horwuvirus, Hostuviroid, Hpunavirus, Hubavirus, Hubramonavirus, Huchismacovirus, Hudivirus, Hudovirus, Hunnivirus, Hupolycivirus, Hypovirus, Iapetusvirus, Ichnovirus, Ichtadenovirus, Ichthamaparvovirus, Ictalurivirus, Idaeovirus, Idnoreovirus, Iflavirus, Igirivirus, Ikedavirus, Ilarvirus, Iltovirus, llzatvirus, Incheonvrus, Infratovirus, Infulavirus, Inhavirus, Inovirus, Invictavirus, Iodovirus, Ionavirus, Iotaarterivirus, Iotapapillomavirus, Iotatorquevirus, Ipomovirus, Iridovirus, Irtavirus, Isavirus, Iteradensovirus, Ithacavirus, Ixovirus, Jacevirus, Jalkavirus, Jarilovirus, Jasminevirus, Jedunavirus, Jeilongvirus, Jenstvirus, Jerseyvirus, Jesfedecavirus, Jiaodavirus, Jiaoyazivirus, Jilinvirus, Jimmervirus, Johnsonvirus, Jonvirus, Juiceboxvirus, Jwalphavirus, Kafavirus, Kafunavirus, Kagunavirus, Kairosalinivirus, Kajamvirus, Kakivirus, Kalppathivirus, Kanaloavirus, Kantovirus, Kaohsiungvirus, Kappaarterivirus, Kappapapillomavirus, Kappatorquevirus, Karamvirus, Kayfunavirus, Kayvirus, Kelleziovirus, Kelmasvirus, Kelquatrovirus, Kembevirus, Kieseladnavirus, Kilunavirus, Kirikabuvirus, Kisquattuordecimvirus, Kisquinquevirus, Kleczkowskavirus, Klementvirus, Kobuvirus, Kochikohdavirus, Kochitakasuvirus, Kojivirus, Kolesnikvirus, Korravirus, Kostyavirus, Kotilavirus, Koutsourovirus, Krakvirus, Krampusvirus, Krischvirus, Krylovvirus, Kryptosalinivirus, Kukrinivirus, Kunsagivirus, Kuravirus, Kusarnavirus, Kuttervirus, Labyrnavirus, Lacusarxvirus, Lagaffevirus, Lagovirus, Lambdaarterivirus, Lambdapapillomavirus, Lambdatorquevirus, Lambdavirus, Lanavirus, Laroyevirus, Laulavirus, Lauvirus, Ledantevirus, Lederbergvirus, Leishmaniavirus, Lentavirus, Lentinuvirus, Lentivirus, Lenusvirus, Leporipoxvirus, Lessievirus, Leucotheavirus, Levivirus, Libanvirus, Lidleunavirus, Liebevirus, Liefievirus, Lightbulbvirus, Likavirus, Lilyvirus, Limdunavirus, Limelightvirus, Limestonevirus, Limnipivirus, Lincruvirus, Lindendrivevirus, Lineavirus, Lingvirus, Lirvirus, Litunavirus, Livupivirus, Llyrvirus, Loanvirus, Locarnavirus, Loessnervirus, Lokivirus, Lolavirus, Lomovskayavirus, Longwoodvirus, Loriparvovirus, Lostrhavirus, Loudonvirus, Loughboroughvirus, Lubbockvirus, Luckybarnesvirus, Luckytenvirus, Ludopivirus, Lughvirus, Lullwatervirus, Luteovirus, Luzseptimavirus, Lwoffvirus, Lyctovirus, Lymphocryptovirus, Lymphocystivirus, Lyssavirus, Macanavirus, Macavirus, Machinavirus, Machlomovirus, Macluravirus, Macronovirus, Macropopoxvirus, Maculavirus, Maculvirus, Maenadvirus, Magadivirus, Magoulivirus, Majavirus, Malagasivirus, Mamastrovirus, Mammarenavirus, Mandarivirus, Manhattanvirus, Mapvirus, Marafivirus, Marburgvirus, Mardecavirus, Mardivirus, Marnavirus, Marseillevirus, Marthavirus, Marvinvirus, Mastadenovirus, Mastrevirus, Mavirus, Maxrubnervirus, Mazuvirus, Megabirnavirus, Megalocytivirus, Megrivirus, Mementomorivirus, Metaavulavirus, Metahepadnavirus, Metamorphoovirus, Metapneumovirus, Metavirus, Metrivirus, Mguuvirus, Mieseafarmvirus, Milvetsatellite, Mimasvirus, Mimivirus, Mimoreovirus, Miniambidensovirus, Minipunavirus, Minovirus, Minunavirus, Mischivirus, Mitovirus, Mivedwarsatellite, Mivirus, Mobatvirus, Mobuvirus, Moineauvirus, Molluscipoxvirus, Montyvirus, Mooglevirus, Moonvirus, Morbillivirus, Mosavirus, Mosigvirus, Mousrhavirus, Muarterivirus, Mudcatvirus, Mukerjeevirus, Mupapillomavirus, Mupivirus, Murciavirus, Muromegalovirus, Murrayvirus, Muscavirus, Mushuvirus, Mustelpoxvirus, Mutorquevirus, Muvirus, Mycoflexivirus, Mycoreovirus, Myohalovirus, Myrropivirus, Myunavirus, Myxoctovirus, Nacovirus, Naesvirus, Namakavirus, Nampongvirus, Nanhaivirus, Nankokuvirus, Nanovirus, Napahaivirus, Narmovirus, Narnavirus, Nazgulvirus, Nebovirus, Neferthenavirus, Negarvirus, Nepovirus, Neptunevirus, Nereusvirus, Nerrivikvirus, Nickievirus, Ningirsuvirus, Nipunavirus, Nitmarvirus, Nitunavirus, Nodensvirus, Nohivirus, Nonagvirus, Nonanavirus, Norovirus, Nouzillyvirus, Novirhabdovirus, Novosibovirus, Novosibvirus, Noxifervirus, Nuarterivirus, Nupapillomavirus, Nutorquevirus, Nyavirus, Nyceiraevirus, Nyfulvavirus, Nymphadoravirus, Obolenskvirus, Oengusvirus, Ohlsrhavirus, Oinezvirus, Okavirus, Okubovirus, Oleavirus, Omegapapillomavirus, Omegatetravirus, Omegavirus, Omikronpapillomavirus, Oncotshavirus, Oneupvirus, Ophiovirus, Orbivirus, Orchidvirus, Orinovirus, Orivirus, Orthoavulavirus, Orthobornavirus, Orthobunyavirus, Orthohantavirus, Orthohepadnavirus, Orthohepevirus, Orthonairovirus, Orthophasmavirus, Orthopneumovirus, Orthopoxvirus, Orthoreovirus, Orthorubulavirus, Orthotospovirus, Oryzavirus, Oryzopoxvirus, Oscivirus, Oshimavirus, Oslovirus, Ostreavirus, Otagovirus, Ourmiavirus, Paadamvirus, Pacuvirus, Pagavirus, Pagevirus, Paguronivirus, Pahexavirus, Pahsextavirus, Pairvirus, Pakpunavirus, Palaemonvirus, Pamexvirus, Panicovirus, Panjvirus, Papanivirus, Papyrusvirus, Paraavulavirus, Parabovirus, Parahepadnavirus, Parapoxvirus, Pararubulavirus, Parechovirus, Parhipatevirus, Pasivirus, Passerivirus, Patiencevirus, Pbilvirus, Pbunavirus, Peatvirus, Pecentumvirus, Pecluvirus, Pedosvirus, Peduovirus, Pefuambidensovirus, Pegivirus, Pegunavirus, Pekhitvirus, Pektosvirus, Pelagivirus, Pelamoviroid, Pelarspovirus, Pemapivirus, Penstylhamaparvovirus, Pepyhexavirus, Percavirus, Percyvirus, Perhabdovirus, Perisivirus, Peropuvirus, Pestivirus, Petsuvirus, Pettyvirus, Petuvirus, Phabquatrovirus, Phaeovirus, Phapecoctavirus, Phasivirus, Phayoncevirus, Phietavirus, Phifelvirus, Phikmvvirus, Phikzvirus, Phimunavirus, Phipapillomavirus, Phistoryvirus, Phitrevirus, Phlebovirus, Phutvirus, Phytoreovirus, Picardvirus, Picobirnavirus, Pidchovirus, Piedvirus, Pienvirus, Pifdecavirus, Pijolavirus, Pikminvirus, Pipapillomavirus, Pipefishvirus, Piscihepevirus, Plaisancevirus, Plasmavirus, Platypuvirus, Playavirus, Plectrovirus, Plotvirus, Poacevirus, Podivirus, Poecivirus, Pogseptimavirus, Poindextervirus, Pokrovskaiavirus, Polemovirus, Polerovirus, Pollyceevirus, Polybotosvirus, Polymycovirus, Pomovirus, Pontunivirus, Pontusvirus, Popoffvirus, Porprismacovirus, Poseidonvirus, Pospiviroid, Potamipivirus, Potexvirus, Potyvirus, Poushouvirus, Powvirus, Pradovirus, Prasinovirus, Pregotovirus, Primolicivirus, Priunavirus, Proboscivirus, Prosimiispumavirus, Protoambidensovirus, Protobacilladnavirus, Protoparvovirus, Prunevirus, Prymnesiovirus, Przondovirus, Psavirus, Psecadovirus, Pseudovirus, Psimunavirus, Psipapillomavirus, Pteridovirus, Pteropopoxvirus, Pulverervirus, Punavirus, Qadamvirus, Qingdaovirus, Quadrivirus, Quaranjavirus, Quhwahvirus, Rabovirus, Radnorvirus, Rafivirus, Raleighvirus, Ranavirus, Raphidovirus, Rauchvirus, Ravavirus, Ravinvirus, Recovirus, Redivirus, Reginaelenavirus, Reptarenavirus, Reptillovirus, Rerduovirus, Respirovirus, Restivirus, Reyvirus, Rhadinovirus, Rhizidiovirus, Rhopapillomavirus, Rigallievirus, Rimavirus, Ripduovirus, Risingsunvirus, Risjevirus, Robigovirus, Rogerhendrixvirus, Rogunavirus, Rohelivirus, Ronaldovirus, Ronavirus, Rosadnavirus, Rosavirus, Rosebushvirus, Rosemountvirus, Rosenblumvirus, Roseolovirus, Rotavirus, Roufvirus, Rowavirus, Roymovirus, Rtpvirus, Rubivirus, Rubodvirus, Rudivirus, Ruthyvirus, Rymovirus, Ryyoungvirus, Saclayvirus, Sadwavirus, Saetivirus, Sajorinivirus, Sakobuvirus, Salacisavirus, Salasvirus, Salemvirus, Salisharnavirus, Salivirus, Salmondvirus, Salmonivirus, Salmonpoxvirus, Salovirus, Salterprovirus, Samistivirus, Samunavirus, Samwavirus, Sanovirus, Sansavirus, Sapelovirus, Saphexavirus, Sapovirus, Sashavirus, Sasquatchvirus, Sasvirus, Sauletekiovirus, Saundersvirus, Sawastrivirus, Sawgrhavirus, Scapunavirus, Schiekvirus, Schizotequatrovirus, Schmidvirus, Schmittlotzvirus, Schnabeltiervirus, Schubertvirus, Scindoambidensovirus, Sciuripoxvirus, Sciuriunavirus, Sclerodarnavirus, Sclerotimonavirus, Scleroulivirus, Scoliodonvirus, Scottvirus, Scutavirus, Scuticavirus, Seadornavirus, Sectovirus, Sednavirus, Semotivirus, Senecavirus, Senquatrovirus, Seongnamvirus, Seoulvirus, Septimatrevirus, Sepunavirus, Sequivirus, Serkorvirus, Sertoctavirus, Seunavirus, Seuratvirus, Seussvirus, Sextaecvirus, Shalavirus, Shanbavirus, Shangavirus, Shapirovirus, Shaspivirus, Shenzhenvirus, Shilevirus, Shizishanvirus, Siadenovirus, Sicinivirus, Sieqvirus, Sigmapapillomavirus, Sigmavirus, Silviavirus, Simiispumavirus, Siminovitchvirus, Simpcentumvirus, Simplexvirus, Sinaivirus, Sinsheimervirus, Sirevirus, Sitaravirus, Skarprettervirus, Skunavirus, Slashvirus, Slopekvirus, Smoothievirus, Sobemovirus, Socyvirus, Sogarnavirus, Solendovirus, Sonalivirus, Sophoyesatellite, Sopolycivirus, Sortsnevirus, Soupsvirus, Sourvirus, Soymovirus, Spbetavirus, Spiromicrovirus, Sprivivirus, Sputnikvirus, Squashvirus, Sripuvirus, Staminivirus, Stanholtvirus, Steinhofvirus, Stockinghallvirus, Stompelvirus, Stompvirus, Stopalavirus, Stopavirus, Striavirus, Striwavirus, Stubburvirus, Stupnyavirus, Subclovsatellite, Subteminivirus, Sugarlandvirus, Suipoxvirus, Sukuvirus, Sunrhavirus, Sunshinevirus, Suspvirus, Suturavirus, Suwonvirus, Svunavirus, Symapivirus, Synodonvirus, Tabernariusvirus, Taipeivirus, Tamkungvirus, Tangaroavirus, Tankvirus, Tapwovirus, Taranisvirus, Taupapillomavirus, Tawavirus, Teetrevirus, Tefnutvirus, Tegunavirus, Telnavirus, Tenuivirus, Tepovirus, Tequatrovirus, Tequintavirus, Terapinvirus, Tertilicivirus, Teschovirus, Teseptimavirus, Tetraparvovirus, Thamnovirus, Thaumasvirus, Thetaarterivirus, Thetapapillomavirus, Thetatorquevirus, Thetisvirus, Thogotovirus, Thomixvirus, Thornevirus, Thottimvirus, Tiamatvirus, Tibrovirus, Tidunavirus, Tigrvirus, Tigunavirus, Tiilvirus, Tijeunavirus, Tilapinevirus, Timquatrovirus, Tinduovirus, Titanvirus, Tlsvirus, Tobamovirus, Tobravirus, Tombusvirus, Topocuvirus, Torbevirus, Torchivirus, Torovirus, Torradovirus, Tortellinivirus, Totivirus, Tottorivirus, Toursvirus, Toutatisvirus, Traversvirus, Treisdeltapapillomavirus, Treisepsilonpapillomavirus, Treisetapapillomavirus, Treisiotapapillomavirus, Treiskappapapillomavirus, Treisthetapapillomavirus, Treiszetapapillomavirus, Tremovirus, Triatovirus, Triavirus, Trichomonasvirus, Trichovirus, Trigintaduovirus, Trinavirus, Trinevirus, Trippvirus, Tritimovirus, Tritonvirus, Troedvirus, Tropivirus, Trungvirus, Tsarbombavirus, Tulanevirus, Tunavirus, Tunggulviirus, Tungrovirus, Tuodvirus, Tupavirus, Turncurtovirus, Turrinivirus, Twortvirus, Tymovirus, Uetakevirus, Uliginvirus, Umbravirus, Unahavirus, Unaquatrovirus, Unyawovirus, Upsilonpapillomavirus, Uukuvirus, Vaccinivirus, Valovirus, Varicellovirus, Varicosavirus, Vashvirus, Vectrevirus, Vegasvirus, Velarivirus, Vellamovirus, Vendettavirus, Vequintavirus, Versovirus, Vesiculovirus, Vesivirus, Vespertilionpoxvirus, Vespertiliovirus, Vhmlvirus, Vhulanivirus, Vicialiavirus, Vicosavirus, Victorivirus, Vidquintavirus, Vieuvirus, Villovirus, Vilniusvirus, Vipunavirus, Virtovirus, Vitivirus, Viunavirus, Vividuovirus, Voetvirus, Vojvodinavirus, Votkovvirus, Vulnificusvirus, W, Waewaevirus, Waikavirus, Wamavirus, Wanjuvirus, Warwickvirus, Wbetavirus, Weaselvirus, Webervirus, Wellingtonvirus, Wenrivirus, Whispovirus, Wifcevirus, Wildcatvirus, Wilnyevirus, Wilsonroadvirus, Winklervirus, Wizardvirus, Woesvirus, Woodruffvirus, Wphvirus, Wuhanvirus, Wuhivirus, Wumivirus, X, Xiamenvirus, Xipapillomavirus, Xipdecavirus, Xuanwuvirus, Xuquatrovirus, Xylivirus, Y, Yangvirus, Yatapoxvirus, Yingvirus, Yokohamavirus, Yoloswagvirus, Yuavirus, Yushanvirus, Yuyuevirus, Yvonnevirus, Zarhavirus, Zeavirus, Zetaarterivirus, Zetapapillomavirus, Zetatorquevirus, Zetavirus, Zindervirus, Zybavirus, Subgenera, Ampobartevirus, Andecovirus, Aplyccavirus, Balbicanovirus, Behecravirus, Beturrivirus, Blicbavirus, Bosnitovirus, Brangacovirus, Buldecovirus, Casualivirus, Cegacovirus, Chibartevirus, Cholivirus, Colacovirus, Cradenivirus, Debiartevirus, Decacovirus, Dumedivirus, Duvinacovirus, Embecovirus, Enselivirus, Eurpobartevirus, Hanalivirus, Hedartevirus, Hepoptovirus, Herdecovirus, Hibecovirus, Igacovirus, Kadilivirus, Kaftartevirus, Karsalivirus, Kigiartevirus, Luchacovirus, Menolivirus, Merbecovirus, Micartevirus, Milecovirus, Minacovirus, Minunacovirus, Mitartevirus, Myotacovirus, Namcalivirus, Nobecovirus, Nyctacovirus, Ofalivirus, Pedacovirus, Pedartevirus, Pimfabavirus, Rebatovirus, Renitovirus, Rhinacovirus, Roypretovirus, Salnivirus, Sanematovirus, Sarbecovirus, Satsumavirus, Setracovirus, Sheartevirus, Snaturtovirus, Soracovirus, Stramovirus, Sunacovirus, Tegacovirus, Tilitovirus, Tipravirus, Wenilivirus, Xintolivirus.

A virus may be from the family, Abyssoviridae, Ackermannviridae, Adenoviridae, Adintoviridae, Aliusviridae, Alloherpesviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Atkinsviridae, Autographiviridae, Avsunviroidae, Bacilladnaviridae, Baculoviridae, Barnaviridae, Belpaoviridae, Benyviridae, Betaflexiviridae, Bicaudaviridae, Bidnaviridae, Birnaviridae, Blumeviridae, Bornaviridae, Botourmiaviridae, Bromoviridae, Caliciviridae, Carmotetraviridae, Caulimoviridae, Chaseviridae, Chrysoviridae, Chuviridae, Circoviridae, Clavaviridae, Closteroviridae, Coronaviridae, Corticoviridae, Cremegaviridae, Crepuscuviridae, Cruliviridae, Curvulaviridae, Cystoviridae, Deltaflexiviridae, Demerecviridae, Dicistroviridae, Drexlerviridae, Duinviridae, Endornaviridae, Euroniviridae, Fiersviridae, Filoviridae, Fimoviridae, Finnlakeviridae, Flaviviridae, Fuselloviridae, Gammaflexiviridae, Geminiviridae, Genomoviridae, Globuloviridae, Gresnaviridae, Guelinviridae, Guttaviridae, Halspiviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Kolmioviridae, Lavidaviridae, Leishbuviridae, Lipothrixviridae, Lispiviridae, Malacoherpesviridae, Marnaviridae, Marseilleviridae, Matonaviridae, Matshushitaviridae, Mayoviridae, Medioniviridae, Megabirnaviridae, Mesoniviridae, Metaviridae, Metaxyviridae, Microviridae, Mimiviridae, Mitoviridae, Mononiviridae, Mymonaviridae, Myoviridae, Mypoviridae, Myriaviridae, Nairoviridae, Nanghoshaviridae, Nanhypoviridae, Nanoviridae, Narnaviridae, Natareviridae, Nimaviridae, Nodaviridae, Nudiviridae, Nyamiviridae, Olifoviridae, Orthomyxoviridae, Ovaliviridae, Papillomaviridae, Paramyxoviridae, Partitiviridae, Parvoviridae, Paulinoviridae, Peribunyaviridae, Permutotetraviridae, Phasmaviridae, Phenuiviridae, Phycodnaviridae, Picobirnaviridae, Picornaviridae, Plasmaviridae, Plectroviridae, Pleolipoviridae, Pneumoviridae, Podoviridae, Polycipiviridae, Polydnaviridae, Polymycoviridae, Polyomaviridae, Portogloboviridae, Pospiviroidae, Potyviridae, Poxviridae, Pseudoviridae, Qinviridae, Quadriviridae, Redondoviridae, Reoviridae, Retroviridae, Rhabdoviridae, Roniviridae, Rountreeviridae, Rudiviridae, Salasmaviridae, Sarthroviridae, Schitoviridae, Secoviridae, Simuloviridae, Sinhaliviridae, Siphoviridae, Smacoviridae, Solemoviridae, Solinviviridae, Solspiviridae, Sphaerolipoviridae, Spiraviridae, Steitzviridae, Sunviridae, Tectiviridae, Thaspiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, Yueviridae, Zobellviridae.

A virus may be from the subfamily Actantavirinae, Agantavirinae, Aglimvirinae, Alphaherpesvirinae, Alphairidovirinae, Alpharhabdovirinae, Arquatrovirinae, Avulavirinae, Azeredovirinae, Bastillevirinae, Bclasvirinae, Beephvirinae, Beijerinckvirinae, Betaherpesvirinae, Betairidovirinae, Betarhabdovirinae, Braunvirinae, Brockvirinae, Bronfenbrennervirinae, Bullavirinae, Calvusvirinae, Ceronivirinae, Chebruvirinae, Chimanivirinae, Chordopoxvirinae, Cleopatravirinae, Cobavirinae, Colwellvirinae, Comovirinae, Corkvirinae, Crocarterivirinae, Crustonivirinae, Cvivirinae, Dclasvirinae, Deejayvirinae, Denniswatsonvirinae, Densovirinae, Dolichocephalovirinae, Eekayvirinae, Emmerichvirinae, Enquatrovirinae, Entomopoxvirinae, Equarterivirinae, Ermolyevavirinae, Erskinevirinae, Eucampyvirinae, Firstpapillomavirinae, Fuhrmanvirinae, Gammaherpesvirinae, Gammarhabdovirinae, Geminialphasatellitinae, Gochnauervirinae, Gofosavirinae, Gokushovirinae, Gorgonvirinae, Guernseyvirinae, Gutmannvirinae, Hamaparvovirinae, Hendrixvirinae, Heroarterivirinae, Hexponivirinae, Humphriesvirinae, Hyporhamsavirinae, Jasinkavirinae, Krylovirinae, Langleyhallvirinae, Letovirinae, Mammantavirinae, Markadamsvirinae, Mccleskeyvirinae, Mccorquodalevirinae, Mclasvirinae, Medionivirinae, Melnykvirinae, Metaparamyxovirinae, Migulavirinae, Molineuxvirinae, Mononivirinae, Nanoalphasatellitinae, Nclasvirinae, Nefertitivirinae, Northropvirinae, Nymbaxtervirinae, Okabevirinae, Okanivirinae, Orthocoronavirinae, Orthoparamyxovirinae, Orthoretrovirinae, Ounavirinae, Parvovirinae, Pclasvirinae, Peduovirinae, Petromoalphasatellitinae, Picovirinae, Piscanivirinae, Pontosvirinae, Procedovirinae, Queuovirinae, Quinvirinae, Rakietenvirinae, Regressovirinae, Remotovirinae, Repantavirinae, Reternivirinae, Rhodovirinae, Rodepovirinae, Rogunavirinae, Rothmandenesvirinae, Rubulavirinae, Sarlesvirinae, Secondpapillomavirinae, Sedoreovirinae, Sepvirinae, Serpentovirinae, Simarterivirinae, Skryabinvirinae, Slopekvirinae, Spinareovirinae, Spounavirinae, Spumaretrovirinae, Studiervirinae, Tatarstanvirinae, Tempevirinae, Tevenvirinae, Tiamatvirinae, Torovirinae, Trabyvirinae, Trivirinae, Tunavirinae, Tunicanivirinae, Twarogvirinae, Twortvirinae, Tybeckvirinae, Variarterivirinae, Vequintavirinae, Zealarterivirinae.

Preferably a virion is a coronavirus virion. More preferably, the virion is a severe acute respiratory syndrome coronavirus 2 (SARS-CoV-2) virion, such as a wild-type SARS-CoV-2 virion. The virion may be a variant of SARS-CoV-2 as disclosed herein.

A plurality of bait molecules may be used in the invention disclosed herein. A “bait molecule” and “a plurality of the bait molecules” is understood to mean that the bait molecules in a single reaction are identical, i.e. they all of the same species. For example, the plurality of bait molecules may be a population of identical spike proteins or a population of identical virions rather than a mixed population of different proteins or virions.

A plurality of identical bait molecules may be produced by any means known in the art. A plurality of identical bait molecules may be produced by recombinant means, such that all bait molecules are identical or substantially identical. Accordingly, a plurality of identical bait molecules may be a plurality of recombinant bait molecules.

A bait molecule may comprise one or more mutations and/or modifications as defined further herein.

### Polypeptide

As described herein, a polypeptide may comprise the amino acid sequence of the whole or a portion of a polypeptide. A polypeptide disclosed herein may be a full-length sequence of a polypeptide, for example such as a full-length sequence of a SARS-CoV2 spike protein. A polypeptide disclosed herein may be a portion of a polypeptide such as a SARS-CoV2 spike protein.

### Fragment

A “fragment” as described herein, is any polypeptide which has an amino acid sequence which is shorter than the relevant full-length polypeptide. The skilled person may readily delineate the sequence characteristics which define a full-length polypeptide, such as by sequence alignment and analysis. Accordingly, a skilled person may readily delineate such a “fragment”.

### Variant

The amino acid sequence of a variant differs from the amino acid sequence of a reference, such as a wild-type, by one or more amino acid mutations. For example, a variant may be a SARS-CoV2 variant of concern as described herein. A variant may be a SARS-CoV2 variant of interest as described herein.

### Mutations

The term mutation encompasses amino acid additions, deletions or substitutions made relative to a reference, such as a wild-type protein or fragment thereof. The term mutation encompasses nucleic acid additions, deletions or substitutions made relative to a reference, such as a wild-type nucleic acid sequence or fragment thereof. The term mutation may be used to distinguish a variant from a reference, such as a wild-type protein or wild-type virus. For example, a mutation at position E484K of SARS-CoV-2 refers to substitution of glutamic acid (E) at position 484 with lysine (K) of the original “Wuhan” SARS-CoV-2 protein.

### Modifications

Modifications may include those provided by an enzyme such the attachment of: hydrophobic groups for membrane localization, cofactors for enhanced enzymatic activity, modifications of translation factors, chemical groups. Modifications may also include attachments that do not require an enzyme. Modifications may include the attachment of proteins, attachment of chemical groups, chemical modification of amino acids, and/or other structural changes. Modifications thus may include acylation, myristoylation, palmitoylation, isoprenylation, prenylation, farnesylation, geranylgeranylation, glypiation, lipoylation, attachment of a flavin moiety, attachment of heme C, phosphopantetheinylation, retinylidene Schiff base formation, diphthamide, attachment of ethanolamine phosphoglycerol, hypusine formation, beta-Lysine attachment, acylation (such as O-acylation N-acylation and/or S-acylation), acetylation, deacetylation, formylation, alkylation, methylation, demethylation, amidation, arginylation, polyglutamylation, polyglycylation, butyrylation, gamma-carboxylation, glycosylation, O-GlcNAc addition, polysialylation, malonylation, hydroxylation, iodination, phosphate ester (O-linked) or phosphoramidate (N-linked) formation, phosphorylation (O-linked and/or N-linked), adenylylation (O-linked or N-linked), uridylylation, propionylation, pyroglutamate formation, S-glutathionylation, S-nitrosylation, S-sulfenylation, S-sulfinylation, S-sulfonylation, succinylation, sulfation, glycation, carbamylation, carbonylation, biotinylation, carbamylation, pegylation, ISGylation, SUMOylation, ubiquitination, neddylation, pupylation, citrullination, deimination, deamidation, eliminylation, disulfide bridges, the covalent linkage of two cysteine amino acids, proteolytic cleavage, cleavage of a protein at a peptide bond, isoaspartate formation, racemization, and/or protein splicing.

Modifications may include the addition of heavy labels to molecules such as polypeptides. For example, polypeptides may be labeled with stable, non-radioactive isotopes or ‘heavy’ amino acids such as ¹²C by ¹³C (carbon-13), ¹⁴N by ¹⁵N (nitrogen-15), and ¹H by ²H (deuterium), preferably ¹³C₆¹⁵N₂-lysine and ¹³C₆¹⁵N₄-arginine. Such modification may be done by substitution of certain atoms (N, C, H; nitrogen, carbon, hydrogen) present in the molecule with a corresponding ‘heavy’ amino acid.

Modifications include amino acid additions, deletions or substitutions made relative to the reference sequence or portion thereof such as a wild-type sequence of a polypeptide. The modifications may be conservative or non-conservative amino acid substitutions. Where there are multiple modifications in a single polypeptide, the modifications in the polypeptide sequence may be a combination of conservative and non-conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in the Table below.

TABLE 4
Chemical properties of amino acids

| | | | |
|---|---|---|---|
| Ala (A) | aliphatic, hydrophobic, neutral | Met (M) | hydrophobic, neutral |
| Cys (C) | polar, hydrophobic, neutral | Asn (N) | polar, hydrophilic, neutral |
| Asp (D) | polar, hydrophilic, charged (−) | Pro (P) | hydrophobic, neutral |
| Glu (E) | polar, hydrophilic, charged (−) | Gln (Q) | polar, hydrophilic, neutral |
| Phe (F) | aromatic, hydrophobic, neutral | Arg (R) | polar, hydrophilic, charged |
| | | | (+) |
| Gly (G) | aliphatic, neutral | Ser (S) | polar, hydrophilic, neutral |
| His (H) | aromatic, polar, hydrophilic, | Thr (T) | polar, hydrophilic, neutral |
| | charged (+) | | |
| Ile (I) | aliphatic, hydrophobic, neutral | Val (V) | aliphatic, hydrophobic, |
| | | | neutral |
| Lys (K) | polar, hydrophilic, charged(+) | Trp (W) | aromatic, hydrophobic, |
| | | | neutral |
| Leu (L) | aliphatic, hydrophobic, neutral | Tyr (Y) | aromatic, polar, hydrophobic |

Where amino acids have similar polarity, this can be determined by reference to the hydropathy scale for amino acid side chains in the Table below.

TABLE 5
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Binding to a bait molecule Binding of a binding molecule to a bait molecule may be direct. For example direct binding is where a binding molecule binds to the bait molecule itself. The binding molecule may bind directly to the bait molecule via protein:protein interactions between amino acids of the binding molecule and amino acids of the bait molecule.

Binding of a binding molecule to a bait molecule may be indirect. For example, indirect binding may be where a binding molecule binds to the bait molecule via one or more other binding molecules, one or more of which of these binding molecules may bind directly or may indirectly bind to the bait molecule. The binding molecule may for example bind indirectly to the bait molecule via post-translational modifications on the binding molecule and/or the bait molecule. Post-translation modifications may include glycosylation, phosphorylation, acetylation and/or ubiquitination and others as disclosed herein.

### Binding Complexes

Binding complexes are formed when one or more binding molecules bind to a bait molecule. These binding complexes may comprise one or more of the binding complexes described herein which are in complex with any bait molecule described herein.

### Isolating Binding Complexes

Isolation of binding complexes may be carried out by any suitable means. A skilled person will readily recognise many possible ways of isolating the binding complexes.

Bait molecules may be bound to a surface prior to contact with the sample. For example, bait molecules may be bound to the surface of the well of a microplate and the sample comprising biological fluid which has been taken from the individual may be added to the well. Upon incubation, complexes between the bait molecule and binding molecule may form. In such an embodiment, removal of the sample comprising biological fluid following incubation and a step of washing of the complexes may be performed, thus isolating the complexes.

Bait molecules may be bound to a surface during the process of isolating the binding complexes. For example, bait molecules may be provided in a form which allows the bait molecules to bind to a surface during or after the step of capturing the one or more binding molecules. For example, bait molecules may be provided conjugated to biotin and immobilised using microparticles coated with streptavidin. Immobilisation could be achieved during or after the step of capturing the one or more binding molecules. In such an embodiment, removal of the sample comprising biological fluid following incubation and a step of washing of the complexes attached to microparticles may be performed, thus isolating the complexes. Any suitable alternative to microparticles could be used. Any suitable alternative to biotin and streptavidin could be used.

A automated liquid handling system may be used to isolate binding complexes.

### Identifying and Quantifying the One or More Binding Molecules

Identification and quantification of the one of more binding molecules may be carried out by any suitable means. A skilled person will readily recognise many possible ways of identifying and quantifying the one or more binding molecules.

Identification and quantification of the one or more binding molecules captured within a binding complex can be carried out by any suitable means. For example, a binding molecule may be identified and quantified using mass spectrometry.

Mass spectrometry that may be used to identify and/or quantify binding molecules include AMS (Accelerator Mass Spectrometry), Gas Chromatography-MS, LC-MS (Liquid Chromatography Mass Spectrometry), ICP-MS (Inductively Coupled Plasma-Mass spectrometry), IRMS (Isotope Ratio Mass Spectrometry), MALDI-TOF MS (Matrix Assisted Laser Desorption Ionisation-Time of Flight mass spectrometry), SELDI-TOF MS (Surface Enhanced Laser Desorption Ionization-Time of Flight mass spectrometry), Tandem Mass spectrometry (MS/MS), TIMS (Thermal Ionization-Mass Spectrometry), and SSMS (Spark Source Mass Spectrometry). Preferably, the type of mass spectrometry employed for identification and/or quantification of binding molecules is LC-MS/MS (Liquid Chromatography with tandem mass spectrometry).

Once binding complexes are formed and isolated, as described further herein, binding complexes may be processed by any suitable means. Preferably complexes are subjected to proteolytic digestion, as described herein, and the digestion products are then subjected to mass spectrometry techniques to identify and quantify the binding molecules. For example, where bait molecules are bound to the surface of the well of a microplate, following isolation of the binding complexes, as described herein, a solution comprising an agent capable of digesting proteins may be added to the well and incubated to form digestion products which will be released into the solution. The solution comprising released digestion products can then be removed from the well and subjected to mass spectrometry techniques to identify and quantify the binding molecules.

Preferably, identification and quantification of the one or more binding molecules is carried out in an automated manner.

### Quantification of Binding Molecules Using Bait Molecules

As explained further herein, the present targeted mass spectrometry-based assays can be used to quantify binding molecules in a biological fluid of an individual. By use of proteomics-based methods, such as LC-MS/MS, it is possible to quantify the amount of a binding molecule which is captured by a plurality of bait molecules from a sample comprising a biological fluid of an individual.

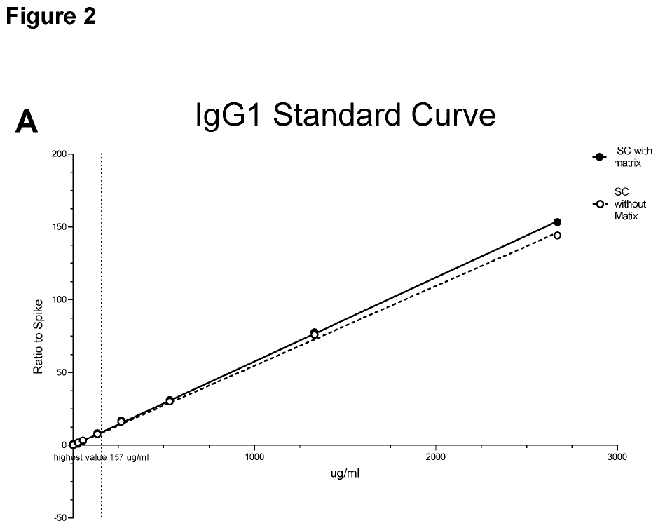

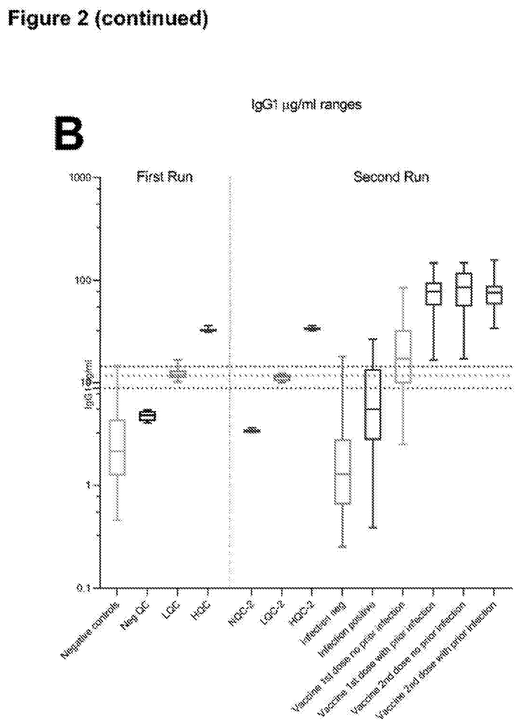

Quantification of proteins, peptides or other molecules by such proteomics-based methods is routine in the art. Standard curves can be established using reference standards of the analyte (binding protein) and these curves can be used to calibrate the system. Once an amount of analyte per unit volume of a reaction mixture is established using the proteomics platform of the invention, the standard curve can be used to extrapolate the amount of analyte present in the sample, and thus present in the biological fluid of the individual, can be calculated. By way of example, FIG. 2 shows a standard curve using IgG1. This curve demonstrates known quantities of protein bound to the column as part of an initial validation exercise. These data can subsequently be used to determine the amount of an unknown quantity of IgG1 binding molecule captured using a plurality of bait molecules in the assay of the invention.

Using such methods it is possible to calibrate a proteomics based system for the quantification of any desired binding molecule of interest, typically a protein, which is captured by a given bait molecule. The amounts of protein in a population of individuals can then be investigated. For example, the concentration of IgG1, or other protein of interest, in a population of individuals which have been vaccinated against SARS-CoV-2 can be established and compared with the concentration of IgG1 in a population of individuals which have been not vaccinated against SARS-CoV-2. The concentration of IgG1, or other protein of interest, which binds to e.g. spike proteins from different strains/variants of SARS-CoV-2 may provide information concerning whether a given individual has mounted an immune response against a given strain/variant of SARS-CoV-2 or whether the individual has mounted an immune response which might be effective against a given strain/variant of SARS-CoV-2.

As explained further herein, current gold standards for detecting and quantifying antibodies generated against SARS-CoV-2 are ELISA-based assays, such as the Roche Elecsys® Anti-SARS-CoV-2 assay, EDI Novel Coronavirus COVID-19 assay by Epitope Diagnostics and LIAISON SARS-CoV-2 assay by Diasorin. Each one of these assays uses a quantification mechanism measured in units per mL. This corresponds to units per mL of patient serum. The use of a quantification standard using units per mL provides a way of comparing quantification results across different platforms.

1 unit of e.g. IgG1 per mL of serum may be defined as the equivalent reactivity in a given assay seen by 1 μg/mL of a reference standard of IgG1 (see e.g. Garcia-Beltran, W. F., et al. 2021. Cell. 184, pp 476-488, page e4 under the heading “SARS-CoV-2 receptor binding domain and spike IgG, IgM, and IgA ELISA”).

In any of the methods of the invention described or defined herein the concentration of a given binding protein which is captured by bait molecules in the present system may be expressed in units per mL (U/mL), e.g. U/mL of patient serum or plasma or in μg/mL, e.g. μg/mL of patient serum or plasma.

In any of the methods of the invention described or defined herein the concentration of IgG antibodies/immunoglobulins which are captured by bait molecules in the present system can be expressed in units per mL (U/mL), e.g. U/mL of patient serum or in μg/mL, e.g. μg/mL of patient serum or plasma.

In any of the methods of the invention described or defined herein the concentration of IgG1 antibodies/immunoglobulins which are captured by bait molecules in the present system can be expressed in units per mL (U/mL), e.g. U/mL of patient serum or in μg/mL, e.g. μg/mL of patient serum or plasma.

In any of the methods of the invention described or defined herein the concentration of antibodies/immunoglobulins of IgG, IgA, and IgM isotype may be quantified according to international units (IU) per volume, such as IU per mL, e.g. IU per mL of patient serum or plasma.

The World Health Organiszation (WHO) Expert Committee on Biological Standardization defined international units (IU) for the quantitative determinations of IgG, IgA, and IgM. In each case these international units were each defined as the activity contained in 0.8147 mg of the respective “International Reference Preparation” of IgG, IgA, and IgM. The International Reference Preparations were established as the British Research Standard for Human Serum Immunoglobulins G, A, and M (see Rowe, D. S., Grab. B, and Andersen, S. G (1972) An International Reference Preparation for Human Serum Immunoglobulins G, A and M. Content of Immunoglobulins by Weight. Bull. Wld. Hlth. Org. 46, 67.).

Whilst an international unit is defined by potency, it may nevertheless be used as a measure of concentration in accordance with any of the methods described and defined herein. Accordingly, the term “concentration” as used herein may mean units (U) per volume, such as U per mL, or international units (IU) per volume, such as IU per mL, or amounts per volume such as μg per mL.

In any of the methods described and defined herein the measure of the binding molecule of interest may be expressed as a molar ratio of the amount of binding molecule to bait.

### Correlation Between SARS-CoV-2 Immune Response and IgG Concentration

Manufacturers of the Roche Elecsys® Anti-SARS-CoV-2 assay have established threshold values of IgG antibodies in U/mL which have been shown to correlate with immune responses to SARS-CoV-2. Thus a concentration of 0.8 U/mL or more of IgG in patient serum has been shown in the Roche Elecsys® assay to indicate that the patient has produced IgG antibodies which bind to the SARS-CoV-2 S1 receptor binding domain, thus demonstrating that the individual has likely mounted an immune response to SARS-CoV-2. A concentration of less than 0.8 U/mL of IgG in patient serum has been shown in the Roche Elecsys® assay to indicate that the patient does not have circulating IgG antibodies which bind to the SARS-CoV-2 S1 receptor binding domain, thus demonstrating that the individual has likely not mounted an immune response to SARS-CoV-2, or else the patient's IgG response has waned back to basal levels. A concentration of 2500 U/mL or more of IgG in patient serum has been shown in the Roche Elecsys® assay to correlate with neutralising antibodies.

Accordingly, in any of the methods of the invention described or defined herein, when the concentration of IgG antibodies in the individual is 0.8 U/mL or more the individual is classified as having produced IgG antibodies to SARS-CoV-2 and thereby is classified as having mounted an immune response to SARS-CoV-2. In any of the methods of the invention described or defined herein, when the concentration of IgG antibodies in the individual is a number of U/mL or a number of μg/mL or a number of IU/mL which equates with 0.8 U/mL or more as determined in the Roche Elecsys® assay the individual is classified as having produced IgG antibodies to SARS-CoV-2 and thereby is classified as having mounted an immune response to SARS-CoV-2. The binding molecules which are identified and quantified may be all antibodies of IgG isotype. The binding molecules which are identified and quantified may be antibodies of any one or more of isotypes IgG1, IgG2, IgG3 and IgG4. Preferably the biological fluid of the individual is plasma or serum.

In any of the methods of the invention described or defined herein, when the concentration of IgG antibodies in the individual is a number of U/mL or a number of μg/mL or a number of IU/mL which equates with 2500 U/mL or more as determined in the Roche Elecsys® assay the individual is classified as having an IgG antibody concentration which causes inhibition of the biological activity of SARS-CoV-2 and wherein the individual is thereby classified as having generated a neutralising antibody response to SARS-CoV-2, preferably wherein the biological activity of SARS-CoV-2 is the ability of SARS-CoV-2 to infect a human cell expressing the ACE2 receptor. The binding molecules which are identified and quantified may be all antibodies of IgG isotype. The binding molecules which are identified and quantified may be antibodies of any one or more of isotypes IgG1, IgG2, IgG3 and IgG4. Preferably the biological fluid of the individual is plasma or serum.

### Statistical Parameters

The assay methods of the invention may be used to determine whether or not the concentration of an analyte in the biological fluid of an individual is statistically significantly increased compared to the concentration of the analyte in a control.

What constitutes a control is further described and defined herein. The concentration of the analyte in a control may be the average concentration of the analyte in a population of individuals of a common group, for example the average concentration of the analyte in a population of individuals which have not been vaccinated against SARS-CoV-2 and which have not been infected with SARS-CoV-2. The term “average” may mean any suitable appropriate measure of the concentration of the analyte in a common group, such as the mean, median or mode. The average may be expressed as the median.

The term “statistically significant” is a term of art. By “statistically significant” it is meant that the observed change is caused by something other than chance (which may be a “false positive”). Statistical significance can be determined by any method known in the art. Statistical significance may be determined by the p value. The p-value is a measure of the probability that a difference between groups or observations will occur by chance alone. The lower the p-value, the less likely it is that a difference was caused by chance. For example, a p-value of 0.01 means that the result is expected to happen only once in 100 times. Such a result is therefore highly unlikely to occur by chance alone, and is therefore considered to be highly significant. In any of the methods described and defined herein a statistically significant result is one where the p-value is 0.05 or less. Preferably, the p-value is 0.04 or less, 0.03 or less, 0.02 or less, 0.01 or less, 0.005 or less or 0.001 or less.

The assays of the invention may be defined using standard receiver operating characteristic (ROC) statistical analysis. The assays of the invention can be defined according to parameters relating to their statistical specificity and sensitivity. These parameters define the likelihood of false positive and false negative test results. The lower the proportion of false positive and false negative test results the more statistically accurate the assay becomes. In ROC analysis 100% sensitivity corresponds to a finding of no false negatives, and 100% specificity corresponds to a finding of no false positives.

In any of the methods of the invention described or defined herein, the ROC sensitivity may be 90% or greater, and the ROC specificity may be 90% or greater, 91% or greater, 92% or greater, 93% or greater, 94% or greater, 95% or greater, 96% or greater, 97% or greater, 98% or greater, 99% or 100%.

The ROC sensitivity may be 91% or greater, and the ROC specificity may be 91% or greater, 92% or greater, 93% or greater, 94% or greater, 95% or greater, 96% or greater, 97% or greater, 98% or greater, 99% or 100%.

The ROC sensitivity may be 92% or greater, and the ROC specificity may be 92% or greater, 93% or greater, 94% or greater, 95% or greater, 96% or greater, 97% or greater, 98% or greater, 99% or 100%.

The ROC sensitivity may be 93% or greater, and the ROC specificity may be 93% or greater, 94% or greater, 95% or greater, 96% or greater, 97% or greater, 98% or greater, 99% or 100%.

The ROC sensitivity may be 94% or greater, and the ROC specificity may be 94% or greater, 95% or greater, 96% or greater, 97% or greater, 98% or greater, 99% or 100%.

The ROC sensitivity may be 95% or greater, and the ROC specificity may be 95% or greater, 96% or greater, 97% or greater, 98% or greater, 99% or 100%.

The ROC sensitivity may be 96% or greater, and the ROC specificity may be 96% or greater, 97% or greater, 98% or greater, 99% or 100%.

The ROC sensitivity may be 97% or greater, and the ROC specificity may be 97% or greater, 98% or greater, 99% or 100%.

The ROC sensitivity may be 98% or greater, and the ROC specificity may be 98%, 99% or 100%.

The ROC sensitivity may be 99%, and the ROC specificity may be 99% or 100%.

The ROC sensitivity may be 100%, and the ROC specificity may be 100%.

In any of the methods of the invention described or defined herein, the method may exhibit a sensitivity of 98.8% and a specificity of 100%.

In any of the methods of the invention described or defined herein, the method may exhibit a sensitivity of 98.8% (95% CI: 98.1-99.3%) and a specificity of 100% (95% CI: 99.7-100%).

### Digestion Step

A digestion step may be performed using any suitable agent capable of cleaving proteins. For example, the agent may be any suitable enzyme. The enzyme may be trypsin, chymotrypsin, LysC, LysN, AspN, GluC and ArgC.

### Surface Binding of Bait/Binding Molecule Complexes

### Microplate

Any of the methods of the invention may be performed using microplates for the processing of samples.

Microplates to be used in the invention may be any microplate or microtiter plate suitable for adhering one or more bait molecules. For example, a microplate may be a 96-well, 384-well or 1536-well microtitre plate which can be obtained from any suitable commercial supplier.

### Microparticle

Any of the methods of the invention may be performed using microparticles or microbeads for the processing of samples.

Microparticles to be used in the invention may be any microparticle used in the field. For example, the microparticle may be magnetic. The microparticle may be coated with any surface attachment molecule that allows for surface attachment and hence isolation of the bait molecule and any binding complexes.

For example, the microparticle coated with a surface attachment molecule may bind to a bait molecule modified with a complementary surface attachment molecule. A system involving biotin/streptavidin may be used, or any suitable alternative, as described further herein. The microparticle may then be to captured onto a surface thus allowing the bait molecule and binding complexes to be isolated. For instance, the microparticle may be magnetic allowing the microparticle and the associated bait molecule and binding complexes to be captured onto the surface of an electrode.

### Surface Attachment Molecules

The bait molecule used in methods described herein may be modified with one or more surface attachment molecules. Such surface attachment molecules will typically be attached chemically, they may also be attached to surfaces by indirect means such as via affinity interactions. For example, the bait molecules may be modified with biotin and bound to surfaces coated with avidin or streptavidin.

For the immobilization of molecules to surfaces (e.g. planar surfaces), microparticles and beads etc., a variety of surface attachment methods and chemistries are available. Surfaces may be functionalised or derivatized to facilitate attachment. Such functionalisations are known in the art. For example, a surface may be functionalised with a polyhistidine-tag (hexa histidine-tag, 6×His-tag, His6 tag or His-tag®), Ni-NTA, streptavidin, biotin, an oligonucleotide, a polynucleotide (such as DNA, RNA, PNA, GNA, TNA or LNA), carboxyl groups, quaternary amine groups, thiol groups, azide groups, alkyne groups, DIBO, lipid, FLAG-tag (FLAG octapeptide), polynucleotide binding proteins, peptides, proteins, antibodies or antibody fragments. The surface may be functionalised with a molecule or group which specifically binds to the bait molecule.

### Kits

A kit of the invention may comprise any one or more of the following:
a. one or more vials each comprising a plurality of identical isolated bait molecules for use in capturing one or more binding molecules from a sample comprising biological fluid which has been taken from an individual;
b. at least one microplate and/or one or more vials each comprising microparticles for use in isolating binding complexes between the bait molecules and the one or more binding molecules;
c. one or more vials each comprising a plurality of identical molecules for use as a reagent blank, optionally wherein at least one vial comprises molecules of bovine serum albumin or casein, preferably wherein at least one vial comprises molecules of myoglobin, optionally horse myoglobin;
d. one or more vials each comprising a dilution buffer for use in diluting biological fluid which has been taken from an individual, optionally phosphate-buffered saline (PBS);
e. one or more vials comprising a wash buffer for use in washing binding complexes between the bait molecules and the one or more binding molecules, optionally phosphate-buffered saline (PBS);
f. one or more vials comprising a surfactant for use in solubilising proteins;
g. one or more vials comprising a digestion buffer comprising an enzyme capable of cleaving proteins, preferably wherein the enzyme is a protease; and
h. one or more vials comprising a plurality of identical molecules for use as an internal calibration standard when performing quantification of a protein by mass spectrometry, preferably wherein the molecules are peptides.

In any such kit the dilution buffer may be any suitable buffer such as any suitable non-denaturing isotonic buffer.

In any such kit the one or more vials each comprising a plurality of identical isolated bait molecules may comprise any pathogen polypeptide; any autoantigen polypeptide; any neoantigen polypeptide or any polypeptide of a gene therapy or vaccine delivery vector, such as a polypeptide of a viral vector e.g. an adenoviral vector, preferably AAV.

In any such kit the one or more vials each comprising a plurality of identical isolated bait molecules may comprise any viral polypeptide, subunit or any fragment thereof as described or defined herein.

In any such kit the one or more vials comprising a surfactant for use in solubilising proteins may comprises a surfactant which is:
a. sodium 3-[(2-methyl-2-undecyl-1,3-dioxolan-4-yl)methoxy]-1-propanesulfonate,
b. 3-[N,N-Dimethyl(3-myristoylaminopropyl)ammonio]propanesulfonate (amidosulfobetaine-14) or
c. 3, 12-α-Dihydroxy-5β-cholan-24-oic acid monosodium salt (sodium deoxycholate).

In any such kit the one or more vials comprising a digestion buffer comprising an enzyme capable of cleaving proteins may comprise a protease which is:
a. chymotrypsin,
b. LysC,
c. LysN,
d. AspN,
e. GluC, or
f. ArgC.

In any such kit the one or more vials comprising a plurality of identical molecules for use as an internal calibration standard may comprises a plurality of identical heavy labelled peptides, preferably wherein the peptides are labelled using ¹³C₆¹⁵N₂-lysine and ¹³C₆¹⁵N₄-arginine. The plurality of identical heavy labelled peptides may have an amino acid sequence which is identical to an amino sequence of any pathogen polypeptide, any autoantigen polypeptide, any neoantigen polypeptide, any polypeptide of a gene therapy or vaccine delivery vector, any virion, any gene therapy or vaccine delivery vector, or any viral polypeptide, subunit or any fragment thereof as defined and described herein Any such kit may comprise one or more vials each comprising a plurality of identical monoclonal antibody species, wherein antibodies of a vial may bind to an amino acid sequence which is common between two, more or all of:
a. wild-type SARS-CoV-2;
b. a variant of wild-type SARS-CoV-2, wherein the amino acid sequence of the variant differs from the amino acid sequence of wild-type by one or more amino acid mutations, optionally wherein one or more amino acid mutations are in an envelope protein or wherein one or more amino acid mutations are in a nucleocapsid protein, preferably wherein one or more amino acid mutations are in the spike protein;
c. SARS-CoV-2 B.1.1.7 variant (alpha variant);
d. SARS-CoV-2 B.1.351 variant (beta variant);
e. SARS-CoV-2 P.1 variant (gamma variant);
f. SARS-CoV-2 B.1.617.2 variant (delta variant);
g. SARS-CoV-2 B.1.427/B.1.429 variant (epsilon variant);
h. SARS-CoV-2 P.2 variant (zeta variant);
i. SARS-CoV-2 B.1.525 variant (eta variant);
j. SARS-CoV-2 P.3 variant (theta variant);
k. SARS-CoV-2 B.1.526 variant (Iota variant);
l. SARS-CoV-2 B.1.617.1 variant (kappa variant);
m. SARS-CoV-2 C.37 variant (lambda variant).
and wherein the amino acid sequence is a sequence of (i) the full-length coronavirus spike protein, subunit, RBD or any fragment thereof; (ii) the full-length coronavirus envelope protein or a fragment thereof; (iii) the full-length coronavirus membrane protein or a fragment thereof; or (iv) the full-length coronavirus nucleocapsid protein or a fragment thereof.

### EXAMPLES

The following Examples are provided to illustrate the invention, but not to limit the invention.

### Example 1—Materials and Methods

### Ethics Statement

A total of 141 human sera were obtained from the COVIDsortium Healthcare Workers bioresource which is approved by the ethical committee of UK National Research Ethics Service (20/SC/0149) and registered on ClinicalTrials.gov (NCT04318314). The study conformed to the principles of the Helsinki Declaration, and all subjects gave written informed consent.

### COVIDsortium Healthcare Worker Participants

WT SARS-CoV-2 infection of study participants was determined using baseline and weekly nasal RNA stabilizing swabs and Roche Cobas® SARS-CoV-2 reverse transcriptase polymerase chain reaction (RT-PCR) test as well as baseline and weekly antibody testing using the spike protein S1 IgG EUROIMMUN enzyme-linked immunosorbent assay (ELISA) and nucleocapsid ROCHE Elecsys electrochemiluminescence immunoassay (ECLIA). Antibody ratios >1.1 were considered test positive for the EUROIMMUN SARS-CoV-2 ELISA and >1 was considered test positive for the ROCHE Elecsys anti-SARS-CoV-2 ECLIA following Public Health England evaluation (Public Health England, COVID-19: laboratory evaluations of serological assays, gov.uk, 16 Mar. 2021).

The cross-sectional, case-controlled vaccine sub-study discussed here (n=51) collected samples at a mean/median timepoint of 22d (±2d SD) after administration of the first dose of the mRNA vaccine, BNT162b2. This vaccine sub-study recruited health care workers (HCW) previously enrolled in the 16-18 week sub-study (Liew, F. Y. et al. Eur J Immunol 1984). It included 25 HCW (mean age 44 yr, 60% male) with previous laboratory defined evidence of WT SARS-CoV-2 infection and twenty-six HCW (mean age 41y, 54% male) with no laboratory evidence of SARS-CoV-2 infection throughout the initial 16-week longitudinal follow up.

Neutralising antibody and RBD ELISA data obtained by ROCHE Elecsys anti-SARS-CoV-2 ECLIA following Public Health England (PHE) has been previously published. (Reynolds, C. J. et al. Science 2021).

### SARS2-CoV-2 Immunocomplex Assay—Sample Preparation

Ninety six well microtiter plates (Waters PLC) were coated with either Wuhan Hu-1 SARS-CoV-2 spike protein (S1) (Genscript Z03501), alpha B.1.1.7 SARS-CoV-2 (2019-nCoV) Spike S1 (HV69-70 deletion, N501Y, D614G)-His Recombinant Protein (Sino biological 40591-V08H7) or B.1.1351 SARS-CoV-2 Spike protein (S1, E484K, K417N, N501Y, His Tag (Genscript Z03531-1). S1 protein was diluted to 150 μg/ml in PBS and 10 μl added to the bottom of each well. Wells were topped with 140 μl of Carbonate/Bicarbonate buffer 100 mM at pH 9.6 and then incubated for 12-16 hours at 4° C. All further incubations were performed at room temperature (RT). Plates were washed with 200 μl of PBS and then incubated for 1 hr with 200 μl of 1 mg/ml horse myoglobin (Sigma UK) solution in PBS followed by 3 washes with PBS. Plate wells were stored with PBS and kept at 4° C. until used.

### Serum

Serum samples were diluted 1:10 in 0.05 mg/ml horse myoglobin and added to S1 coated wells for 1 hr.

### Dried Blood Spots

6 mm DBS spots were punched into a 2 ml micro tube and extracted using 175 μl of 0.05 mg/ml Horse myoglobin solution in PBS for 1 hour on the shaker. Samples were centrifuged at max rpm on benchtop centrifuge for 10 min. An aliquot 150 μl per reaction was added to the baited well for 1 hr.

### Dried Saliva Spot (Lollipop)

6 mm saliva spots were punched into a 2 ml micro tube and extracted using 250 μl of 0.05 mg/ml Horse myoglobin solution in PBS for 1 hour on the shaker. Samples were centrifuged at max rpm on benchtop centrifuge for 10 min. An aliquot of 150 μl per reaction was added to the baited well for 1 hr.

Sample (serum, DBS or Saliva spot) was removed and wells were washed once with 200 μl 0.05% Tween 20 in PBS and then 3 times with PBS. Bound proteins was then digested using ProteinWorks eXpress digest kit (Waters, Manchester UK) as per manufacturers protocol.

### Label Free Proteomics Analysis

Digested samples were desalted using solid phase extraction and label free proteomics analysis was performed as previously described (Bliss, E. et al. Biol Proced Online 2016).

### Targeted LC-MS/MS Analysis

Digested samples were injected onto a Waters 50 mm UPLC Premier® C18 1.6 μm column operating at 45° C., for chromatographic separation. Mobile phase A consisted of: 0.1% formic acid in water and B: 0.1% formic acid in ACN, pumped at a flow rate of 0.6 mL min-1. The starting conditions of 3% B were kept static for 0.1 minutes, before initialising the linear gradient to elute and separate peptides over 7.7 minutes to 40% B. B was linearly increased to 80% over 0.2 minutes and held for 1 minute to wash the column before returning to the initial conditions followed by equilibration for 1 minute prior to the subsequent injection. The LC system was coupled to a Waters Xevo-TQ-S triple quadrupole mass spectrometer for multiple reaction monitoring (MRM) detection in positive electrospray ionisation mode. The capillary voltage was set to 2.8 kV, the source temperature to 150° C., the desolvation temperature to 600° C., the cone gas and desolvation gas flows to 150 and 1000 L hour-1 respectively. The collision gas consisted of nitrogen and was set to 0.15 mL min-1. The nebuliser operated at 7 bar. The cone energy was set to 35 V and the collision energies varied depending on the optimal settings for each peptide.

### Data Analysis

Raw data for label free proteomics analysis was analysed using ProteinLynx Global Server v3.0 (Waters, UK). Data were searched against a uniprot reference human proteome with SARS-CoV-2 S1 protein sequence added set to a 1% FDR, one missed cleavages with fixed modification of carboamidomethylation of cysteines and dynamic modifications of deamidation of asparagine/glutamine, and oxidation of methionine.

Raw LC-MS/MS data was analysed using Skyline open source software (https://skyline.ms/project/home/software/Skyline/begin.view). Peptide identifications were determined from prior analysis of digested serum and immunoglobulin standards (Invitrogen) by a minimum of 6 transitions and matched to in-silico spectral library (Prosit) for additional confirmation. Two optimal transitions were used for final MRM analysis. Peptide abundance data were normalised to S1 peptide QIAPGQTGK. For comparison analysis between variant assays data were normalised by Z-Score. Exported data were analysed using Microsoft Excel, Graphpad Prism v9 and SIMCA v.15 (Umetrics Sweden).

TABLE 6
peptides included in the targeted multiplex assay.

| | | | Sequence used in |
|---|---|---|---|
| Function | Accession | Description | Multiplex |
| Immune | P01011 | Alpha-1- | ITLLSALVETR (SEQ |
| function | | Antichymotrypsin | ID NO: 20) |
| related | | | LINDYVK (SEQ ID |
| proteins | | | NO: 21) |
| | P01023 | alpha-2- | GPTQEFK (SEQ ID |
| | | Macroglobulin | NO: 22) |
| | | | VGFYESDVMGR (SEQ |
| | | | ID NO: 23) |
| | P08697 | alpha-2- | LGNQEPGGQTALK |
| | | antiplasmin | (SEQ ID NO: 24) |
| | P01019 | Angiotensinogen | ALQDQLVLVAAK |
| | | | (SEQ ID NO: 25) |
| | | | DPTFIPAPIQAK |
| | | | (SEQ ID NO: 26) |
| | P02647 | Apoliporotein A1 | LSPLGEEMR (SEQ ID |
| | | | NO: 27) |
| | P02656 | Apoliporotein C3 | DALSSVQESQVAQQA |
| | | | R (SEQ ID NO: 28) |
| | P06396 | Gelsolin | AVEVLPK (SEQ ID |
| | | | NO: 29) |
| | | | GASQAGAPQGR (SEQ |
| | | | ID NO: 30) |
| | P19827 | Inter-Alpha- | LDAQASFLPK (SEQ |
| | | Trypsin | ID NO: 31) |
| | | Inhibitor | VTYDVSR (SEQ ID |
| | | Heavy Chain 1 | NO: 32) |
| | P35542 | Serum Amyloid | GPGGVWAAK (SEQ ID |
| | | A4 | NO: 33) |
| | P05546 | Heparin | TLEAQLTPR (SEQ ID |
| | | Cofactor 2 | NO: 34) |
| | | | VSMMQTK (SEQ ID |
| | | | NO: 35) |
| | P02766 | Transthyretin | AADDTWEPFASGK |
| | | | (SEQ ID NO: 36) |
| Antiviral | Q6FI13 | Histone H2A | AGLQFPVGR (SEQ ID |
| histones | | | NO: 37) |
| | P62807 | Histone H2B | LLLPGELAK(SEQ ID |
| | | | NO: 38) |
| Complement | P0C0L5; | Complement | LELSVDGAK (SEQ ID |
| | P0C0L4 | factor | NO: 39) |
| | | C4 a and b | VGDTLNLNLR (SEQ |
| | | | ID NO: 40) |
| | P04003 | Complement | LSLEIEQLELQR (SEQ |
| | | factor | ID NO: 41) |
| | | C4 binding | TWYPEVPK (SEQ ID |
| | | protein A | NO: 42) |
| | | | YTCLPGYVR (SEQ ID |
| | | | NO: 62) |
| | P01024 | Complement C3 | AEDLVGK (SEQ ID |
| | | | NO: 43) |
| | P02745 | Complement C1q | SLGFCDTTNK (SEQ |
| | | subcomponent | ID NO: 44) |
| | | subunit A | |
| | P02747 | Complement C1q | FQSVFTVTR (SEQ ID |
| | | subcomponent | NO: 45) |
| | | subunit C | |
| | P02746 | Complement C1q | GNLCVNLMR (SEQ ID |
| | | subcomponent | NO: 46) |
| | | subunit B | |
| | ??? P00751 | Complement | DISEVVTPR (SEQ ID |
| | | Factor B | NO: 63) |
| | | Fragment b | |
| | | protein | |
| Membrane | P01031 | Complement | GIYGTISR (SEQ ID |
| Attack | | factor C5a | NO: 47) |
| complex | | | |
| | P01031 | Complement | VFQFLEK (SEQ ID |
| | | factor C5b | NO: 48) |
| | P07360 | Complement | SLPVSDSVLSGFEQR |
| | | factor C8 | (SEQ ID NO: 49) |
| | P02748 | Complement | LSPIYNLVPVK (SEQ |
| | | factor C9 | ID NO: 50) |
| | P07357 | Complement | MESLGITSR (SEQ ID |
| | | factor C8a | NO: 64) |
| Soluble | P04004 | Vitronectin | FEDGVLDPDYPR |
| membrane | | | (SEQ ID NO: 51) |
| attack | | | |
| complex | | | |
| | P27169 | Parao xonase 1 | IFFYDSENPPASEVLR |
| | | | (SEQ ID NO: 52) |
| | | | IQNILTEEPK (SEQ |
| | | | ID NO: 53) |
| | P10909 | Clusterin | LFDSDPITVTVPVEVS |
| | | | R (SEQ ID NO: 54) |
| Platelet | P62834 | Ras-Related | LVVLGSGGVGK (SEQ |
| protein | | Protein Rap-1A | ID NO: 55) |
| | | IgG Common | |
| Immuno- | P0DOX5 | Total IgG | NQVSLTCLVK (SEQ |
| globulins | P01860, | | ID NO: 65) |
| | P01857, | | |
| | P01861, | | |
| | A0A286YEY4 | | |
| | P01857 | Immunoglobulin | GPSVFPLAPSSK (SEQ |
| | | heavy constant | ID NO: 56) |
| | | gamma 1 | |
| | P01859 | Immunoglobulin | GLPAPIEK (SEQ ID |
| | | heavy constant | NO: 57) |
| | | gamma 2 | |
| | P01860 | Immunoglobulin | TPLGDTTHTCPR (SEQ |
| | | heavy constant | ID NO: 58) |
| | | gamma 3 | |
| | P01876 | Immunoglobulin | TPLTATLSK (SEQ ID |
| | | heavy constant | NO: 59) |
| | | alpha 1 | |
| | P01871 | Immunoglobulin | GFPSVLR (SEQ ID |
| | | heavy constant | NO: 60 |
| | | mu | |
| | P01861 | Immunoglobulin | GLPSSIEK (SEQ ID |
| | | heavy constant | NO: 61) |
| | | gamma 4 | |
| | P0DOX7 | Immunoglobulin | VDNALQSGNSQESVT |
| | P01834 | kappa light | EQDSK (SEQ ID NO: |
| | | chain | 66) |
| | P0DOX8, | Immunoglobulin | YAASSYLSLTPEQWK |
| | B9A064; | lamda light | (SEQ ID NO: 67) |
| | A0M8Q6; | chain | |
| | P0CF74; | | |
| | P0DOY3; | | |
| | P0DOY2; | | |
| | P0CG04 | | |
| SARS-COV2 | P0DTC2 | SARS-COV2 spike | GVYYPDK (SEQ ID |
| spike | | | NO: 68) |
| bait | | | |
| antigen | | | |
| | P0DTC2 | SARS-COV2 spike | FASVYAWNR (SEQ ID |
| | | | NO: 69) |
| | P0DTC2 | SARS-COV2 spike | VGGNYNYLYR (SEQ |
| | | | ID NO: 70) |
| | P0DTC2 | SARS-COV2 spike | QIAPGQTGK (SEQ ID |
| | | | NO: 71) |

### Example 2—Sensitivity and Specificity of the Targeted Mass Spectrometry-Based Assay

A standard curve for IgG1 (the main immunoglobulin generated against SARS-CoV-2) using a commercial IgG protein standard was created in a replica matrix. Good linearity (R>0.99) was observed up to 2500 fmol (FIG. 2A). The highest sample value observed was 960 fmoles. Therefore linearity is well within the highest detectable values without dilution. For the lowest point, 1 fmole is the lowest value with an acceptable relative standard deviation (coefficient of variation). Using blank samples (positive QC sample, no bait antigen) the limit of detection (LOD) and quantitation (LOQ) were determined to be 19.2 fmol (97.08 ng/ml) and 20.88 (105.57 ng/ml) respectively.

Inter and intra assay batch variation were determined using a positive sample as a high QC which was diluted 10 fold to create a low QC. Inter-batch CV for low QC 41.96 fmol is 10.43% and high QC 77.94 fmol is 5.35%. Intrabatch QC was determined over 2 separate analysis runs to be <20%.

Specificity was determined by analysis against the Wuhan-Hu1 S1 protein using 100 negative plasma samples taken before 2020 before SARS-CoV-2 emerged (FIG. 2B). A negative serum QC was included in the analysis. A cut-off of 3 standard deviations (3×4.96 fmol) above the mean (24.31 fmol) was determined as 39.15 fmol. The negative serum QC value was determined as 35.5 fmol which is therefore below the cut-off for a negative sample. 2 samples in the negative sample cohort analysis were above the cut-off at 43 and 43.88 fmol. This gives the assay 98% specificity which is reported for many other SARS-CoV-2 ELISA-based assays.

### Example 3—Correlation Between Targeted Mass Spectrometry-Based Assay and Roche Elecsys® Anti-SARS-CoV-2 S

The inventors have developed a quantitative assay for the simultaneous measurement of Severe acute respiratory syndrome coronavirus 2 (SARS-CoV-2) specific immunocomplex proteins, present in various biological fluids (saliva, ‘saliva lollipops’, dried bloodspots, whole blood and serum/plasma). By using a ‘bait’ and targeted proteomic approach. They immobilised SARS-CoV-2 spike S1 protein or nucleocapsid to a 96-well plate, incubated with various biological fluids and using a mass spectrometry based method (LC-MS/MS) directly measured those proteins that bound to the virus protein e.g. antibodies, antibody-complexes, and proteins with a high affinity for the virus. Previous proteomic analyses of proteins binding to the bait allowed the inventors to identify proteins, which they designated ‘the immunocovidome’. This panel of proteins has been developed into a targeted proteomic assay that can analyse up to 20 proteins in <8 minutes in an LC-MS/MS run. Results from such run can be given as the molar ratio of immuno-captured proteins to SARS-CoV-2 S1 or nucleocapsid.

Using this assay, the inventors explored the use of various S1 spike proteins from the Wuhan Hu-1, alpha (B.1.1.7) and beta (B.1.351) strains as different baits to evaluate the binding to immune-based complexes present in the serum of patients of a) unexposed (no vaccine or infection) versus b) vaccinated and previous infection positive (PCR-positive for SARS-CoV-2 and c) no prior SARS-CoV-2 infection. Results of these analyses were compared with previously published data obtained using the Roche Elecsys® Anti-SARS-CoV-2 S, microneutralization assays.

The results demonstrated a good correlation of LC-MS/MS IgG1 data with Roche Elecsys® Anti-SARS-CoV-2 S data (r²>0.9) and neutralising antibody levels. Significantly altered levels of IgG3, IgA and IgM were detected. An altered complement protein response between strains with increased complement against the alpha and beta strains but not the original Wuhan-hu1 was also observed. This result indicated higher non-neutralising antibody activity against the variant strains. Paired analyses of dried bloodspots and saliva spots over a 2 week post vaccination period showed good correlation and an increase of IgG1 in blood and saliva 5-6 days after vaccination.

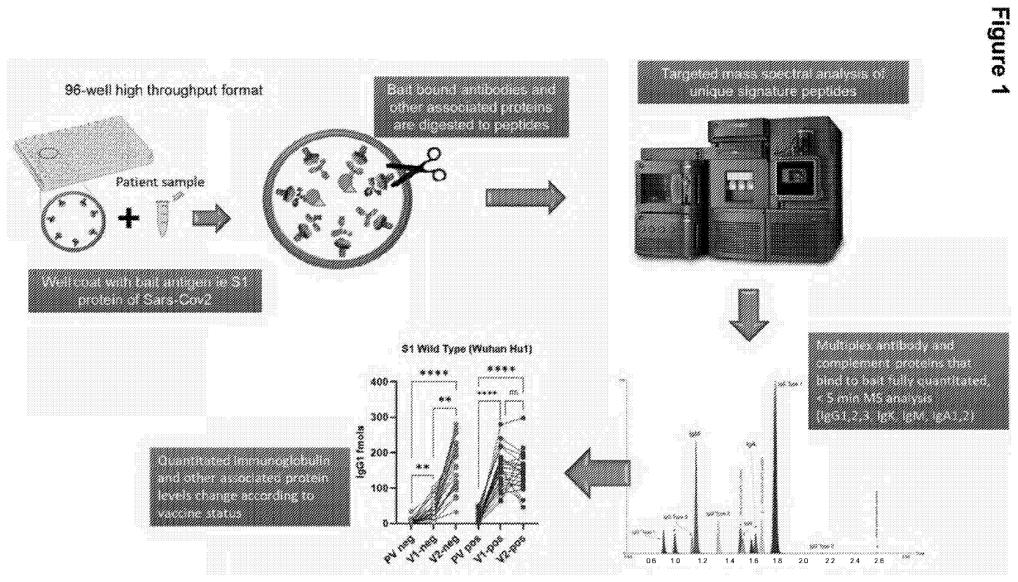

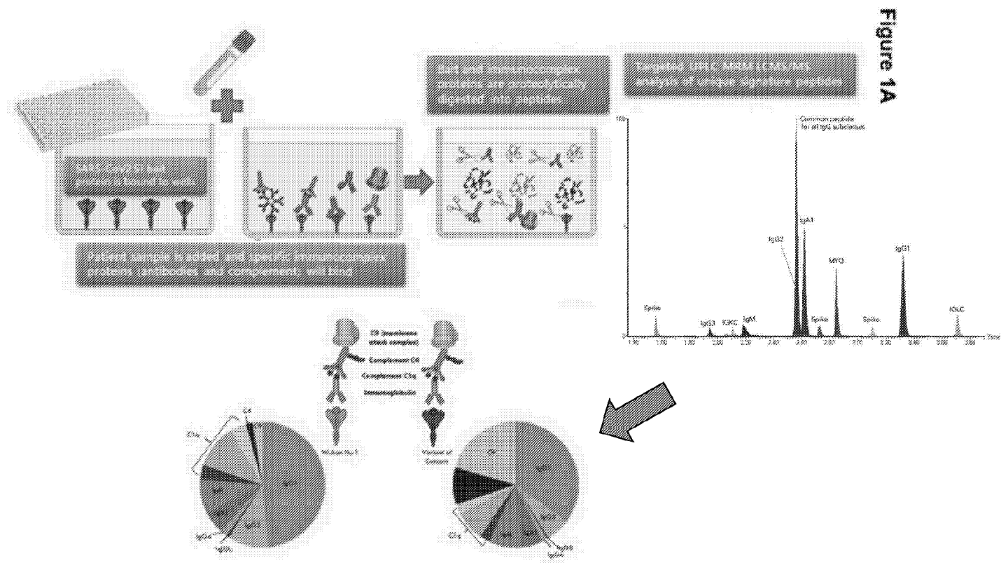

The inventors have developed a targeted proteomic multiplex assay that can use a bait molecule such as the spike S1 or the nucleocapsid protein of SARS-CoV-2 for the detection and measurement of proteins that form an immunocomplex against the SA RS-Cov-2SARS-CoV-2 S1 spike protein. Similar to an ELISA-based assay multiwell plates are coated with a commercially available SARS-CoV-2 S1 protein and blocked with a protein that causes minimal interference in LC-MS/MS analysis (see FIG. 1).

After sample incubation, detection is achieved by direct tryptic digestion of the nucleocapsid- or the S1 protein-immunocomplex, followed by LC-MS/MS analyses. Using this method, the inventors detected and quantified all molecules shown to bind to the ‘bait molecule(s)’ including antibodies, antibody-associated proteins and proteins with an affinity for SARS-CoV-2. These molecules were identified previously through proteomic profiling and/or use of the literature. This multiplex of proteins included the individual subtypes of IgG (1-3), IgM and IgA (1-2). Complement proteins associated with antibody-mediated complement response and membrane attack complex were included as well as immunological proteins with an undefined association. A complete list of these proteins identified and included in the assay are shown in Table 7.

Table 7 provides a list of the proteins included in the multiplexed assay
that are quantitated. This list of proteins or ‘covidome’ include
those proteins which have been discovered using proteomics analyses or
are known to associated with, and be involved in, a SARS-CoV-2 immune response.

| Associated Immune function | Protein | Description |
|---|---|---|
| Immune response | GSN | Gelsolin |
| associated proteins | TTHY | Transthyretin |
| | SERPIND1 | Heparin Cofactor 2 |
| | SAA4 | Serum Amyloid A4 |
| | AACT | Alpha-1-Antichymotrypsin |
| | A2M | alpha-2-Macroglobulin |
| | A2AP | alpha-2-antiplasmin |
| | AGT | Angiotensinogen |
| | APOA1 | Apoliporotein A1 |
| | APOC3 | Apoliporotein C3 |
| | ITIH1 | Inter-Alpha-Trypsin |
| | | Inhibitor Heavy Chain 1 |
| Anti-viral Histone proteins | H2A | Histone H2A |
| | H2B | Histone H2B |
| Complement proteins | C4b | Complement factor C4b |
| | C4BPA | Complement factor C4 |
| | | binding protein A |
| | CFBb | Complement factor C4 |
| | | binding protein B |
| Membrane attack complex (MAC) | C5a | Complement factor C5a |
| | C5b | Complement factor C5b |
| | C8G | Complement factor C8 |
| | C9 | Complement factor C9 |
| Soluble membrane attack | CLU | Clusterin |
| complex proteins (sMAC) | PON1 | Paraoxonase 1 |
| | VTN | Vitronectin |
| Platelet protein | RAP1AB | Ras-Related Protein Rap-1A |
| Immunoblobulins | IgG | |
| | IGHG1 | |
| | IGHG2 | |
| | IGHG3 | |
| | IGHA1 | |
| | IGHM | |
| | IGLC | |
| | IGLC | |

The inventors applied the multiplex assay to the same vaccine sample cohort characterised by Reynolds, C. J. et al. (Science 2021). These samples were then tested to see which immunocovidome proteins were affinity purified after incubation with three S1 spike proteins. These spike proteins were the spike protein from the Wuhan hul variant which is the spike protein most vaccines have been engineered against, the spike protein of the Kent variant (alpha B.1.1.7) which emerged in the UK in December 2020 and the spike protein of the South Africa variant (beta B.1.351) which emerged around the same time in South Africa (Reynolds, C. J. et al. Science 2021).

### Example 4—Comparison of Targeted Mass Spectrometry-Based IgG1 Assay Versus the Standard Roche Elecsys® Anti-SARS-CoV-2 ECLIA Spike

To determine how the MS assay compares with existing ELISA-based methods of detection of IgG, the S1 spike regions of SARS-CoV-2 from the Wuhan Hu-1, alpha B.1.1.7) and South African (B.1.351) strains were bound to a plate and used to screen the same samples previously analysed using the Roche Elecsys® anti-SARS-CoV-2 ECLIA spike. Using the assay described herein, each variant spike was immobilised on the plate, incubated with patient serum and the immunocovidome proteins analysed using LC-MS/MS. The LC-MS/MS assay was then used to analyse 3 groups of patients:
i) Vaccinated but no prior SARS-CoV-2 infection
ii) Vaccinated but have had a previous PCR-positive tests for SARS-CoV-2 infection
iii) Not vaccinated, no prior SARS-CoV-2 infection

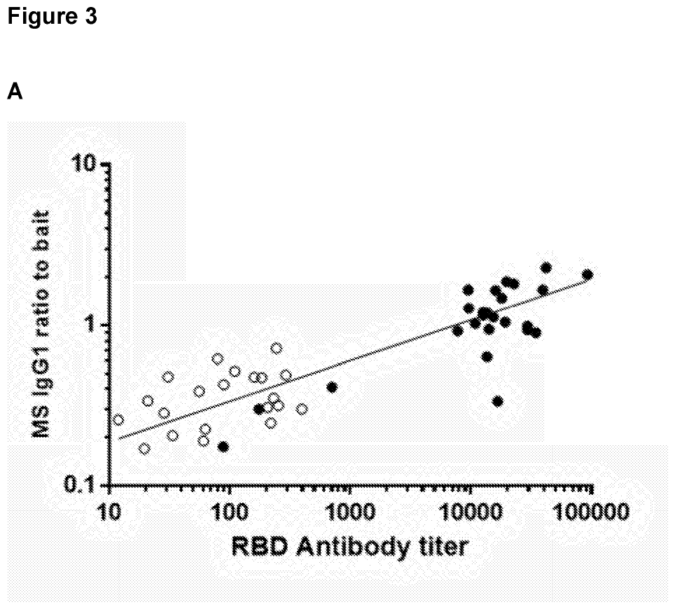

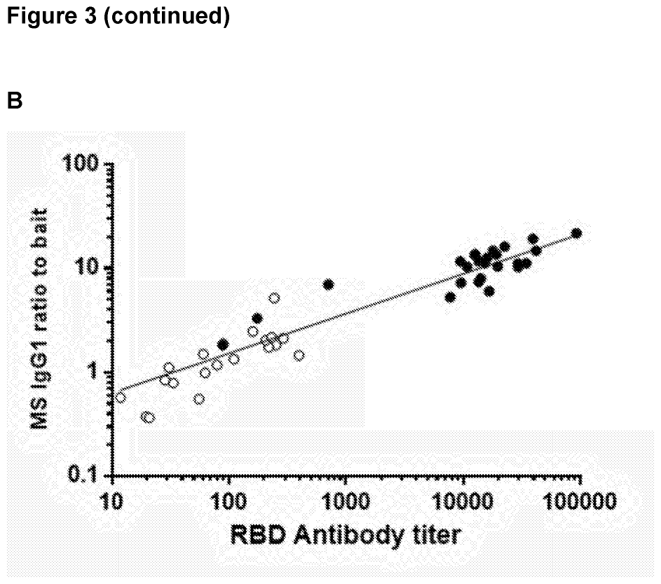

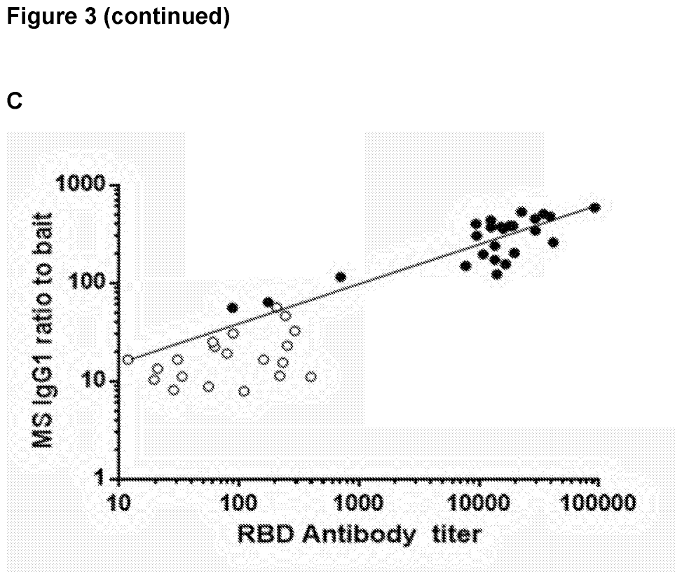

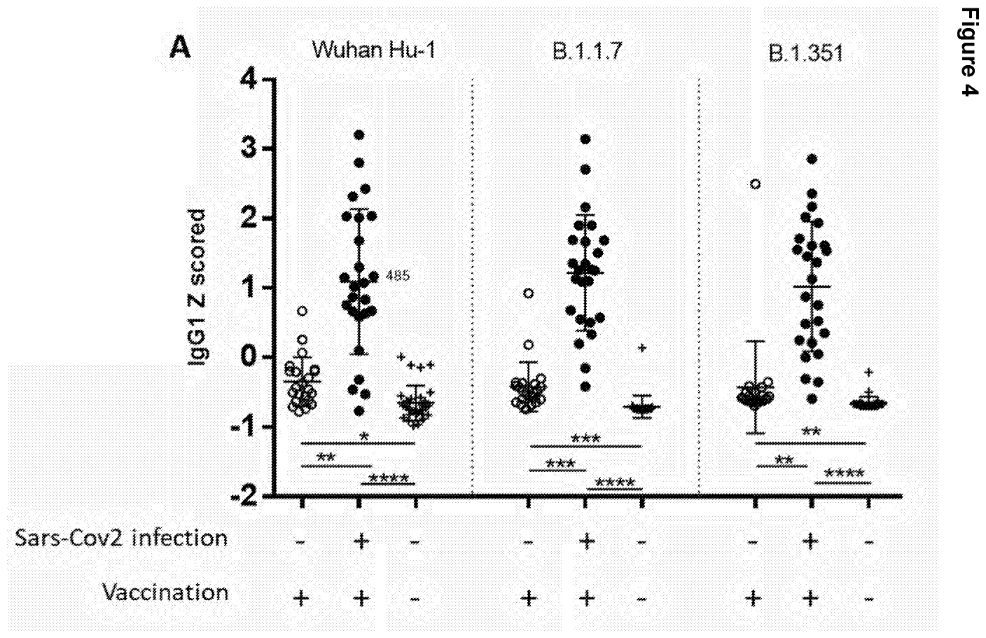

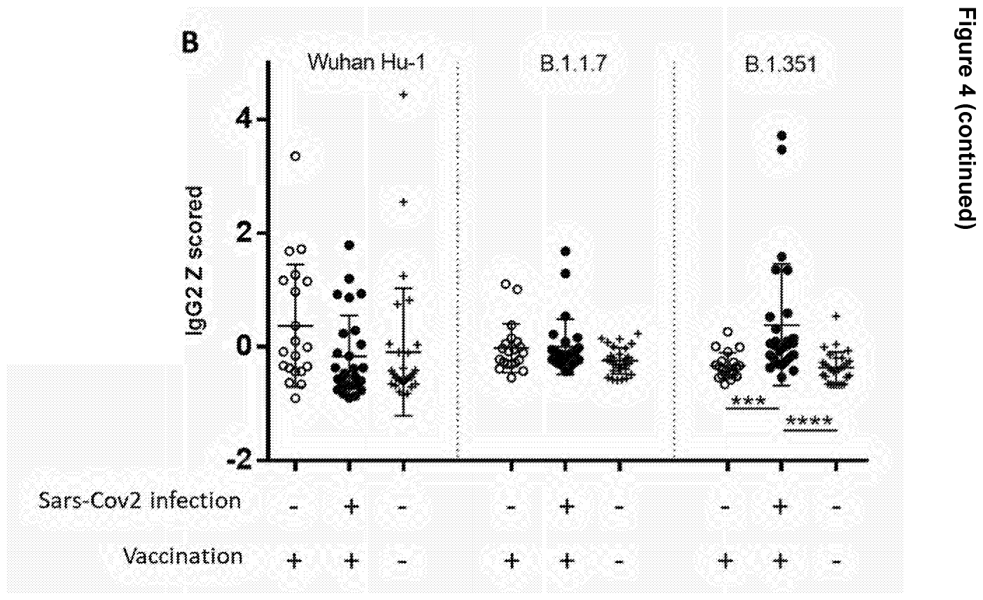

For the vaccinated groups the IgG1 levels analysed using the multiplex-LC MS/MS assay demonstrated a statistically significant correlation with the conventional Roche Elecsys® anti-SARS-CoV-2 ECLIA Spike assay. The results obtained for the Wuhan Hu-1, UK (alpha B.1.1.7) and South African (beta B.1.351) strains, were determined to be 0.798 (p<0.0001), 0.855 (p<0.0001) and 0.902 (p<0.0001), respectively (FIG. 3A-C). These results confirm a strong correlation between the technologies.

### Example 5—Comparison of Serum Immunoglobulin Response Against Different Variant Strains of SARS-CoV-2

### IgG1

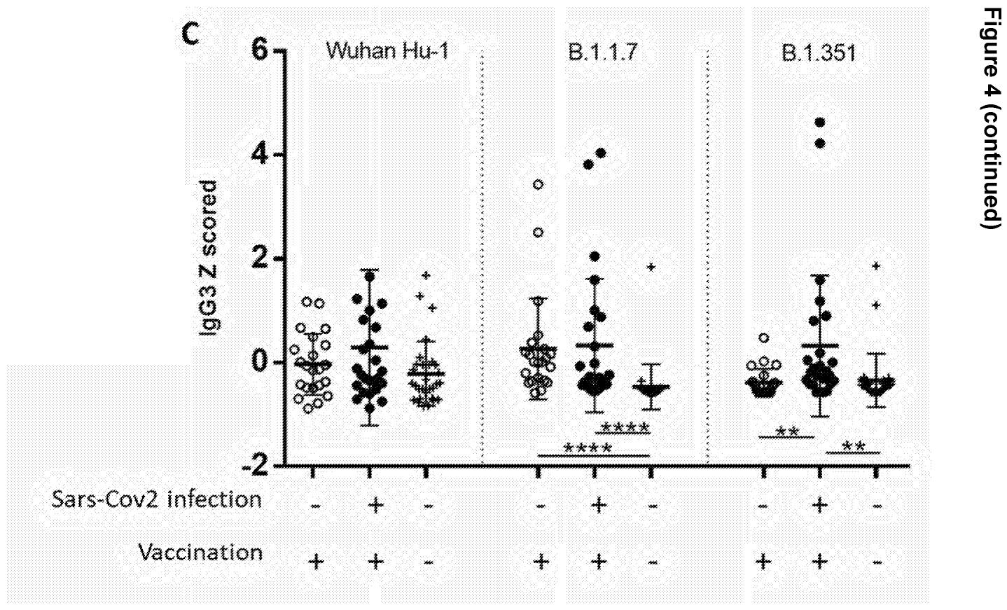

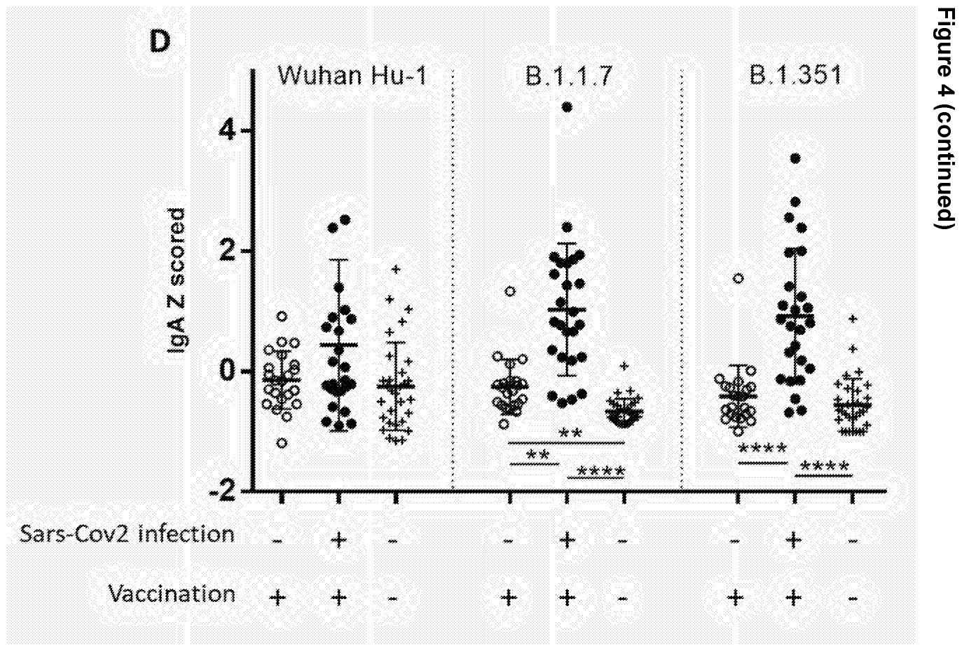

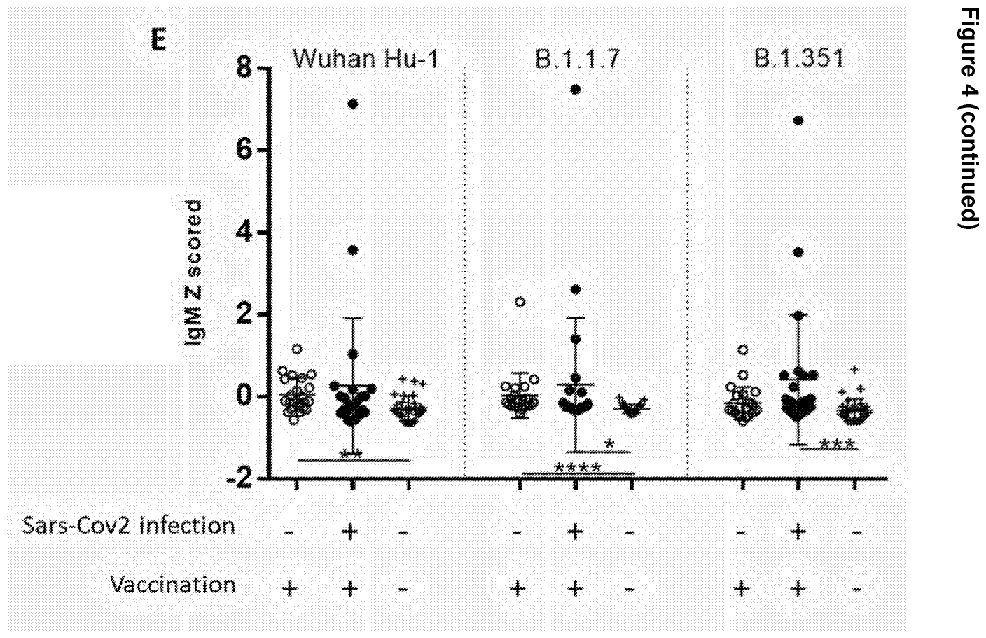

Multiple immunoglobulin classes and subtypes were measured in the vaccinated cohort. Levels of IgG1 (which is the main immunoglobulin G subtype) were significantly increased in all vaccinated individuals compared to those who had no infection or vaccine across all strains (FIG. 4A). In accordance with previous findings, the largest fold change elevation of IgG1 levels was observed in the prior-infected/vaccinated group relative to the unexposed group. The Wuhan Hu-1, alpha and beta variants demonstrated a 4.9, 195 and 69 average fold elevation in IgG1 levels, respectively. However, the fold-change increases in the IgG1 levels between the unexposed/unvaccinated group compared to the vaccinated group with no previous infection, although statistically and significantly elevated, were only 1.67-, 32 and 4.57 fold elevated compared to the Wuhan hu-1, alpha and beta variants, respectively.

### IgG2

IgG2 plays a role in protection against protein antigens but is predominantly responsible for anticarbohydrate IgG responses. No changes were observed in IgG2 serum levels specific to the Wuhan-hu1 or alpha B.1.1.7 strains (FIG. 4B). However, it was observed that the vaccinated and previously-infected group had a significantly increased IgG2 response compared with vaccinated naïve and unexposed controls which appeared to be driven by a subset of participants (FIG. 4B).

### IgG3

IgG3 is a lower abundant subtype of IgG but is expressed earlier in response to infection² (Ferrante, A. et al. Virology 2004). IgG3 levels did not change in response to the Wuhan-hu1 S1 protein but the alpha B.1.1.7 strain elicited a response in vaccinated individuals irrespective of a previous infection. The response to the beta B.1.351 variant was similar to that observed for IgG2 with only an increase in the previous infection group largely driven by a subgroup of individuals (FIG. 4C).

### IgA

The other immunoglobulin classes A and M were also included in the assay. IgA is also abundantly present in human serum. However, the biological relevance of IgA and the regulation of its effector functions are still poorly understood. The inventors observed no change in IgA response against the Wuhan-hu1 strain in the vaccinated cohort. However, a similar profile for that elicited by IgG1 for the IgA response to the alpha (B.1.1.7) strain with a moderate but significant increase in vaccinated infection-naïve individuals and a greater response in the previously-infected group (FIG. 4D) was observed. The IgA response to the beta (B.1.351) strain showed a similar pattern as observed for IgG2 and IgG3 with no change in the vaccine-naïve group. Only a significant increase in the levels of each IgG in the previously-infected group was seen.

### IgM

IgM is typically the acute response immunoglobulin. Levels of IgM will increase shortly after infection and decrease before IgG becomes the dominant immunoglobulin. A small significant increase for the vaccinated infection-naive group against the Wuhan-hu1 strain was seen. No change was observed for the vaccinated and previously-infected group. A more significant 2.91 fold increase in the vaccinated infection-naïve group against the alpha B.1.1.7 variant was seen. A slight significant increase in the previously-infected group was also seen. In this group, a small number of samples were dominated by large IgM values. IgM is only increased significantly in the vaccinated previously-infected group against the beta variant (B.1.351). Unlike the Wuhan-hu1 and alpha (B.1.1.7) strains there was no change in the vaccinated infection-naïve group (FIG. 4E).

### Example 6—Comparison of the Targeted Mass Spectrometry-Based IgG1 Assay with Neutralising Antibody Levels

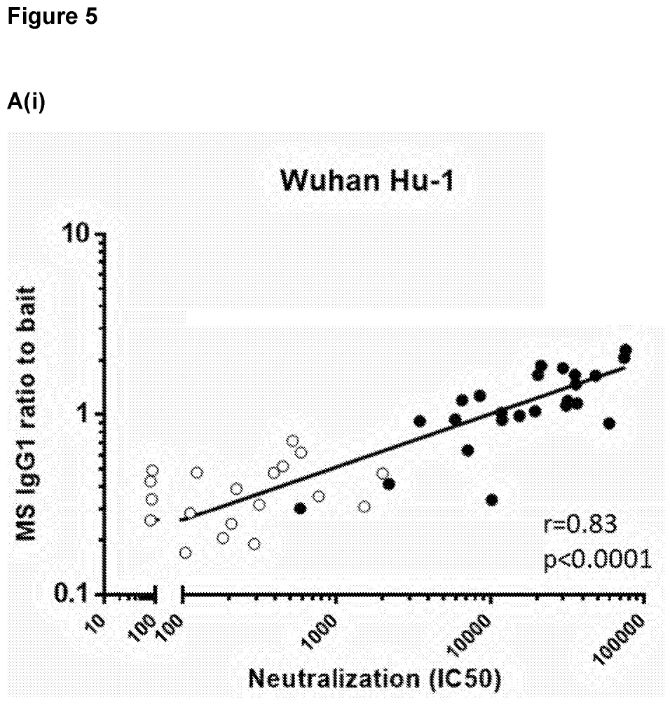

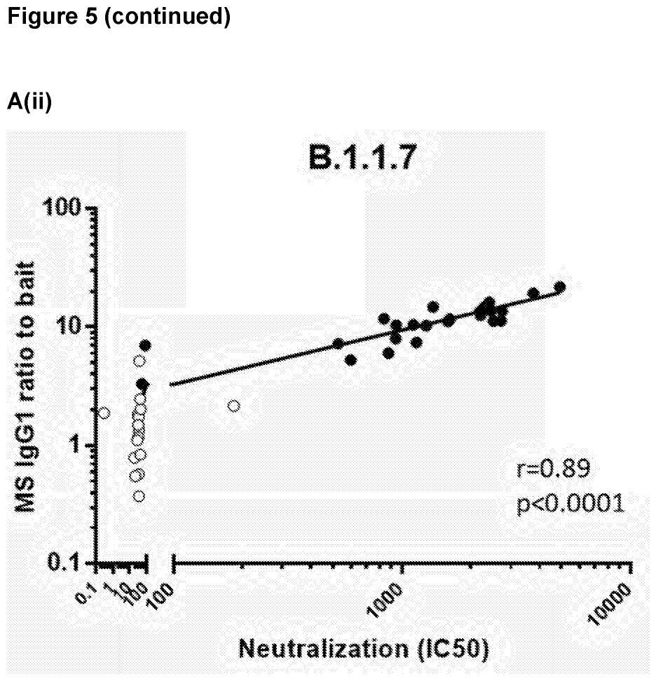

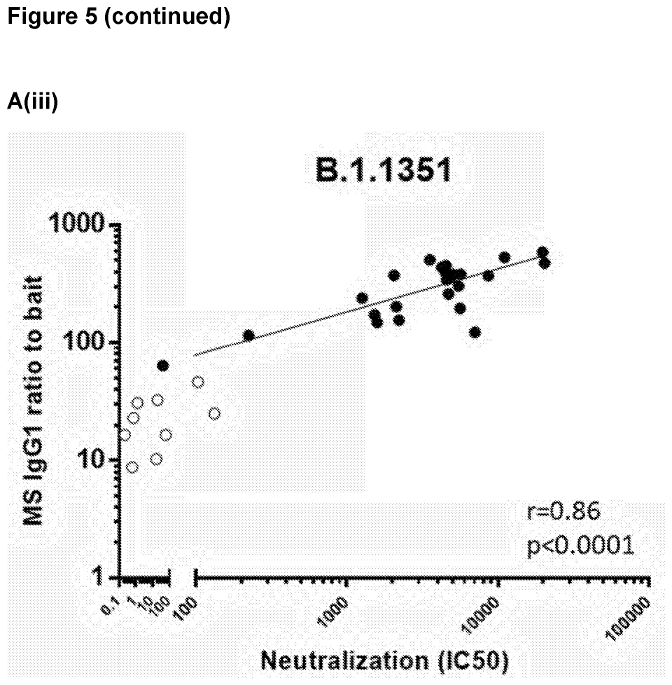

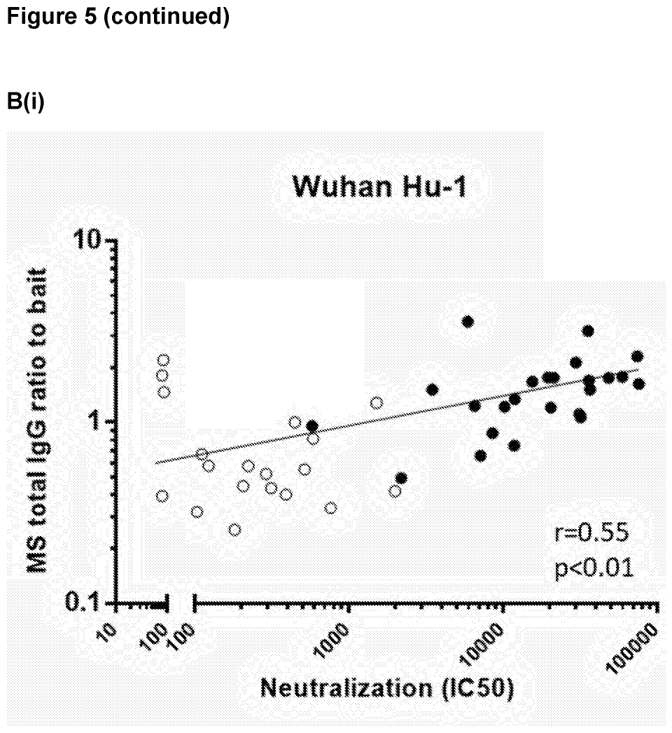

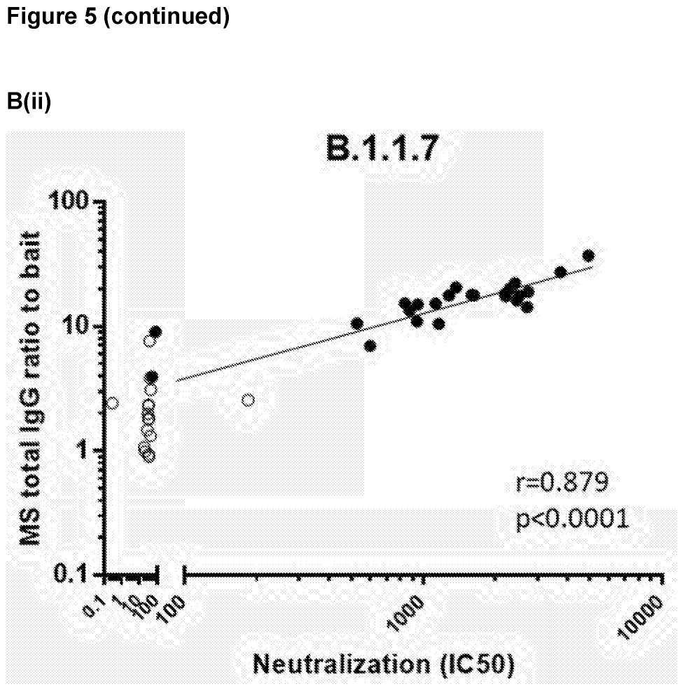

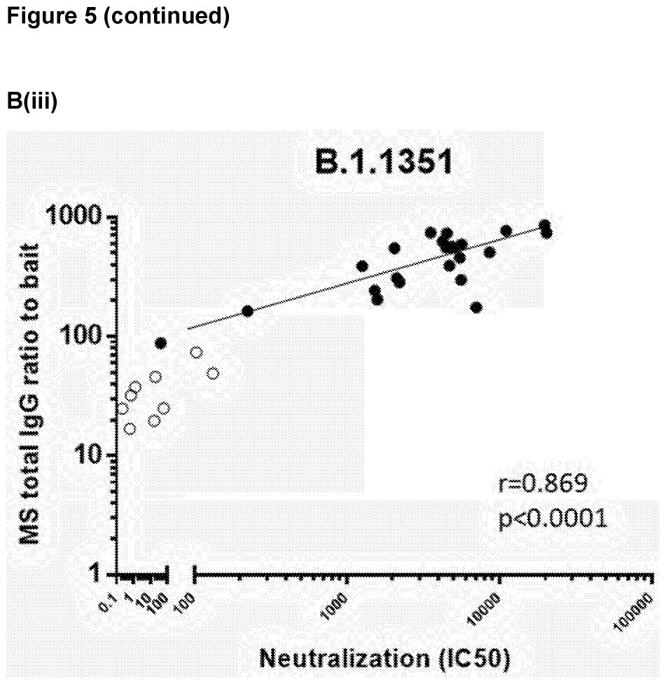

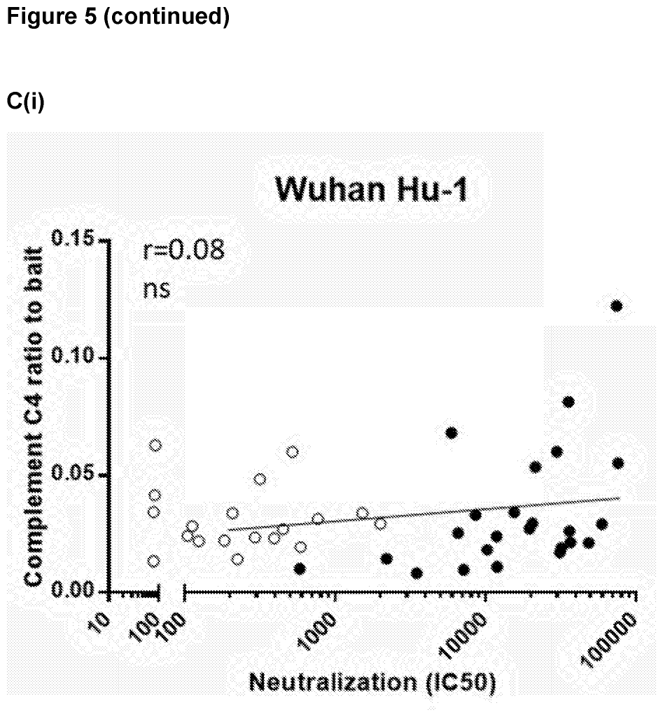

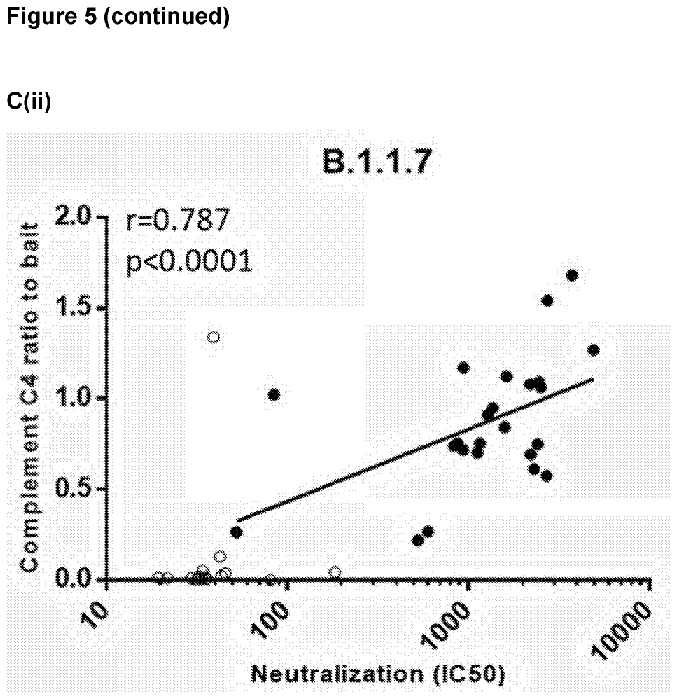

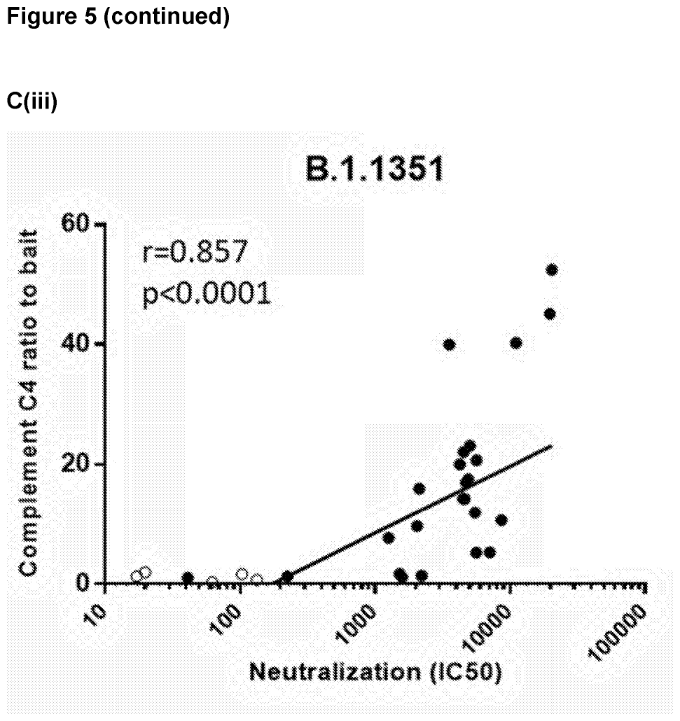

To determine if the multiplex-LC MS/MS based assay could also identify neutralisalising antibody levels, we compared neutralising antibody results from Reynolds et al. (Reynolds, C. J. et al. Science 2021) with the results obtained from the multiplexed bait assay (FIG. 5A). A strong positive correlation was observed for all strains with the conventional neutralising antibody assay (r>0.83) indicating that multiplex-LC MS/MS-based results could also determine levels of neutralising antibodies. The results demonstrated a slightly improved correlation for the Roche Elecsys® RBD antibody titre values (r=0.89) compared with those obtained with multiplex-LC MS/MS (r=0.83) against the Wuhan Hu-1 strain. However, correlation coefficients were slightly better for the LC-MS/MS IgG1 assay than the Roche Elecsys® RBD antibody titre values against the alpha (B.1.17) (r=0.89 versus 0.87) and beta (beta B.1.351) (r=0.86 versus 0.84). All data indicated a strong correlation between neutralising antibody levels and IgG1 data obtained using the multiplex-LC MS/MS based assay. Furthermore, as the LC-MS/MS assay measures multiple IgG subtypes and immunobased proteins, a comparison between neutralising antibodies and total IgG (1-4) was performed (FIG. 5B). Total IgG correlated well for both the alpha (B.1.1.7) and beta (B.1.351) strains as IgG (r>0.869), but a weaker correlation was observed for the Wuhan hu-1 strain (r=0.55 for total IgG vs r=0.83 for IgG1). This data indicates that other IgG subclasses have less specificity for neutralising antibodies against the Wuhan-hu1 strain (FIG. 5B). The other protein detected in the immunocomplex and which also demonstrated a significant correlation with neutralising antibodies, was found to be complement C4 (FIG. 5D). No correlation of complement C4 levels to neutralising antibodies was observed with the Wuhan-hu1 strain. However, a statistically significant correlation of r=0.787 was shown for the alpha (B.1.1.7) variant and an even stronger correlation r=0.857 was observed for the beta (B.1.351) variant, (FIG. 5C).

### Example 7—Comparison of Serum Immunoglobulin Response with Cellular Immune Response Data

Analysis of B cell and T cell responses as measure by ELISPOT assays for the vaccine cohort was also reported by Reynolds et al. (Reynolds, C. J. et al. Science 2021). Cellular response data was only observed in the previously-infected and vaccinated cohort. Therefore the multiplex LC-MS/MS data was compared only with this group. FIG. 6 shows a correlation matrix for the significant comparisons. A weak correlation (r=0.36) was observed with IgG1 and S1 specific IgG antibody secreting cells but no correlation was observed with the other antibody classes. The T cell responses against the spike protein and mapped epitope peptides (MEP) however appeared to show a significant correlation (r=0.45-0.55) with IgG3 and IgA but not with IgG1.

### Example 8—Complement Response to S1 Variants

Through proteomic profiling of the S1 bait capture the inventors identified other proteins associated with the immunocomplex and included them in the multiplex assay. These included complement C4 and C4 binding protein and proteins of the membrane attack complex complement C5, C8 and C9. The complement system is an important component of the innate immune response to virus infection for which immunoglobulins activate complement in response to infection and complement will contribute to neutralisation³ (Blue, C. E. et al. Virology 2004). LC-MS/MS complement analysis of the vaccine cohort revealed that complement C4 correlated with neutralising antibodies (nAbs) and when compared across vaccine groups a significant increase in the vaccinated groups for all strains was observed (FIG. 7). The vaccinated infection-naïve group showed a significant 1.4 fold increase against the Wuhan Hu-1 S1 and a weakly significant 9.36 fold increase against the alpha (B.1.1.7) variant and no significant increase against the beta (B.1.351) variant. The vaccinated and previously-infected group however showed a significant increase for all strains. The response to the Wuhan-hu 1 in the previously-infected group was comparable to the vaccinated infection-naïve group with a greater 4.2 fold change. For the alpha (B.1.1.7)S1 a large average 91 fold increase which is significantly elevated compared to the vaccine naïve group as well as the unexposed group was observed. For the beta (B.1.351) variant, a 38 fold increase in the vaccine previously-infected group which also is significantly increased compared to both vaccine infection-naïve and unexposed groups was observed. This analysis indicated that the first dose of a vaccine against SARS-CoV-2 is able to elicit a complement response against Wuhan-1 and alpha (B.1.1.7) strain/variant but not the beta (B.1.351) variant. A second exposure to SARS-CoV-2 is required to elicit a response. Table 8 shows a summarised heat map of the average fold changes for the other complement proteins which show those complement factors, C5-C9, which are associated with the membrane attack complex are not changed in response to the Wuhan-h1 strain but are increased in the previously-infected group against the variant strains, alpha and beta.

### Example 9—Applicability of Targeted Mass Spectrometry-Based Assay to Dried Blood Spots and Saliva Spots/Lollipops

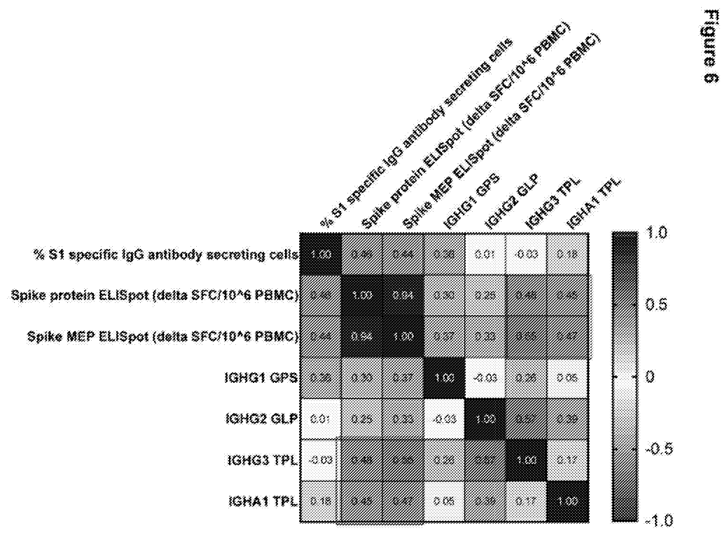

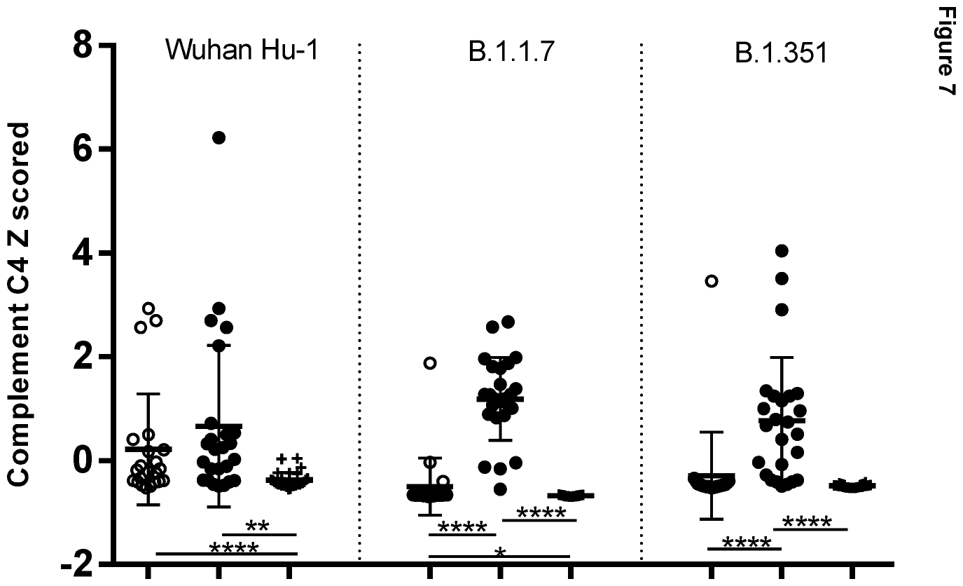

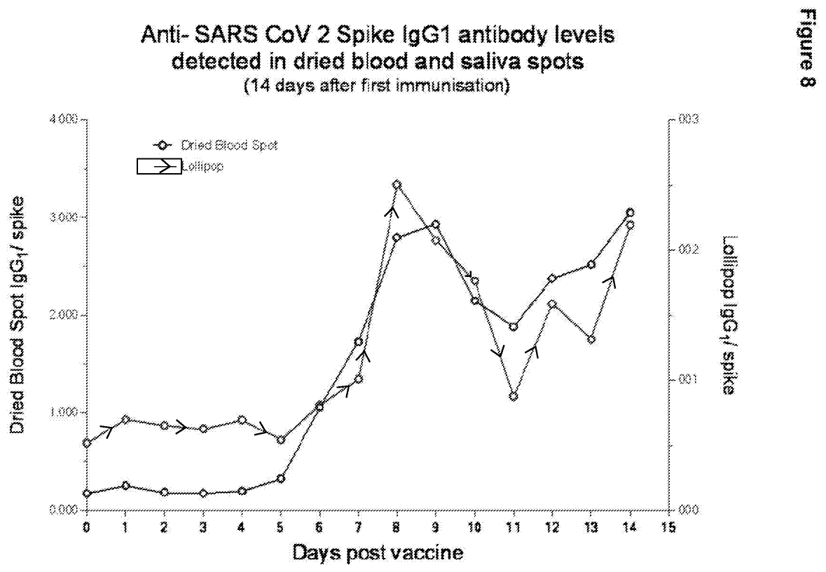

The assay described herein (S1 Wuhan hu-1) was adapted for analysis of dried blood spots and saliva spots. Blood and saliva samples were taken from a single participant who had a previous SARS-CoV-2 infection in March 2020 using a lollipop. These samples were collected after a first vaccine dose (Astra Zeneca vaccine) and sampled at the same time. FIG. 8 shows the levels of IgG1 up to 14 days post infection. Good correlation between DBS and saliva spot (r>0.9) with IgG1 levels beginning to increase after day 5 was observed. This pattern is characteristic of an IgG response after antigen exposure.

### Example 10—Discussion

There is a need for improved, faster and more easily available testing of the immune response to SARS-CoV-2 infection for large scale monitoring of a vaccine response. Currently antibody-based testing either monitors IgG or just measures the total binding affinity to a bait antigen. Whilst these tests are accurate and convenient they are not specific and therefore provide limited information about the immune response. This information is becoming necessary to acquire with the emergence of SARS-CoV-2 variants and the effects of current vaccines against them. Currently to obtain this information measurement of multiple immunoglobulins IgG, IgA and IgM and the associated complement proteins require separate ELISA based assays. The multiplex SARS-CoV-2 immunocomplex assay developed by the inventors can simultaneously measure multiple molecules including proteins that are associated with the immune response to SARS-CoV-2 in one analysis, thereby reducing cost and the amount of time compared to conventional assays. The methods described herein also provide absolute accurate quantitation over a linear range (20-2500 fmol) without dilution of samples which is required in conventional assays. The levels of IgG1 obtained by the methods described herein are comparable to those obtainable by the Roche Elecsys® Anti-SARS-CoV-2 assay.

The inventors included a peptide to measure total IgG which was shared between all four IgG subtypes. Comparing total IgG specificity with IgG1 they observed that IgG1 is the main IgG associated with neutralising antibodies (nAbs) to S1 protein particularly against the original Wuhan-hu-1 S1 strain. Correlation with neutralising antibody (nAb) data show good correlation with improved r values compared to ELISA data for the alpha (B.1.1.7) and beta (B.1.351) S1 assays.

When comparing LC-MS/MS multiplex data within S1 assays the results were standardised for comparison. IgG1, the main IgG subtype showed significant changes for all groups for all strains with a moderate response for vaccinated infection naïve individuals and a large degree of change in HCW who had prior infection, corresponding to the pattern of response previously described¹ (Reynolds, C. J. et al. Science 2021). Other IgG subtype responses were not observed against the Wuhan hu-1 S1. However changes were observed against the variants. Interestingly vaccine status appears not to elicit an IgG3 response against the beta B.1.351 S1 but did for the alpha B.1.1.7. Only the group who had a previous infection showed an IgG3 response to the beta B.1.352 strain. IgG3 comprises only a minor proportion (5-8%) of total IgG. However, its importance in the control and/or protection from a range of pathogens, including viruses is only recently being highlighted⁴ (Damelang, T. et al. Trends Immunol 2019). IgG3 has a number of antigen-specific effector functions such as immune cell-activation, neutralization, and activation of complement pathways (Lee, C. H. et al. Nat Immunol 2017), (Vidarsson, G. et al. Frontiers in immunology 2014). Due to its superior affinity to FcγR it is the most functional subclass, closely followed by IgG1 (Ferrante, A., et al. (Pediatr Infect Dis J 1990, 9 (8 Suppl), S16-24.).

The interaction of IgG3 with FcγRs on effector cells such as neutrophils, monocytes, macrophages, or natural killer (NK) has been increasingly recognized as a critical immune response against a range of infections from viruses, bacteria, and parasitic pathogens⁴ and the inventors observed in their comparison with T-cell response data to spike protein and spike mapped epitope peptides a relationship with IgG3 and IgA this corresponds with previous observations that IgG3 is better at interaction with effector cells. Recent HIV vaccine studies suggest that despite being of low titers, IgG3 is a potent effector of vaccine-induced humoral immune responses as observed for the RV144 vaccine trial, the only human HIV-1 vaccine trial to achieve partial efficacy, high IgG3 binding to HIV-1 envelope protein correlated with a lower risk of HIV-1 infection (i.e., increased vaccine efficacy) relative to controls (Yates, N. L. et al Science translational medicine 2014). The IgG3 data for the vaccine cohort the inventors have studied indicates there may be less IgG3 mediated vaccine efficacy for the beta B.1.351 strain at first dose/exposure. No change is observed against the wild type wuhan h1 strain which the vaccines are targeted towards as the IgG3 response for this strain may be more acute/faster and the response has already declined at 22 days post vaccine. This is plausible as IgG3 has a shorter half-life (Stapleton, N. M. et al. Nat Commun 2011). This indicates that kinetics of IgG3 response should be monitored against strains and vaccines.

In SARS-CoV-2 infection seroconversion for IgA has been shown to be sooner at 2 days after onset of symptoms than IgM and IgG at 5 days⁹ (Yu, H. Q. et al. Eur Respir J 2020). Sterlin et al. report that specific humoral responses are dominated by IgA antibodies and IgA contributes to virus neutralization to a greater extent compared with IgG (Sterlin, D. et al. Science translational medicine 2021). Influenza-specific IgA has been shown to be more effective in preventing infections in mice and humans compared with influenza-specific IgG, and elevated IgA serum levels have been correlated with influenza vaccine efficacy (Liew, F. Y. et al. Eur J Immunol 1984), (Asahi-Ozaki, Y. et al. J Med Virol 2004). IgA levels are not affected against the Wuhan H1 S1 protein. However IgA response to the alpha (B.1.1.7) variant is increased in vaccinated individuals with greater response in those who had previous infection. However like with IgG3 only an increase in the pre-infected vaccinated group was seen against the beta (B.1.351) variant also indicating a reduced immune response to beta (B.1.351) in those who've had only one dose of vaccine. As with IgG3, IgA was observed to have a stronger relationship with cellular response data against spike protein and spike MEP than IgG1.

IgG3 and IgA may well act as surrogate cell response markers. An IgM response is often observed first before IgG and declines as IgG takes over. A significantly small increase in IgM against the Wuhan H1 and alpha (B.1.1.7) strains was observed in the vaccinated group without prior infection and less so in those who had a previous infection. However this is not observed against the beta (B.1.351) variant which fails to elicit a response in the vaccinated infection naïve group. Only the pre-infected vaccinated group show a small significant change. This corresponds with other Ig observations in a lack of immune response to a first exposure against the beta (B.1.351) variant.

Proteomic analysis of the bait capture identified other proteins associated with the immune response to the S1 protein in seropositive individuals. Of these were complement proteins including those involved with the membrane attack complex C5-C9. Targeted LC-MS/MS assay of complement C4 showed the greatest change in the vaccine cohort. C4 was increased in both vaccine groups and pre-infection status did not appear to have an effect. However this is not the case with the response to the variants.

A small response is observed against alpha (B.1.1.7) and no response is observed against the beta (B.1.351) in the vaccine infection naive group. A large response in C4 is seen against both variants in the pre-infection vaccinated group similar to what is observed for other immunoglobulins. Complement is activated by immunoglobulins against viral infection and is required for neutralisation. Correlation analysis of C4 with neutralising antibody data showed a significant correlation only against the variant S1 and not the wild type Wuhan H1 S1. These observations could be due to contribution of non-neutralising antibodies.

Neutralizing antibodies are an important specific defence against viral infection. A neutralizing antibody might block interactions with the receptor, or might bind to a viral capsid in a manner that inhibits uncoating of the genome. Only a small subset of the many antibodies that bind a virus are capable of neutralization. After an infection, it can take some time for the host to produce highly effective neutralizing antibodies but these persist to protect against future encounters with the virus. Non neutralising (natural) antibodies will also be present at infection and will bind to the virus but not in a manner to prevent infection. In a study investigating the mechanism of non-neutralising antibody action on parainfluenza virus type 3 it was observed that non neutralising antibodies have an absolute requirement for the classical pathway of complement activation and lytic terminal complement components, and viral lysis to neutralise the virus (Vasantha, S. et al. J Virol 2007). This mechanism of virus-binding, non-neutralizing natural antibody is also described by Jayasekera et al. (J Virol 2007, 81 (7), 3487-94). Neutralising antibodies however act by binding to a virus, in a manner that blocks infection and are not reliant on complement.

This may explain why there is less complement bound to the wuhan S1 as there are more neutralising antibodies bound that therefore do not require complement. This indicates the antibody response to the variant strains is due to presence of more non neutralising antibodies that do require complement. Thus simultaneous measurement of complement may provide an indirect way of measuring neutralising and non-neutralising antibodies against the S1 proteins.

### Example 11—Conclusion

The inventors have developed an LC-MS/MS method to accurately monitor the immunocomplex against SARS-CoV-2 S1 spike protein in patient samples. The method compares well with other accepted methods but adds much further information about the immune response and can be used to monitor neutralising and non-neutralising antibody activity.

### Example 12—Abstract

Determining the protection an individual has to SARS-CoV-2 variants of concern (VoC) will be crucial for future immune surveillance, vaccine development and understanding the changing immune response. As further variants emerge, current serology tests are becoming less effective in reflecting neutralising capability of the immune system. The antigen-antibody response consists of an immunocomplex of not just antibodies but other proteins, particularly the complement system whose involvement and contribution to the antigen response is poorly understood. To address this we devised a more sophisticated and informative assay to current ELISA based serology using a multiplexed, baited, targeted-proteomics test for direct detection of multiple proteins in the SARS-CoV-2 anti-spike antibody immunocomplex.

Using this approach, we have characterised and identified specific responses to Wuhan Hu-1, Alpha (B.1.1.7), Beta (B.1.351), Delta (B.1.617.1) and Omicron (B.1.1529) SARS-CoV-2 variants. Analysis of serum from individuals collected after infection, or first and second dose vaccination demonstrate that this approach shows concordance with existing serology and neutralisation tests. Our assays show altered responses of both immunoglobulins and complement to the Alpha, Beta and Delta VoC and a reduced response to the Omicron VoC. We were able to identify vaccinated individuals who had a prior infection, by showing they had a greater IgA response against all VoC (p>0.01) and also greater complement C4 and C9 binding against the variants. We observe that C1q is closely associated with IgG1 levels (r>0.82) but the ratio of IgG1 to C1q changes against the Delta and Omicron VoC with C1q better reflecting the reduction of neutralisation to VoC than IgG1. Our data indicates that looking at additional immunoproteins beyond IgG, provides important information about our understanding of the response to infection and vaccination. This will be important as new variants of concern emerge and has the potential to benefit public health immunosurveillance and particularly new vaccine design or trials.

### Example 13—Introduction

After the first cases of SARS-CoV-2 were identified in late 2019, 2020-2021 saw the development and roll out of the world's fastest and largest global vaccination programs. However, with potential waning immunity over time (Gaebler et al., 2021) and the impact of infection from emerging Variants of Concern (VoCs) (Reynolds et al., 2021a), it is apparent that there is a need for better and more informative testing (Abbasi, 2021). This will help determine the clinical need for booster vaccination and timing of the boost itself. First generation tests were rolled out at scale but are largely based on simple non-specific binding to the prototypic Wuhan-Hu-1 spike sequence. It is now clear that these methods overestimate the actual protective immunity against VoC (Reynolds et al., 2021a, b and 2022). Current technologies that directly measure accepted correlates of protection such as neutralising antibodies (nAbs) scale poorly for clinical utility, while serological approaches (ELISA or ECLIA), measure only part of the antibody response and omit measurement of effector Fc antibody functions such as complement involvement (Ju et al., 2020; Nie et al., 2020; Yu et al., 2020).

To aid in understanding the antibody response to SARS-CoV2 we have developed a methodology that includes a ‘bait and capture’ system, followed by a multiplexed and targeted proteomic liquid chromatography tandem mass spectrometry (LC-MS/MS) analyses. The combination of immunocapture with the multiplexing capability to look at multiple proteins involved in the immune response and high accuracy of mass spectrometry quantitation, makes this an extremely powerful and more informative combination. In addition, tandem mass spectrometers are also routinely used for small molecule clinical assays in most UK pathology laboratories and are therefore platforms which could be utilised for targeted proteomic assays. It's only recently, with improving technology that they are becoming recognised for their potential clinical application for multiplex protein analysis (Smit et al., 2021).

In this work we describe how we have used this novel assay to compare with previously determined immune correlates (Reynolds et al., 2021a; Reynolds et al., 2021b; Reynolds et al., 2022) in serial samples, in response to vaccination, infection and an individual's potential protection against VoC. This analysis was performed using serum samples from the COVIDsortium study (Manisty et al., 2021a; Manisty et al., 2021b; Reynolds et al., 2021b; Reynolds et al., 2022; Reynolds et al., 2020; Treibel et al., 2020) where previously detailed longitudinal immunological analysis had been carried out. This unique cohort included healthcare workers (HCW) with and without laboratory confirmed SARS-CoV-2 infection, during the first UK wave with the Wuhan Hu-1 strain and after one and two dose vaccination (Pfizer/BioNTech BNT162b2) (Manisty et al., 2021a; Reynolds et al., 2021b; Reynolds et al., 2020; Reynolds et al, 2021a). Our analyses demonstrates that the conventional measurement of IgG1 is insufficient to determine an individuals complete immuno-response or protection due to infection and vaccination. We show that responses to infection and vaccination can be heterogeneous from person to person and by broadening the portfolio of those biomarkers involved in the monitoring of the immune response, this assay provides a more informative picture of immune responses. This assay could be used to estimate an individual's immune potency against VoC but also aid in design of future vaccine trials.

### Example 14—Results

### The Development of a Multiplex LC-MS/MS Assay for Measuring Antibody Mediated Response to SARS-CoV-2 Spike Antigen

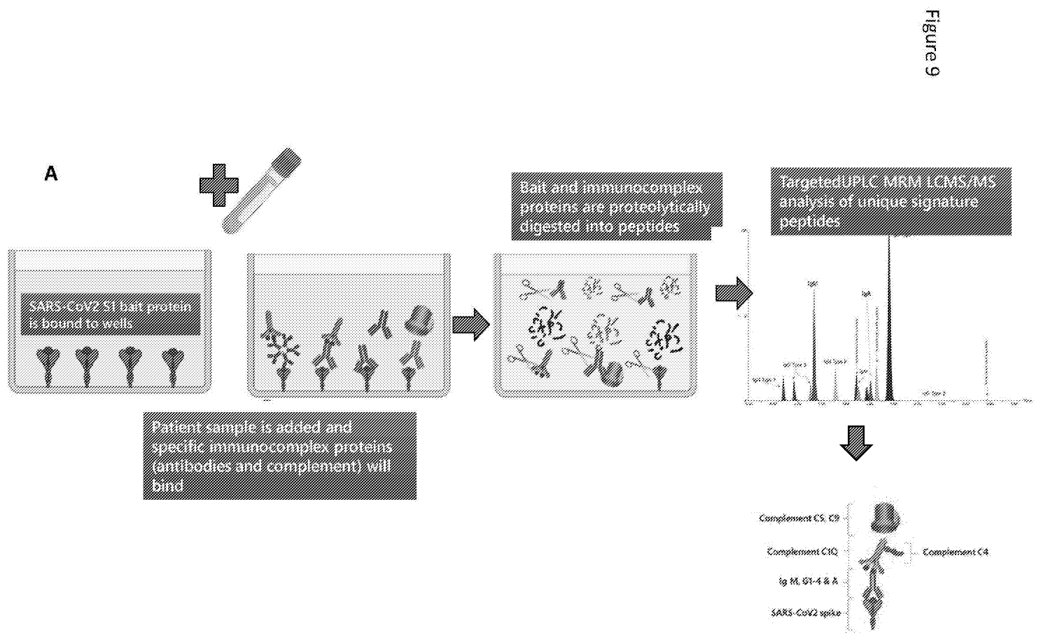

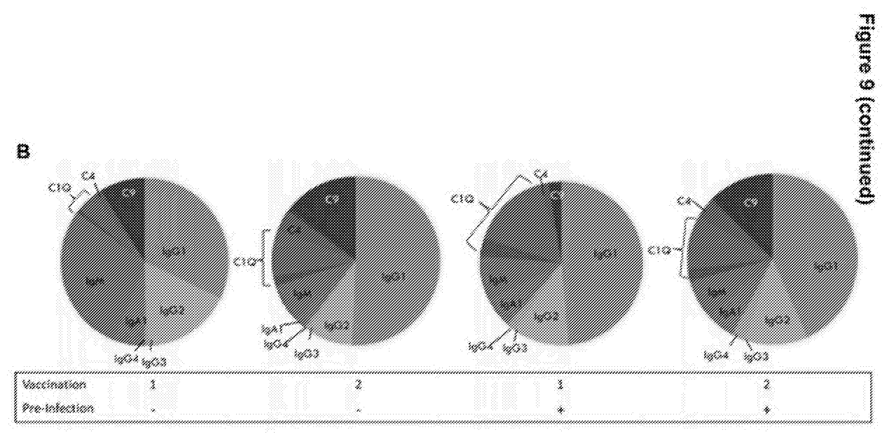

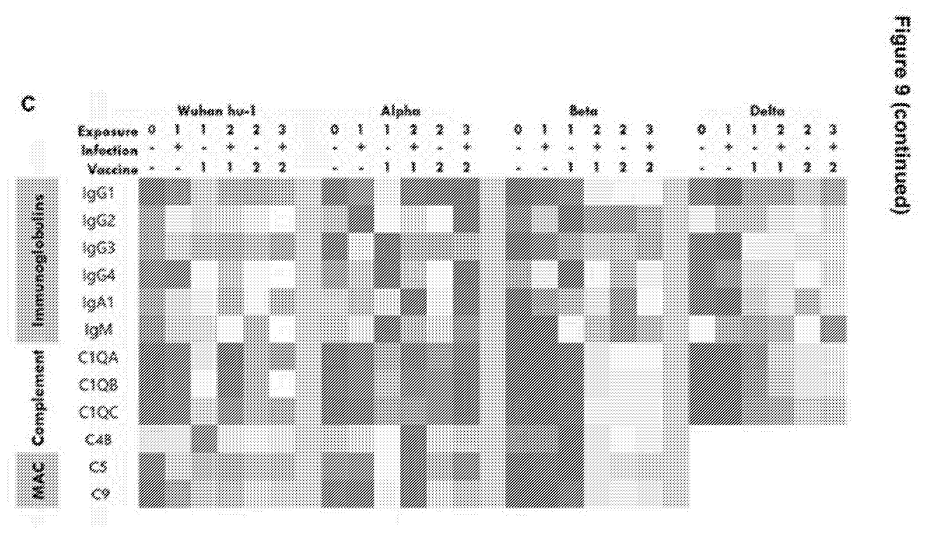

The multiplex assay developed is significantly more sophisticated and informative because of its ability to quantitate simultaneously and accurately, all major antibody species and their subclasses, as well as key components of the downstream complement pathway. The rationale behind inclusion of complement proteins is due to their involvement in formation of antigen-antibody complexes. FIG. 9A is a schematic representation of how the assay captures and analyses a patient serum immunocomplex specific to the SARS-CoV-2 spike region. A recombinant SARS-CoV-2 S1 spike protein from any VoC is first bound to a 96-well plate in a simple procedure described below. Patient serum is incubated with the bait for 60 min to capture the immunocomplex and non-specific proteins washed away. All immunocaptured proteins including the spike, are trypsin digested and the unique signature peptides analysed by a targeted LC-MS/MS analyses. The assay was capable of identifying and quantitating the immunoglobulins IgG1, 2, 3 & 4, IgA1, IgM and the complement factors C1q, C4b, and C9 using 10 ml of serum with a CV range of 1.7%-13.6% for high response QCs and 1%-15.3% for low response QCs. To improve reproducibility over existing immunodetection assays, results were expressed as a ratio of each immunological protein to the SARS-CoV-2 spike (using a S1 peptide common to all variants). This improves the CV % by a factor of ˜10-fold i.e. a standalone value CV for IgG1 is reduced from 13.8% to 3.4% if the value is ratioed to spike bait. Internal monitoring of bait binding also provides a quality assurance for plate preparation.

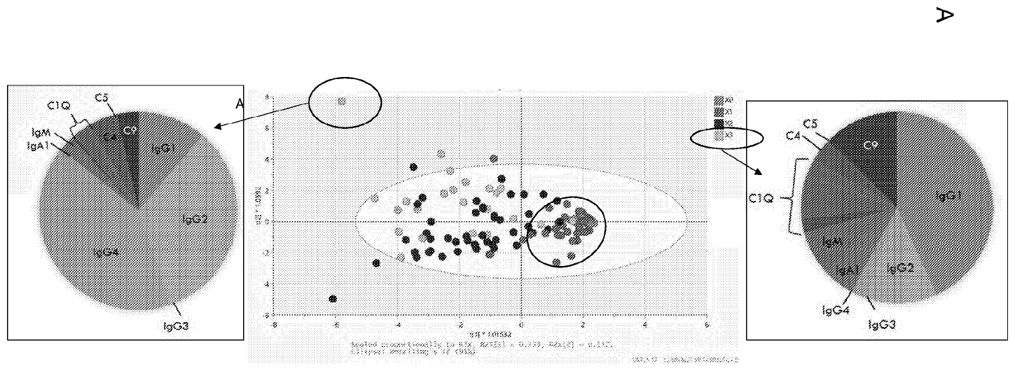

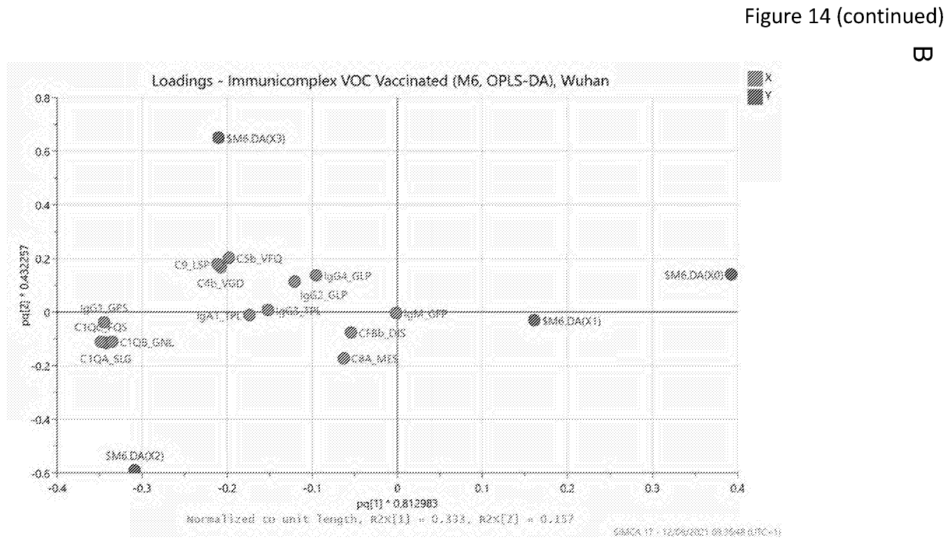

FIG. 9B shows how the profile of the immunocomplex changes according to vaccination and prior infection. A heatmap summary of the proteins included in the immunocomplex and their response to exposure and variants is given in FIG. 9C and summarises the increase of the immunocomplex with increasing exposure against all SARS-CoV-2 variants. However, a notable reduction of detectable immune activity against the Beta VoC can be observed compared to other variants analysed. A strength of our assay is to measure all major antibody types simultaneously, allowing comparison of inter-individual and exposure isotype heterogeneity. When all components of the immunocomplex are viewed collectively (supplementary FIG. 14) a wide variation in those with 2 or more antigen exposures can be observed with clear outliers. Closer inspection of the outliers highlights that these individuals have an atypical immunocomplex profile with one interesting individual who has a dominant IgG4 response. IgG4 is associated with anti-inflammatory properties as it can undergo fab-arm exchange thereby limiting effector functions (van der Neut Kolfschoten et al., 2007). This indicates that a ‘one size fits all’ strategy using the current Elecsys assays does not provide us with enough information if we are to determine and study an individual's current immune status.

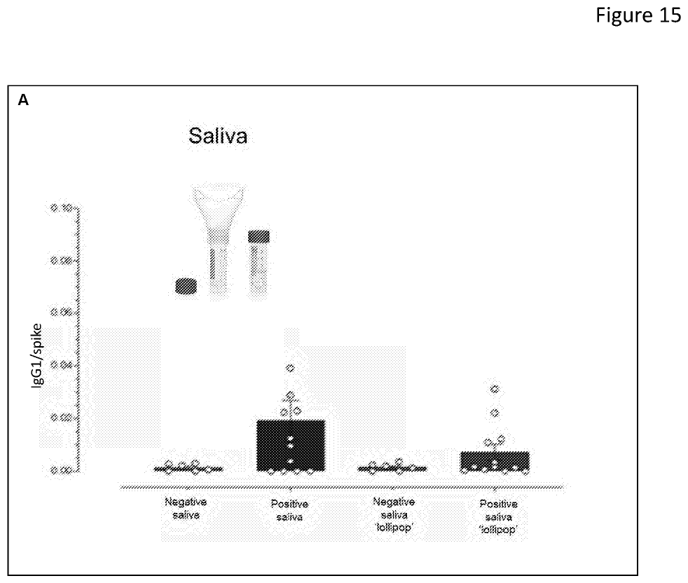

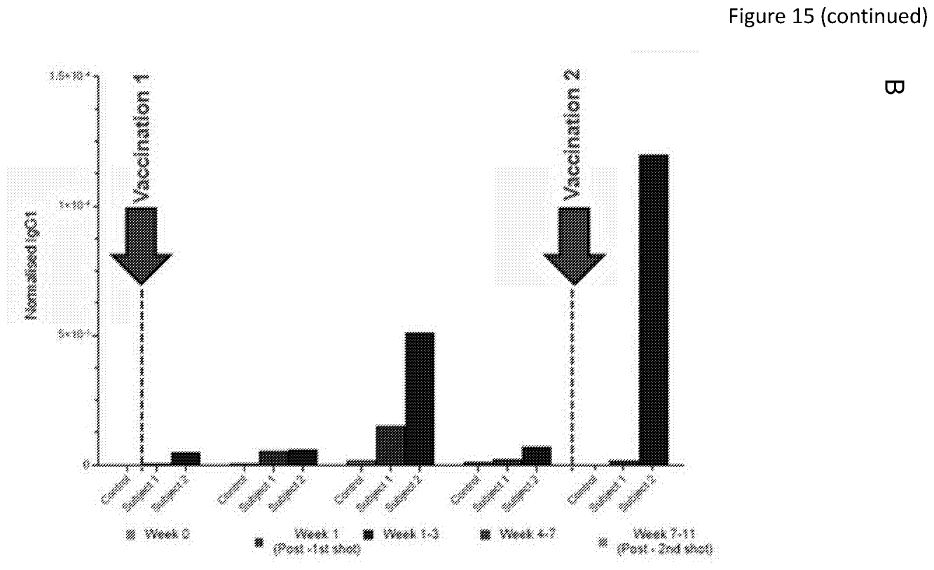

As proof of principle, we also demonstrated that this methodology can also be applied to the much less invasive measurement of antibodies and immune response proteins (than collecting blood serum) using dried blood spots, saliva or saliva adsorbed onto Guthrie card blood collection strips or “lollipops” (supplementary FIG. 15).

Comparison with the Gold Standard S1 RBD Serology Assay and Authentic Live Virus Neutralisation

We compared our assay with the gold standard serology and authentic live virus neutralisation assays at three timepoints, sampling responses 8 weeks after natural infection with Wuhan Hu-1 during the first UK wave and three weeks after the first and second dose vaccination. This enabled us to evaluate and compare our assay after one to three antigen exposures using the same existing peer reviewed, published underpinning datasets analysed in these cohorts (Manisty et al., 2021a; Reynolds et al., 2021b; Reynolds et al., 2020; Reynolds et al, 2021a). We compared results from our assay to those obtained by second generation serology, which measures a total response to spike bait and not a specific antibody (anti-SARS-CoV-2 spike ECLIA assay (Elecsys, Roche Diagnostics), performed by UKHSA, Porton Down).

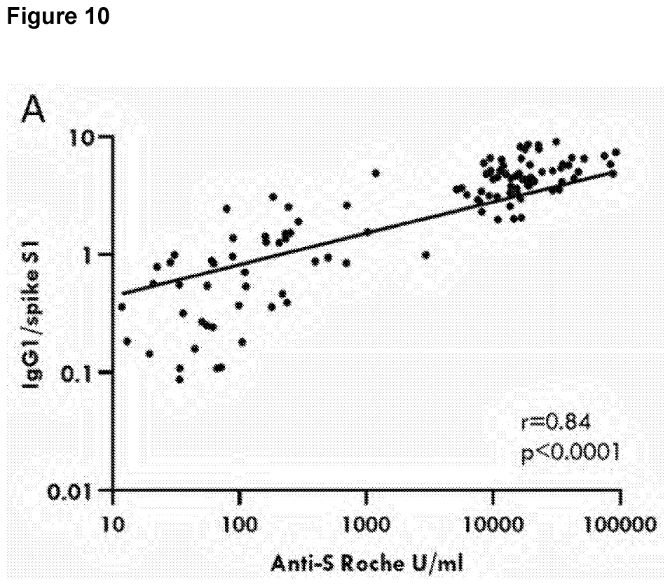

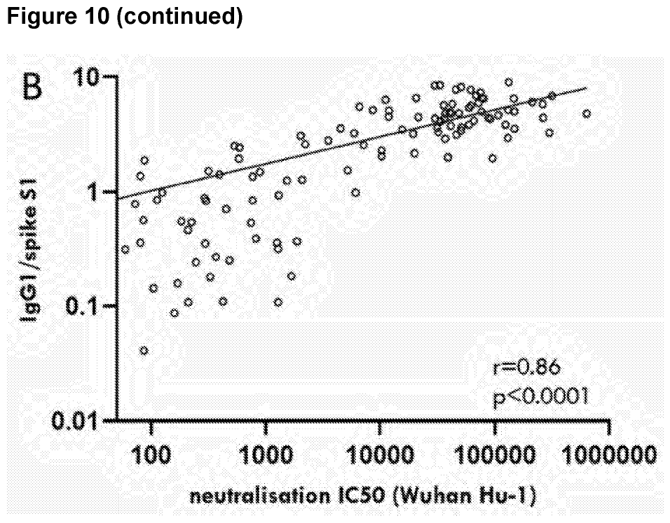

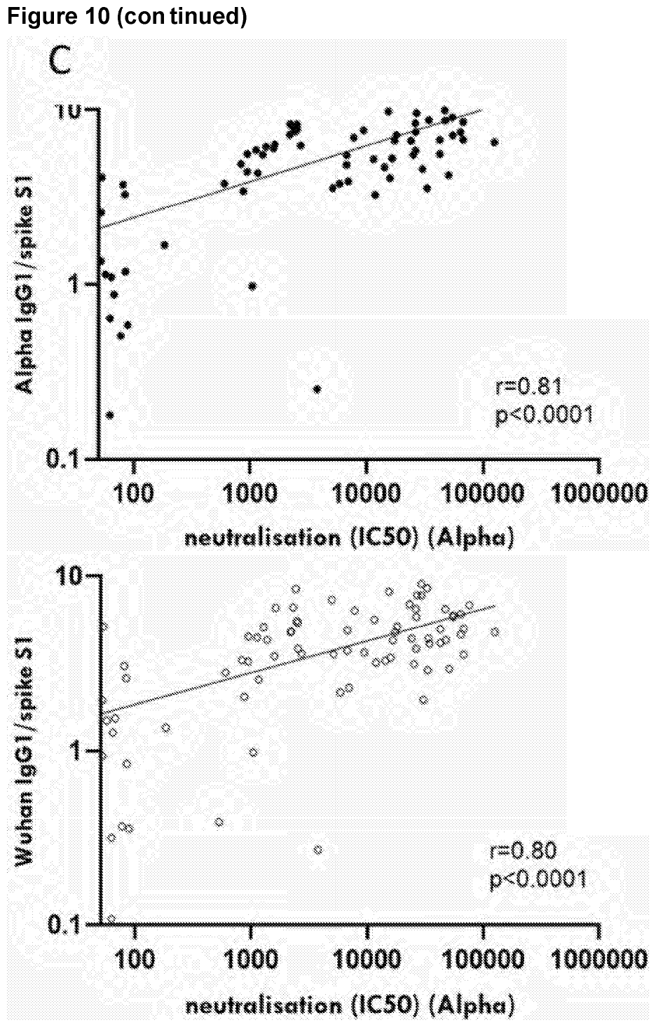

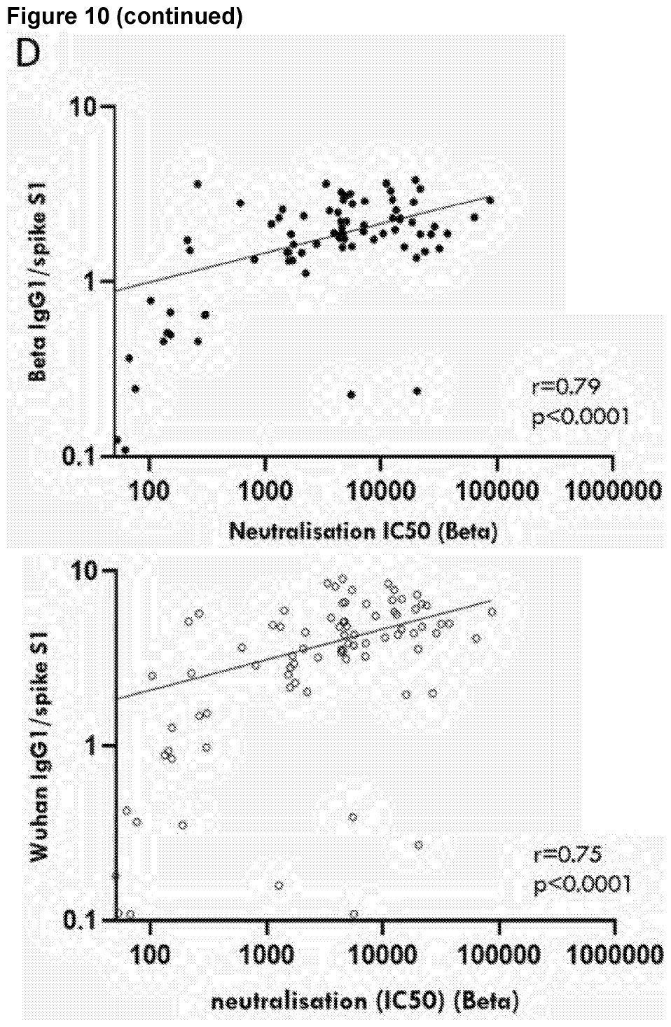

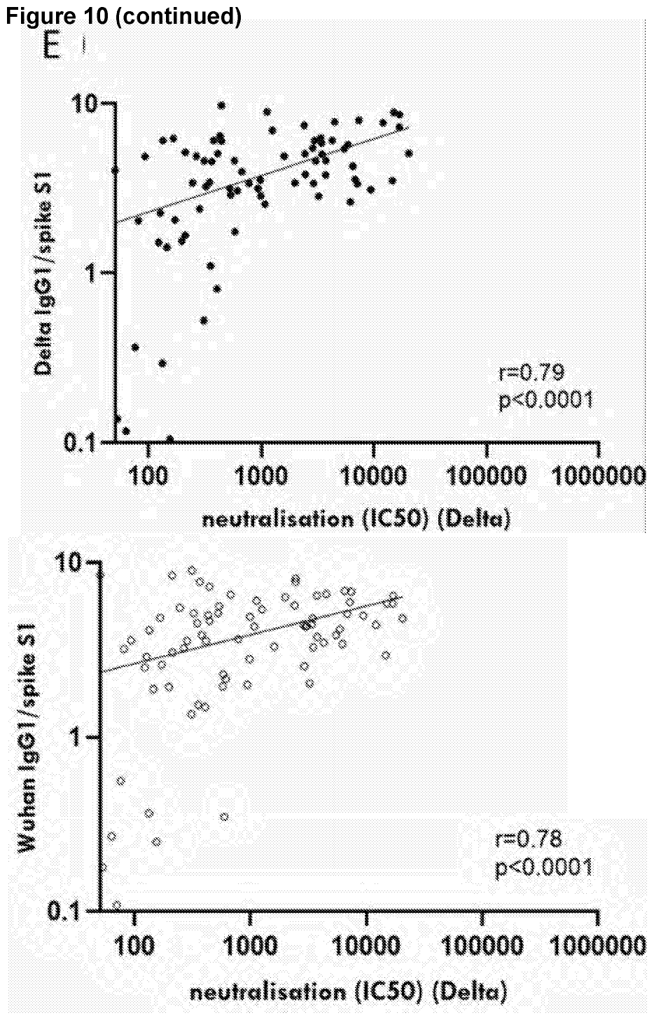

In agreement with other studies (Dogan et al., 2021; Patil et al., 2021), our test also confirmed in most individuals that IgG1 is the main responsive immunoglobulin. FIG. 10A shows that IgG1 correlates well (r=0.84) across a broad range (10 to 100,000 U/ml) with the Elecsys Anti-S assay and requires no dilution and repeat analyses for high titre individuals.

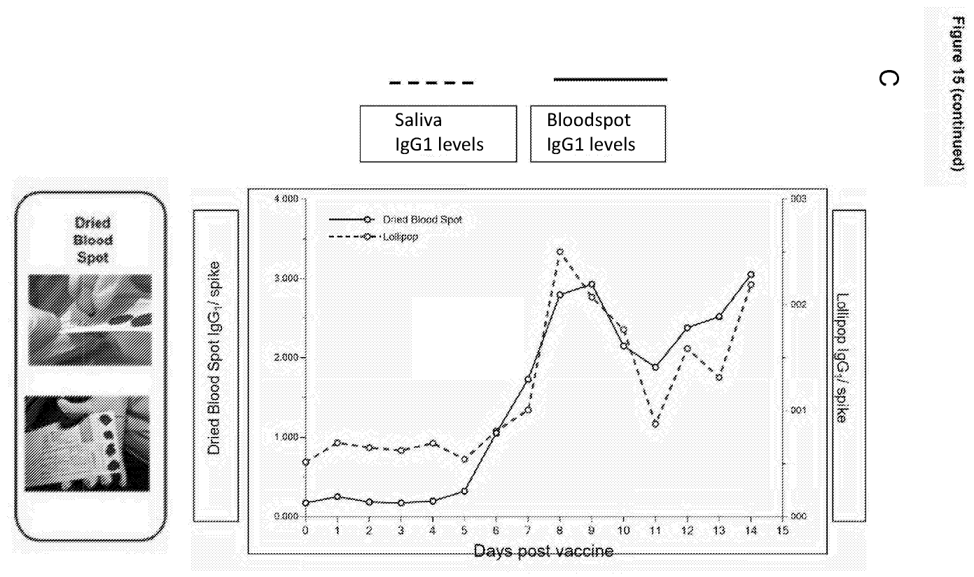

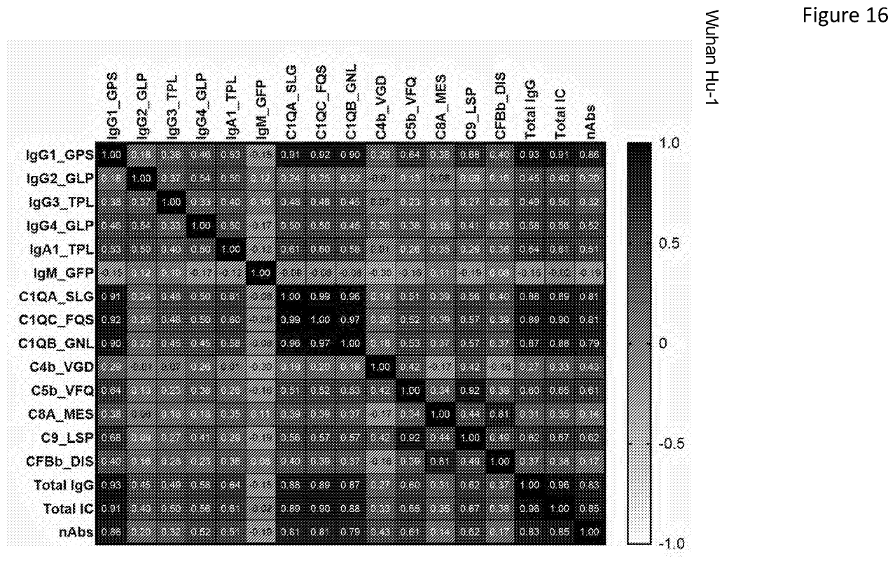

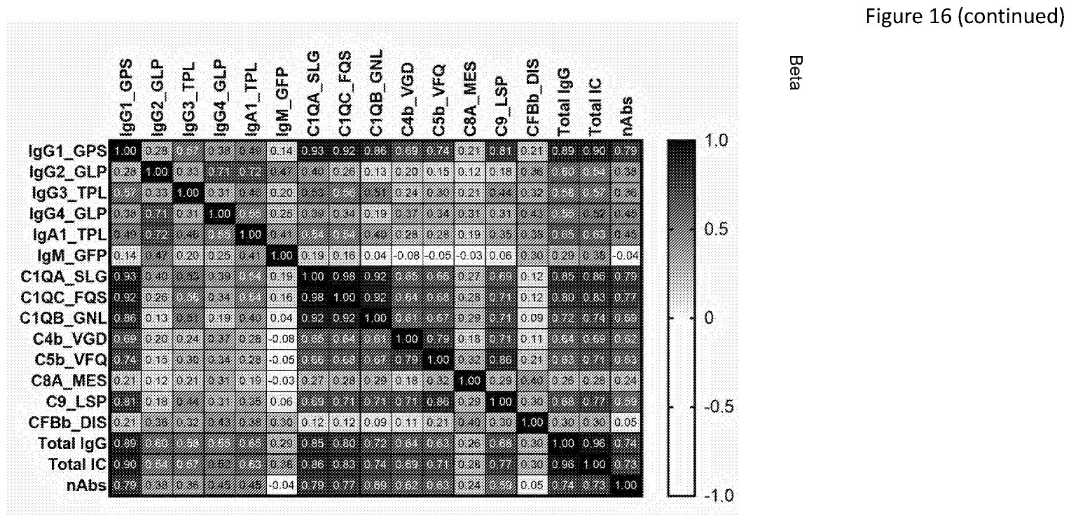

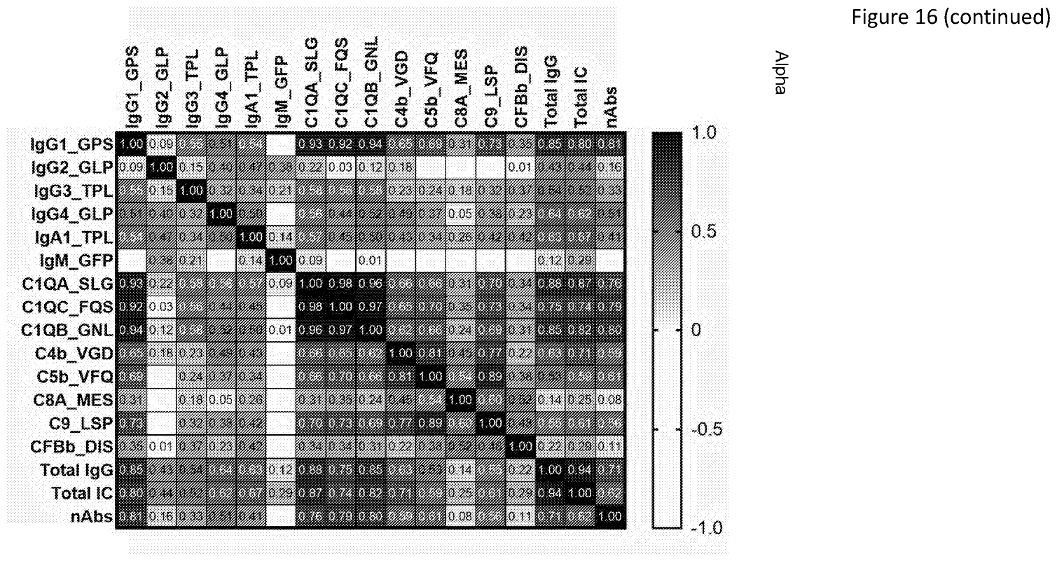

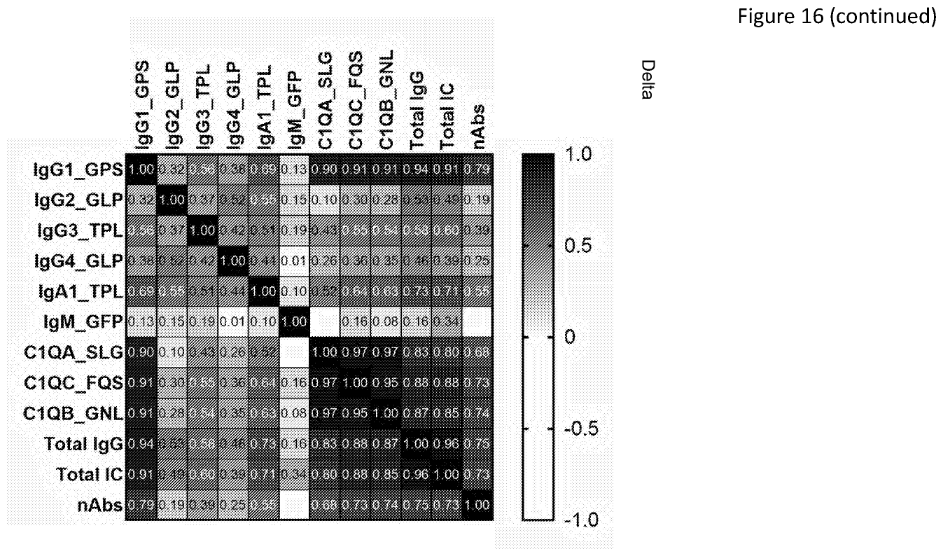

The ability to measure all the components of the immunocomplex, demonstrated that IgG1 showed the strongest correlation with live virus neutralisation data (nAbs) (supplementary FIG. 16) followed by C1q. FIG. 10B-E shows comparison of IgG1 against each S1 variant bait correlates well with corresponding nAbs (r=0.79-0.86). Comparison using only IgG1 Wuhan hu-1 S1 against neutralisation data for each variant shows only slightly less correlation against Alpha, Beta and Delta VoC (r=0.75-0.80). This confirms our high throughput assay demonstrated excellent correlation with conventional neutralising cell-based assays as well and on par with the commercial Roche Anti-S assay.

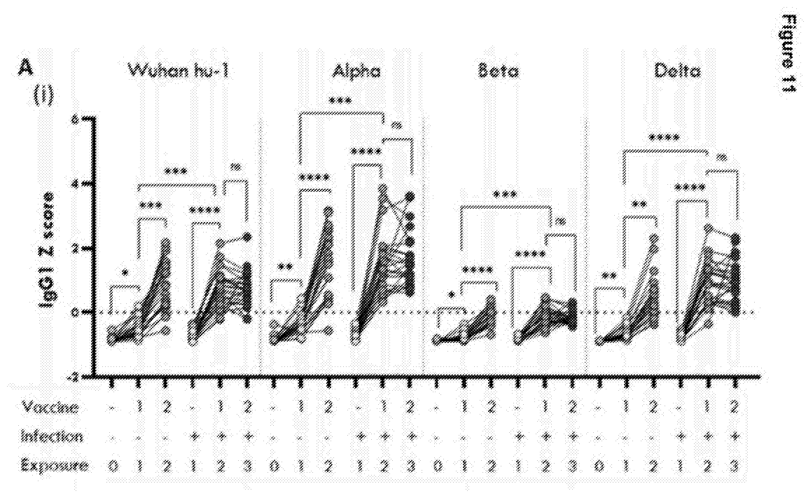

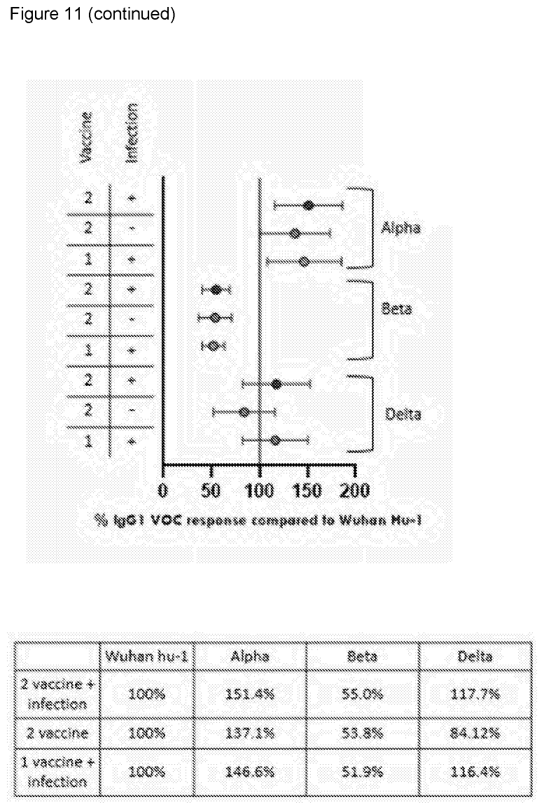

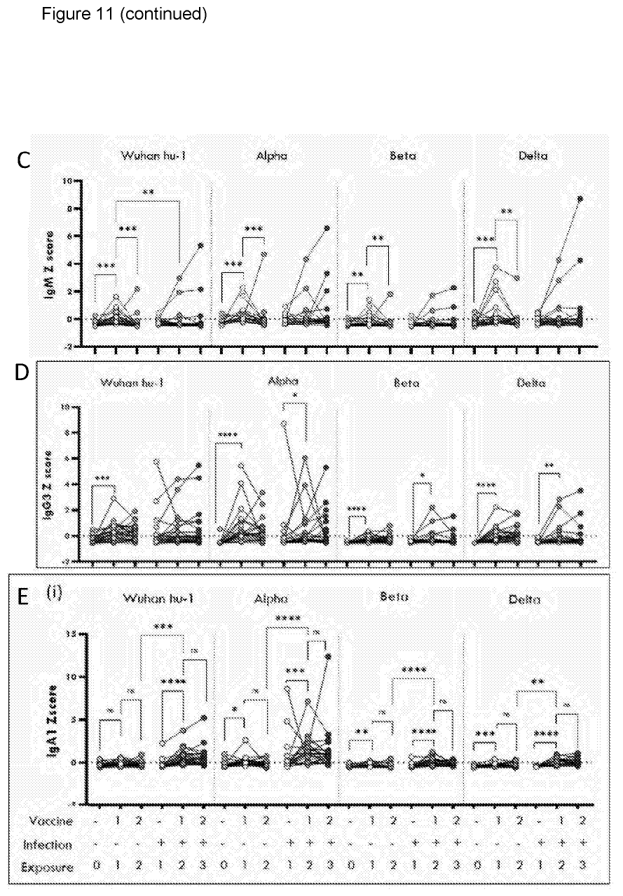

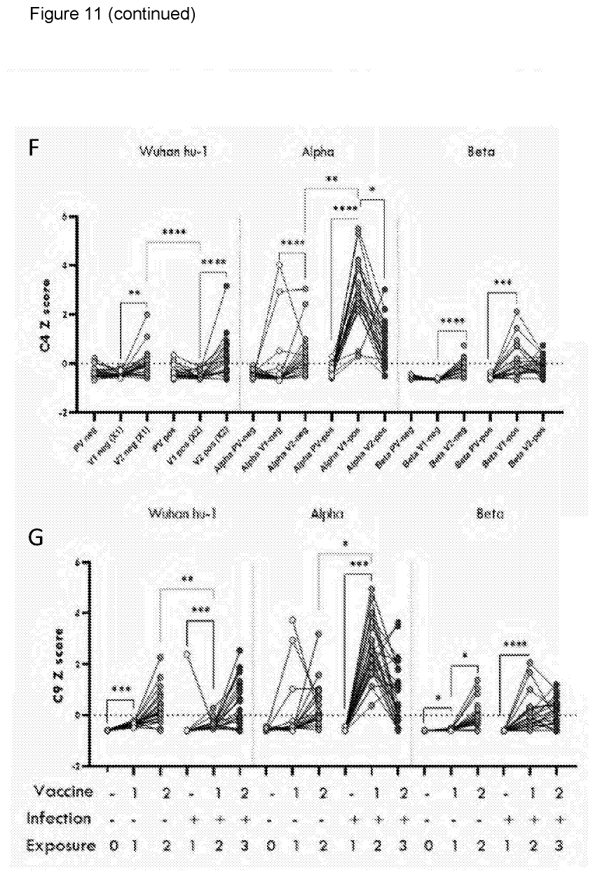

Our results for IgG1 (FIG. 11A) using the LC-MS/MS assay are in line with our published findings that 2 exposures, either via a two dose vaccination protocol, or natural infection and one dose vaccination, have the same effect whereby the second antigen exposure increases anti-Wuhan spike antibody levels on average 3-10 fold (Manisty et al., 2021b; Reynolds et al., 2021b; Reynolds, 2021). In concordance with the gold standard S1-RBD binding and authentic live virus neutralisation assays (Reynolds et al., 2021b; Reynolds, 2021), we found no increase in anti-Wuhan Hu-1 S1 IgG1 responses from the second vaccine dose in previously infected HCW. This suggests an antibody ceiling is achieved at third antigen exposure.

To determine an individual's antibody reactivity against existing and new VoC, the S1 protein from each VoC (Alpha, Beta, Delta, Omicron) were bound in three separate wells to compare a patient's serum antibody binding capability and immune response. IgG1 values were then presented as a percentage of binding capability to the original Wuhan Hu-1 strain and vaccine target, to give a patient a measure of IgG1 reactivity against each VoC (FIG. 11B). We observe more than a 37-51% greater response against the Alpha S1 compared to the Wuhan Hu-1 as described previously and which is slightly enhanced by previous natural infection (Reynolds et al., 2021a). IgG1 response to Delta S1 is similar to Wuhan-Hu-1 (−/+17%) and is also enhanced by previous infection (FIG. 11B). IgG1 response to Beta S1 only elicits a 52-55% response relative to the protection against the Wuhan Hu-1 VoC which is likely due to combined effects of the K417N and N501Y mutations in the Beta spike (Zhou et al., 2021).

Changes in IgM (FIG. 11C) are only observed in the infection naïve group for all variants where the levels increase at first vaccination, but levels are lower at second vaccination confirming IgM only changes in response to first exposure. No significant group changes related to exposure or infection were observed for IgG2 or IgG4 in most individuals. However, some individuals had notable and significantly higher levels of IgG2 and IgG4 (supplementary FIG. 14) and indicate a more personalised medicine approach may be applicable for some individuals. IgG3 is always observed albeit at lower levels and as would be expected (Ferrante et al., 1990) it appears to increase at first exposure in the vaccine naïve group and unlike IgM the levels appear to stay elevated with further exposure. The pre-infection group show a small significant increase at first vaccination only for the VoC (FIG. 11D). Of note are differences in the levels of IgA1 (FIG. 11E). No change is seen in the infection naïve group against the Wuhan Hu-1 bait but an increase is observed for the pre-infection group at first vaccination. However, for the VoC baits after first vaccination we observed a small but significant increase of IgA1 in the infection naive group though this response in the group who had a previous infection was more marked. When comparing to double exposure groups (2 dose vaccination infection naïve vs 1 dose vaccination with prior infection), we see clearly see a greater IgA1 response (p<0.01) in those individuals who had a natural infection (FIG. 11E).

### Understanding Antibody Protection Beyond Immunoglobulins: Determining and Quantitating the Complement Response Against Wuhan Hu-1 and Variants of Concern

Current tests only determine IgG or total response. Using our assay we are able to also determine the significant and important contribution from the complement system. Complement C4 levels demonstrated an average 2-6 fold significant increase against the native Wuhan hu-1. This was observed only at the second vaccination stage in both groups indicating a response to vaccine but interestingly not from exposure to SARS-CoV-2 in natural infection (FIG. 11F). Complement C9 also appears to significantly increase with vaccination first dose against Wuhan-Hu1 by an average 3.8-5.2 fold (FIG. 11G). However, the C4 and C9 response to the Alpha and Beta VoC, respectively, were observed to be markedly different than that to the native Wuhan-Hu1. The response to the Alpha shows that both complement C4 and C9 increase greatly in the pre-infection group after first vaccination (9.6-fold and 93-fold, respectively). However, this response is not as great after a second vaccination with a lower 3.8-fold and 46-fold increase, for C4 and C9 respectively compared to the pre-vaccine group. A similar but lower level response pattern is observed for Beta. This indicates pre-infection elicits greater C4 and C9 against VoC binding after a second antigen exposure.

Complement C1q binding to all variants increases after exposure similar to that of IgG1 (FIG. 12A) but the levels observed are lower against the Beta and Delta. Whilst it is not significant, we did observe less C1q binding at 3^{rd} exposure where binding increased 42-fold after first vaccination but only a 31-fold increase was observed at second vaccination in the pre-infected group. The similar pattern of C1q binding to IgG1 is likely due to the direct interaction of C1q with the fc region of IgG, when compared we observe a significant correlation (r>0.8) between IgG1 and C1q for all variants. However, when comparing correlation between variants, a reduction in the ratio of IgG1 to C1q against the Delta VoC and to a lesser extent against the Beta VoC were observed (FIG. 12B). This suggests potentially a reduced interaction of C1q with IgG1 against Beta and Delta VoC.

Considering the relationship of IgG1 to nAbs (r>0.79 FIG. 10) we also looked at the C1q relationship to NAb levels for each variant (FIG. 12C). Unlike with the IgG1 levels, we did not observe any significant differences in the gradient/ratio of C1q to nAbs when compared to the other variants. This indicates a better association of C1q to nAbs than IgG1 for the Delta VoC.

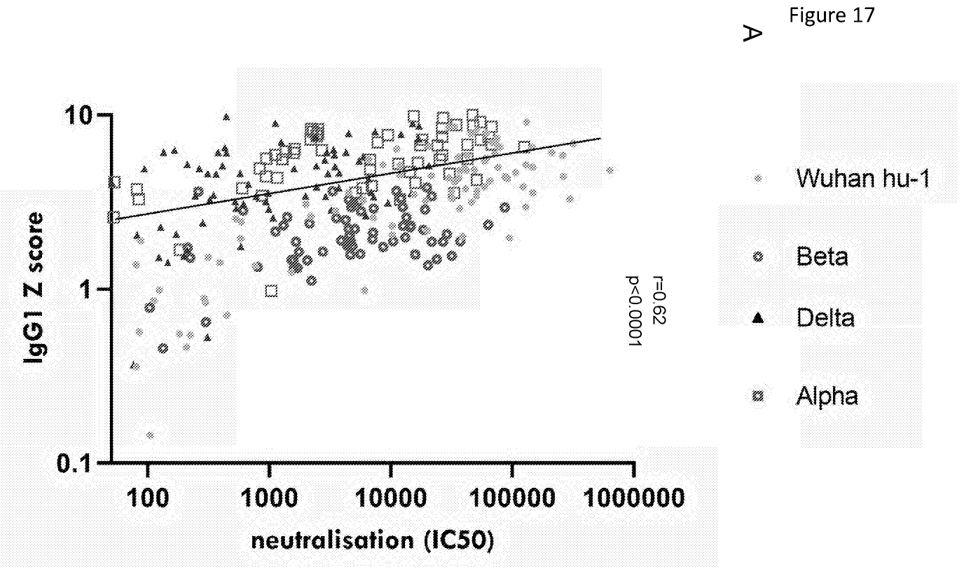

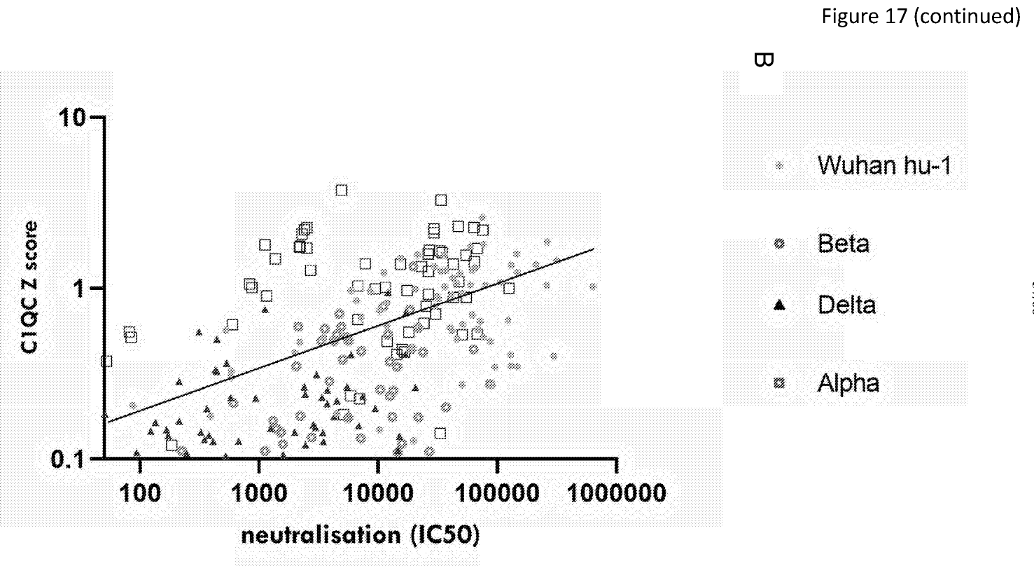

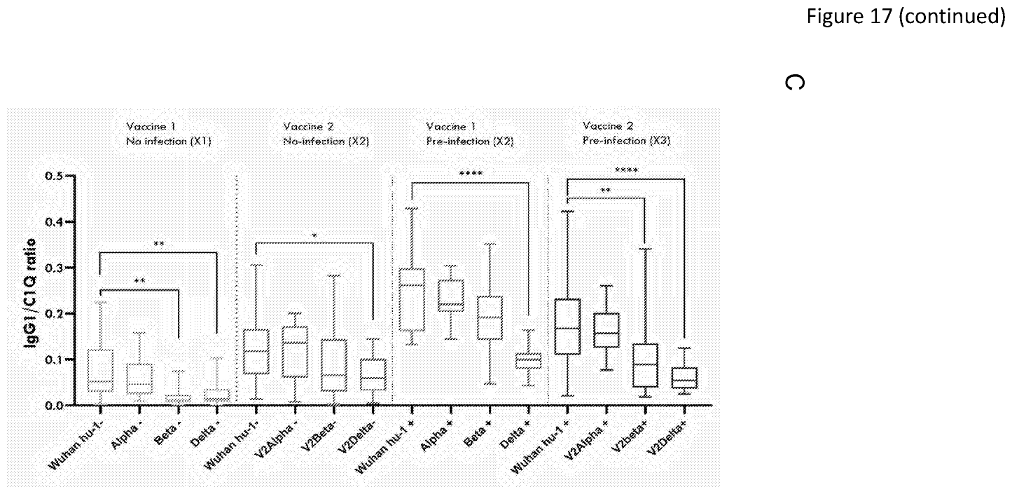

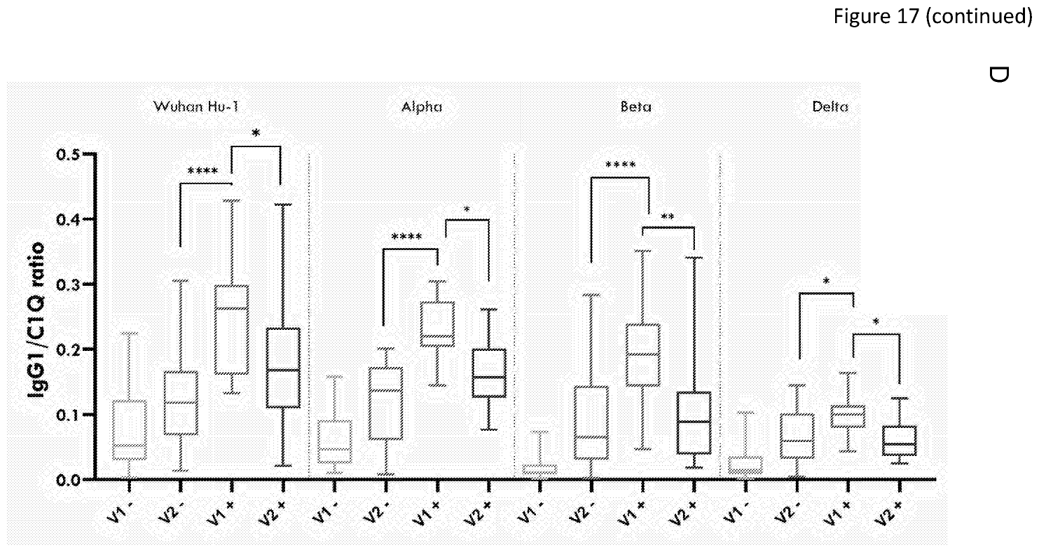

To demonstrate this further, when we combine the nAb data for all variants and then compare with IgG1, and also C1q, we can observe that C1q has an overall stronger correlation (r=0.62 vs 0.71) with nAb than IgG1 (supplementary FIG. 17A-B). If we look at the data as a ratio of C1q to IgG1 across the vaccine groups, we see this ratio is increased with previous exposure to infection (p<0.01 all variants) but this significantly declines at third exposure (p<0.001 all variants) (supplementary FIG. 17C). When comparing across the variants, we see that the Beta and Delta groups show a significant reduced ratio compared to Wuhan Hu-1, with the exception of the second exposure groups where the ratios are normal against the Beta VoC (supplementary FIG. 17C). The significance of this observation is unknown and requires further investigation but could relate to a change in the antibody function or maturation that's affected by prior infection and variant type.

The VoC response compared to Wuhan-hu1 is summarised in FIG. 11H showing an overall reduced response to the Beta VoC although with a greater C9 response at first vaccination. A greater IgM response against Alpha and Delta is detected in the pre-infected groups and is likely due to a first exposure to a variant. These changes likely relate to contribution of non-neutralising antibodies that use complement mediated lysis which can be driven by IgM (17).

Application of the Multiplex Assay to the Omicron Variant with Triple Vaccinated Samples

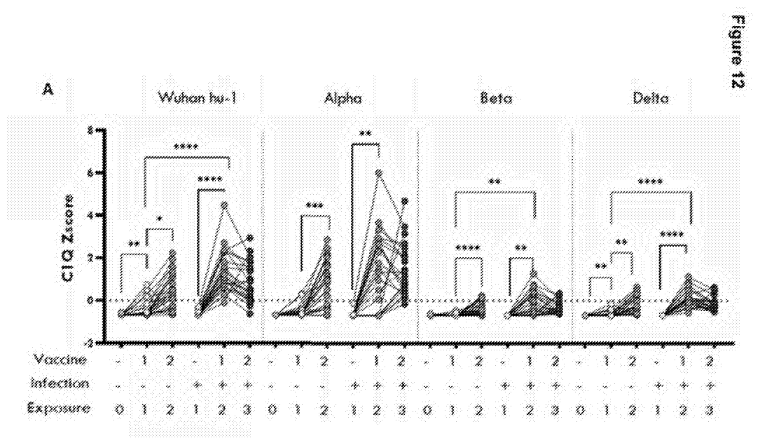

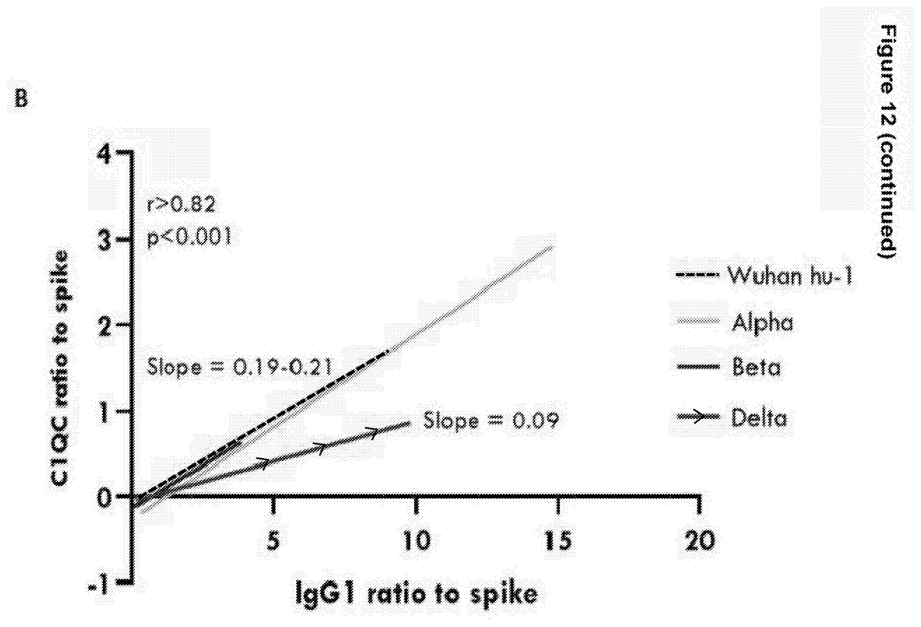

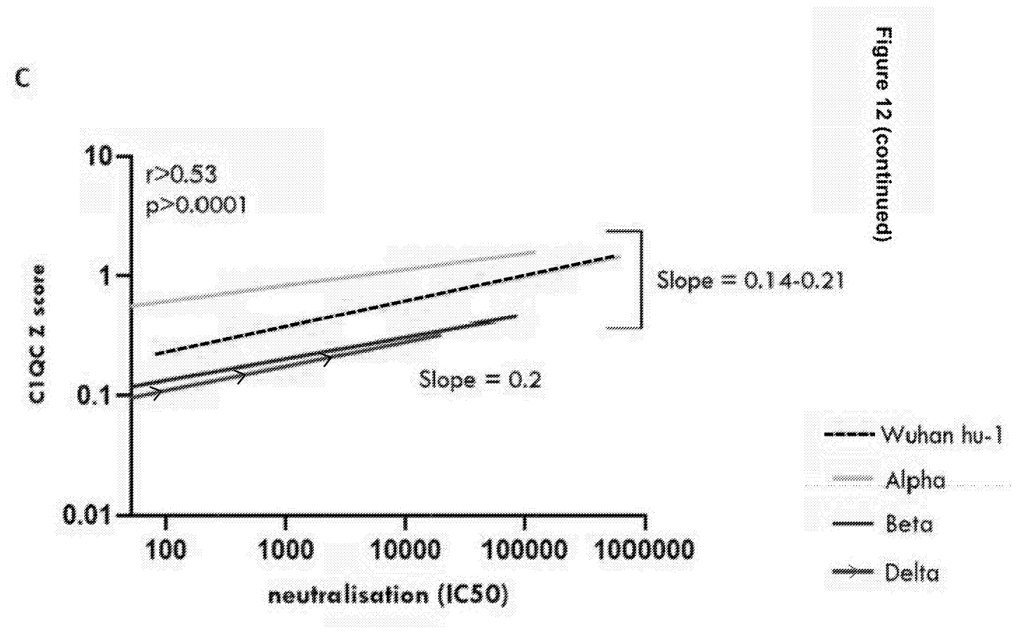

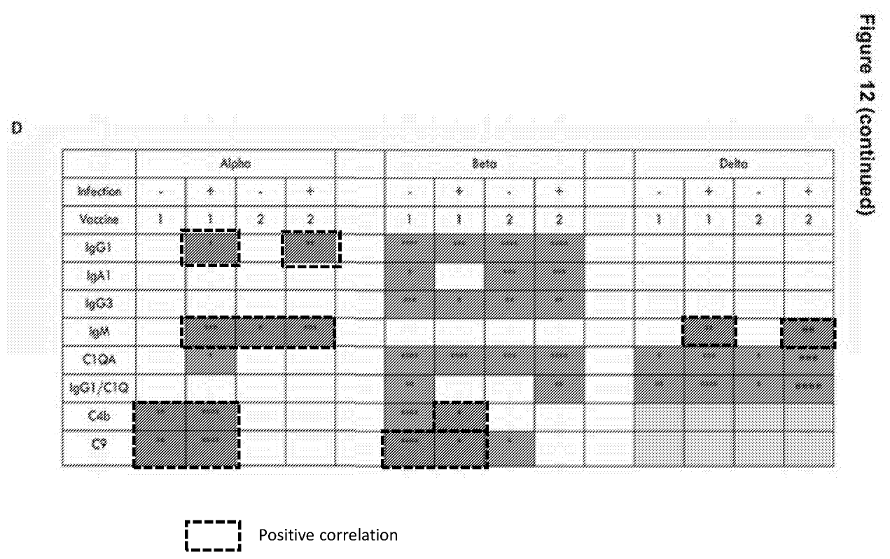

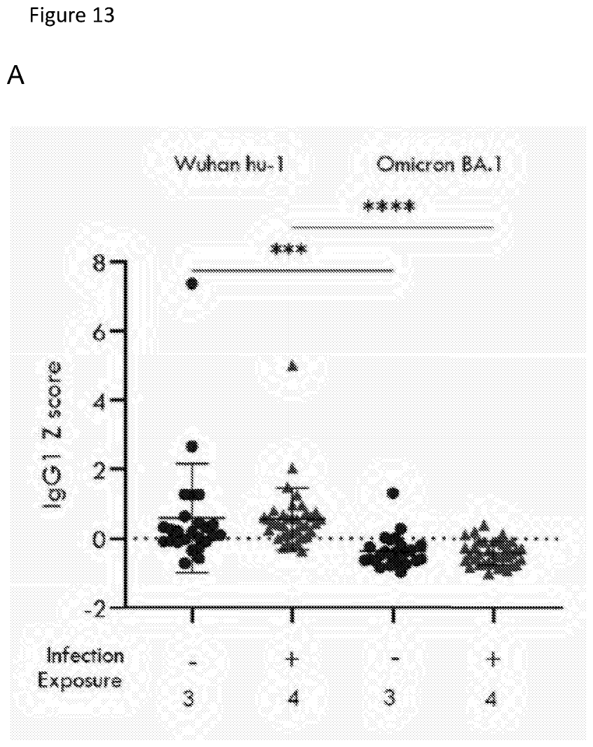

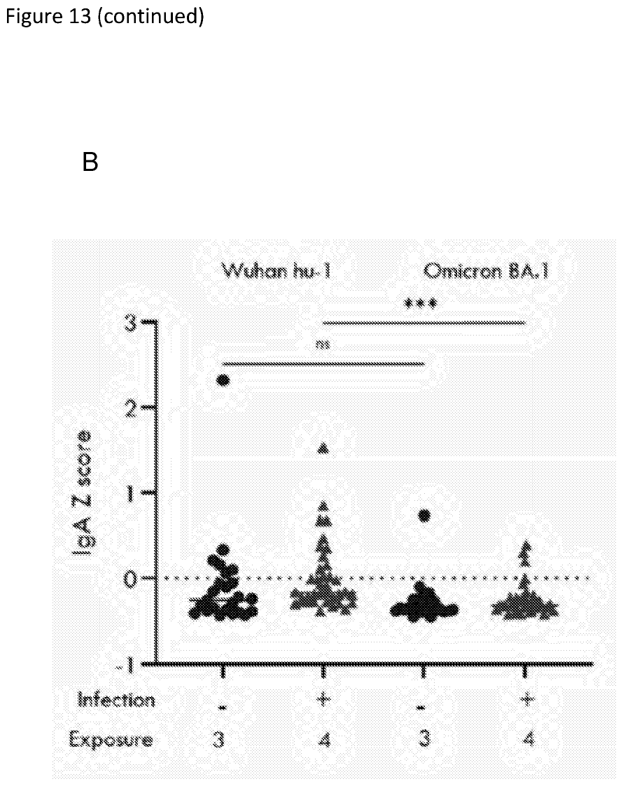

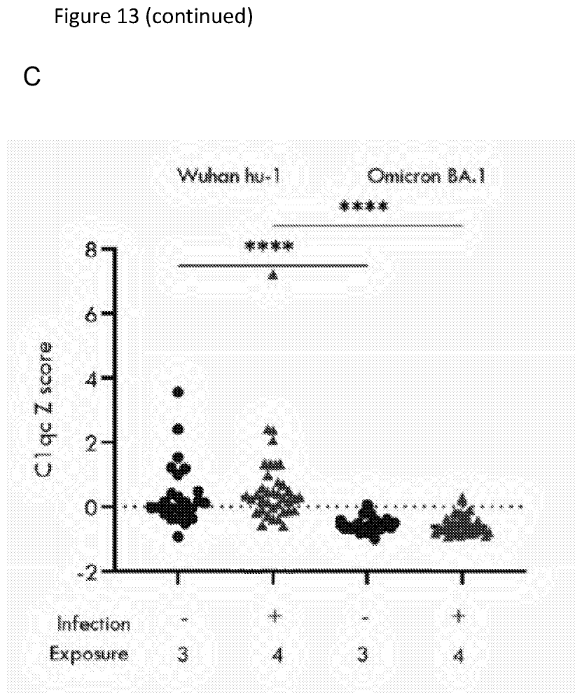

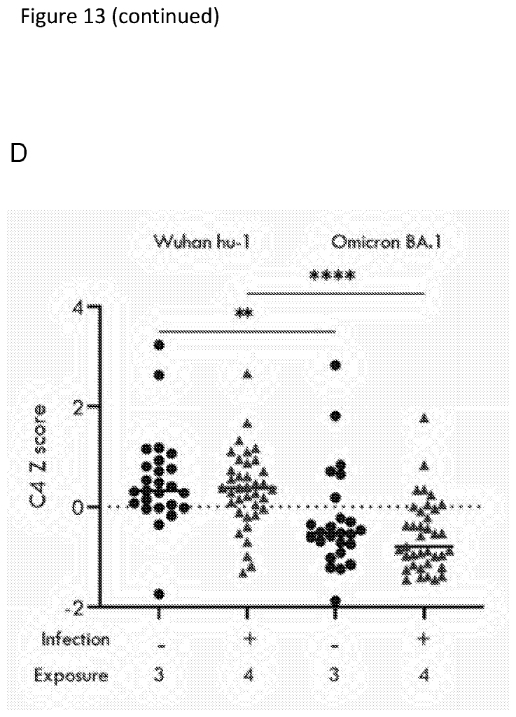

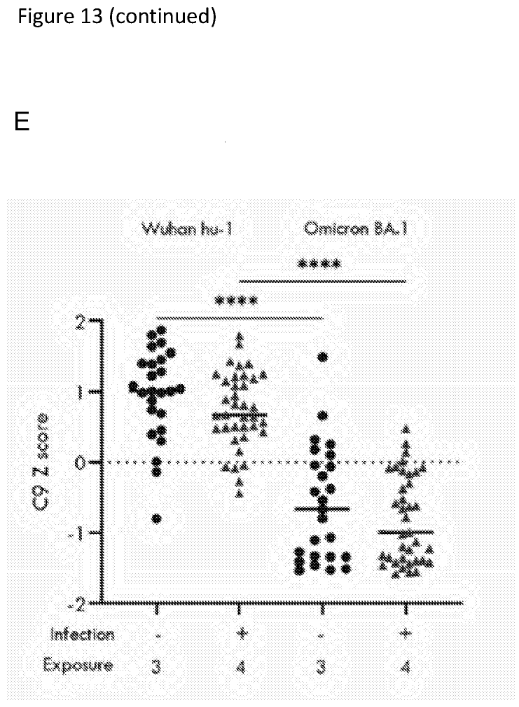

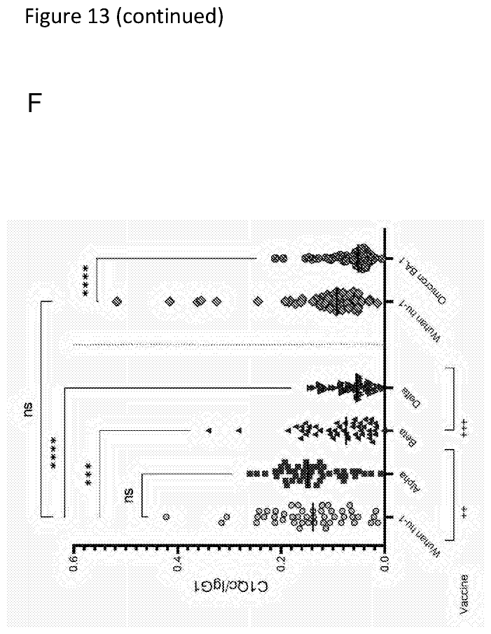

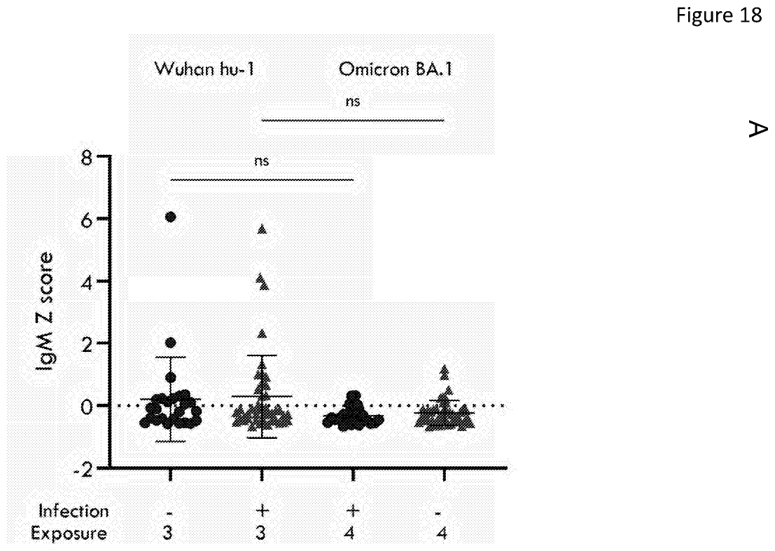

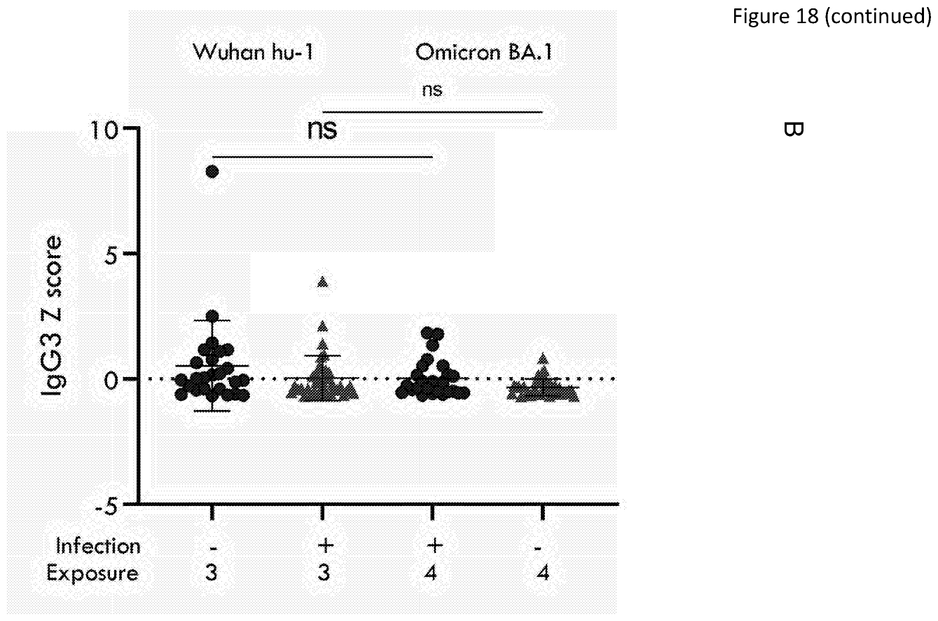

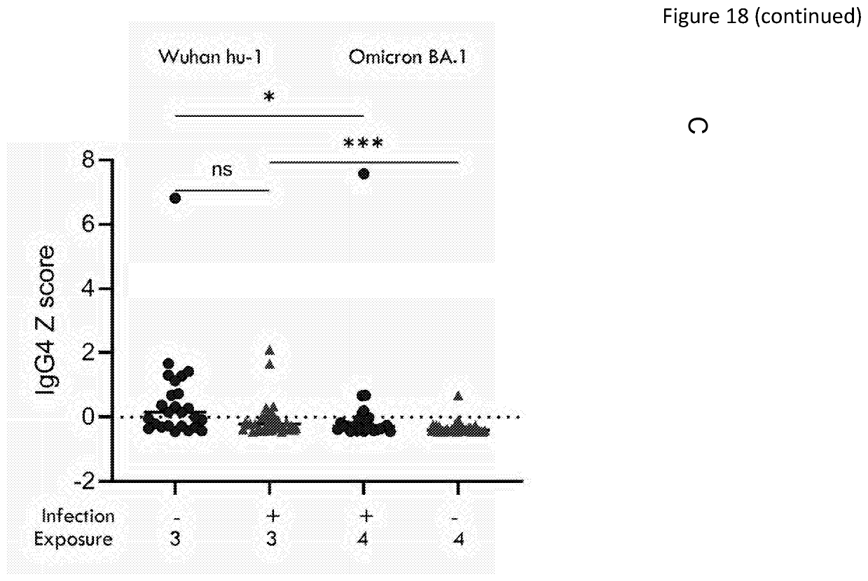

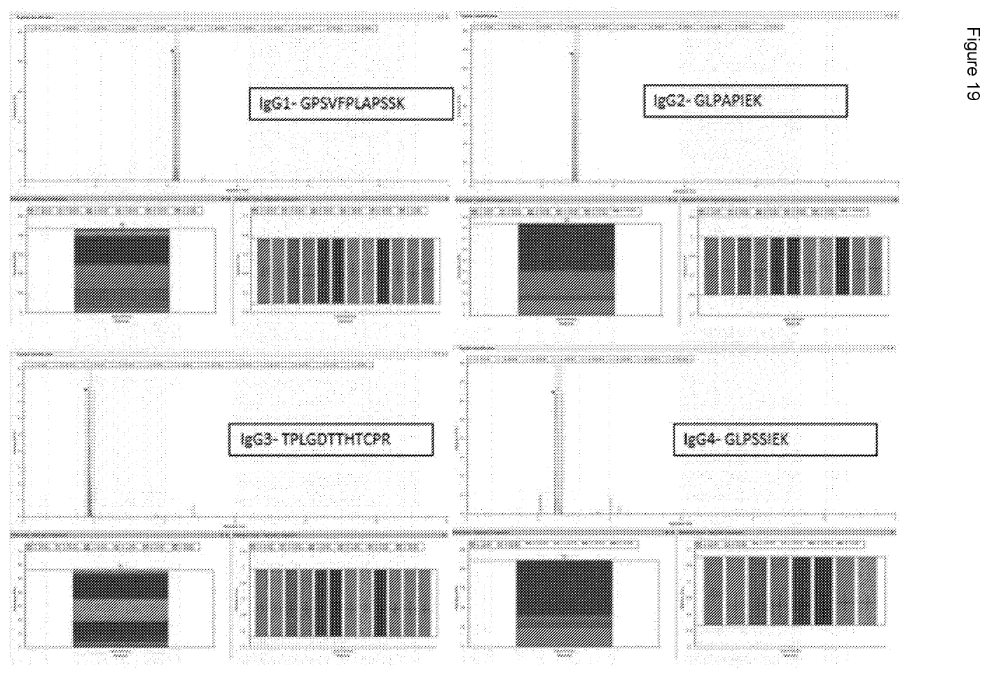

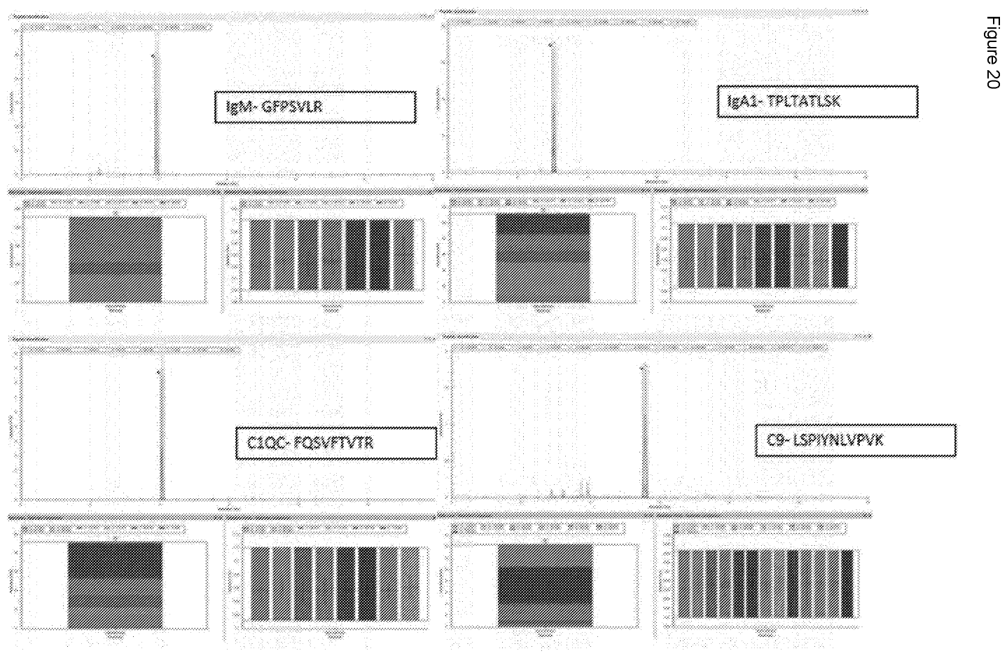

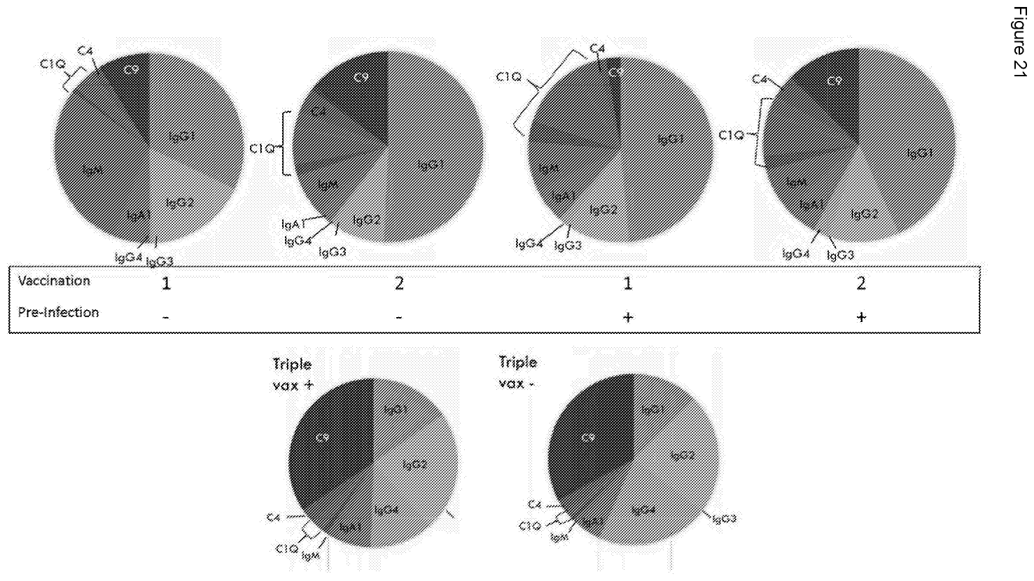

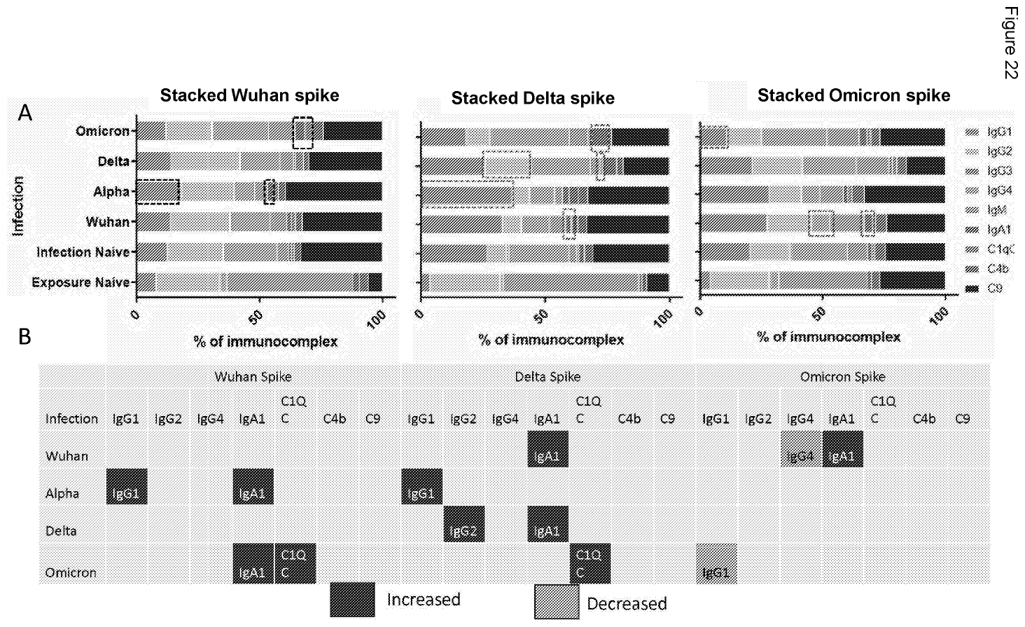

The Omicron variant B.1.1529 emerged in late November 2021, is the most genetically divergent variant to occur (Saxena et al., 2022) and at the time of writing BA.2, is rapidly becoming the main SARS-CoV-2 variant infecting individuals worldwide. Using recombinant spike S1 protein we modified the assay and applied the multiplex assay to a separate cohort of HCW serum samples who had all received a third Pfizer/BioNTech BNT162b2 dose. This cohort of samples were from HCW that and were either infection-naïve or had been previously infected by ancestral Wuhan hu-1, Alpha, or Delta variants. FIG. 13A shows a reduction of IgG1 binding to omicron S1 compared to the Wuhan hu-1 (p<0.001). IgA1 which was typically higher in pre-infected samples also demonstrated a reduced binding to Omicron in the prior infected group (FIG. 13B). No changes were observed for IgG2, IgG3 and IgM though a small degree of reduced binding was observed for IgG4 for both groups (supplementary FIG. 18). The complement proteins C1q, C4 and C9 were all shown to bind with a greatly reduced capacity against omicron S1 protein (FIG. 13C-E). When comparing the ratio of C1q to IgG1 with the other variants we see a lower but non-significant trend with the Wuhan hu-1 after third dose with a significantly reduced ratio for omicron similar to that of the delta VoC (FIG. 13F).

### Example 15—Discussion

Standard SARS-CoV-2 serological assays are becoming less effective as a representation of a ‘correlate of protection’ against variants (Reynolds et al., 2021a) and there is a need for a more informative test to determining this information. This is due to the lack of specificity of current tests which may either measure a total response to bait antigen such as the Roche Elecsys assay or total IgG. A measure of total response of all proteins with affinity for the spike protein will include all other components of the immunocomplex, such as other class immunoglobulins and complement. However, these conventional assays do not tell us how much these immune proteins contribute individually and are extremely important if we are to understand more fully the mechanisms of protection against SARS-CoV-2 variants. Whilst the total response has been adequate for monitoring response to the original Wuhan hu-1, it is no longer adequate against VoC and in populations with an evolving immune response to multiple exposures (Reynolds et al., 2021a).

Our multiplexed approach is capable of looking at all immune reactive proteins individually and demonstrates that other classes of immune proteins, such as IgA and the complement system, also significantly contribute to an individual's overall immune reactivity against VoC, all of which would be missed by conventional testing.

Just looking specifically at IgG1 if we use the Wuhan hu-1 response as a baseline we see a slight significant increase of IgG1 against the, Alpha VoC (FIGS. 10B and 4D) which could be due to a more open conformation of the RBD domain in the Alpha spike (Yang et al., 2021). However, we observe no change for the Delta VoC. In addition, and confirming previous findings, pre-infection has been shown to significantly boost antibody response of the first dose of the vaccination (Anichini et al., 2021; Ebinger et al., 2021; Reynolds et al., 2021b). However, our data indicates that a double vaccine dose in the infection naïve, still does not achieve mean values of those who had pre-infection. Thus the combination of infection plus vaccination may provide a potentially higher immunity versus that of a conventional 2 dose vaccination protocol. This effect is more noticeable for Delta exposure (FIG. 10B) and previous reports have shown that prior infection does reduce the rate of Delta breakthrough infections (Kim et al., 2021).

However contrary to our observation of greater or unaltered IgG1 it has been shown that nAbs are lower against VoC (Reynolds et al., 2021a). This indicates IgG1 is less of a reflection of NAb ability against the VoC. This is likely due to a greater contribution of non-neutralising antibodies against the VoC that can't be differentiated by IgG1 measurement.

The evaluation of bound antibodies and complement fixation beyond just IgG1 in our assay, revealed a surprisingly large degree of heterogeneity in response to exposure with some individuals having completely altered immunoglobulin profiles (supplementary FIG. 14). The relevance of some patients having a lower IgG1, but higher IgG2 and IgG4 response is unknown, but this observation would be overlooked using conventional testing. This could also have relevance in future studies of inter-individual and longitudinal responses to continued exposure of SARS-CoV-2 and vaccines. This ability to define an individual's immunoglobulin profile and determine the variability of an individual's immunological make up, is both intriguing and potentially very important, particularly in different clinical scenarios such as disease severity and mortality (Della-Torre et al., 2021; Patil et al., 2021; Perez-Toledo et al., 2021).

Unlike other assays available, our platform also measures the immunocomplex binding from the lesser well understood but very important complement system. Complement activation can contribute to anti-viral defence by the classical or lectin pathway leading to neutralisation by viral opsonisation and lysis (Jayasekera et al., 2007; Kunnakkadan et al., 2019; Schiela et al., 2018; Vasantha et al., 1988). Previous observations from another study which also looked at other components of the immunocomplex against SARS-CoV-2 RBD domain also highlighted the relevance of C4, C3 and terminal complement complex TCC (or membrane attack complex) deposition in relation to disease severity (Jarlhelt et al., 2021). They confirmed that deposition of C4, C3 and TCC is mediated by antibodies binding to epitopes on RBD, as complement deposition was almost completely absent post depletion of RBD antibodies in convalescent plasma samples. This was thought to be driven by IgG as complement increased with IgG in response to disease severity. However, against the variants our findings using S1 spike protein show complement C4 and C9 is greater against variants at initial exposure. This could be due to contribution of non-neutralising antibodies as studies on parainfluenza virus show that complement mediated neutralisation is likely to be a mechanism of non-neutralising antibody action driven by IgM (Vasantha et al., 1988). Our findings corroborate with this as we observe a greater IgM response against Alpha and Delta, which is also accompanied by greater C4 and C9 binding (FIG. 12D). However, there is likely the presence of non-neutralising IgG1 as well due to the fact that IgG1 levels are unchanged against the Alpha and Delta variants (FIG. 10) but there are reduced neutralising antibodies (Reynolds et al., 2021a).

One of the more interesting findings in the analyses of the immunocomplex was that of the role of C1q. C1q directly interacts with the fc portion of immunoglobulins and is required for initiation of the complement cascade. In our analysis C1q binding appears to behave independently from the other complement components (FIG. 12) and showing a reduction in binding against the Beta and Delta VoC (FIG. 12A) as opposed to the increase we observe for C4 and C9 binding. Interestingly, for the Delta VoC, C1q was reduced relative to IgG1 but not relative to nAbs. This indicates that C1q could be a better surrogate indicator of NAb protection against SARS-CoV2 variants than IgG1. Previous work has shown that heat-inactivation, which would inactivate complement appears not to affect IgG reactivity to the RBD domain (Amanat et al., 2020), but may affect its neutralisation capability (Pastorino et al., 2020). This confirms that complement whilst is not essential may have a role in neutralisation. Further weight to this observation comes from Melhop et al, who showed that C1q increases the potency of antibodies against West Nile virus by modulating the stoichiometric requirements for neutralisation (Mehlhop et al., 2009). Therefore, it is possible that complement could contribute potentially to protection against VoC by C1q augmenting neutralising antibodies and non-neutralising antibodies using the classical complement pathway for viral opsonisation. If indeed C1q binding is more specific to nAbs then the altered ratio between C1q to IgG1 for the Delta VoC, which we observed in this study again could be explained by a greater proportion of non-neutralising IgG1 antibodies that are present. This finding of the C1q association with nAbs merits further investigation to confirm if the IgG1 to C1q relationship could be an indirect way of determining neutralising and non-neutralising antibody responses to VoC.

The most recent B.1.1529 (Omicron) response was also evaluated in a separate triple vaccinated cohort of HCW and compared to the wild type Wuhan hu-1. Unlike with other VoC a significantly reduced response was observed for IgG1, as well as IgA1, IgG4 and complement binding. This is in accordance with previous work that confirmed neutralisation is reduced against the Omicron VoC (Dejnirattisai et al., 2022) even after a booster vaccination (Yu et al., 2022). In addition a lack of increased IgM and C4 and C9 complement response that we see for other variants was not apparent for Omicron. Considering that Omicron is the most genetically distinct VoC with over 30 mutations in its spike protein (Wang and Cheng, 2022) it is likely there is also a lack of non-neutralising antibodies that recognise the spike due to the greater number of mutations. This may explain the reduced C4 and C9 complement which may be associated to non-neutralising antibody mediated viral opsonisation by complement (Vasantha et al., 1988).

In this study, our data shows only a snapshot of the antibody response approximately 3 weeks after vaccination in non-hospitalised healthcare workers. Further studies to characterise the effect over time, infection from different variants and infection severity, and how the ‘immunocomplex signature’ changes with age, would give us a greater understanding of the evolving antibody mediated immune response to SARS-CoV-2. Our findings also uncover an area of antibody mediated immunity that is little understood and complement function is far more complex than what we can relay in our study. One of the limitations with our assay is that we are not able to determine functional complement activation, although precise quantitative detection may be able to funnel further investigation. Our approach using multiplex LC-MS/MS could provide a valuable platform to better enable research in this area. Whilst the LC-MS/MS multiplex assay is a research standard assay, it was designed so it can be easily translated for use in a clinical laboratory setting (Smit et al., 2021). The information obtained will allow us to understand in greater detail an individual's antibody protection or be used in vaccine design. Furthermore this ‘Bait, Capture and Mass Spec’ approach, can also have applications beyond SAR-CoV-2 for other infectious diseases, or even immune response to novel treatments and autoimmunity.

### Example 16—Experimental Model and Subject Design

### Ethics Statement

Human sera were obtained from the COVIDsortium Healthcare Workers bioresource (Manisty et al., 2021a; Reynolds et al., 2021b; Reynolds et al., 2020; Reynolds, 2021; Treibel et al., 2020) which is approved by the ethical committee of UK National Research Ethics Service (20/SC/0149) and registered on ClinicalTrials.gov (NCT04318314). The study conformed to the principles of the Helsinki Declaration, and all subjects gave written informed consent.

COVIDsortium Healthcare Worker Participants SARS-CoV-2 infection (by the Wuhan Hu-1 strain) of study participants was determined by baseline and weekly nasal RNA stabilizing swabs and Roche Cobas® SARS-CoV-2 reverse transcriptase polymerase chain reaction (RT-PCR) test as well as baseline and weekly serology using the EUROIMMUN Anti-SARS-CoV2 enzyme-linked immunosorbent assay (ELISA) and ROCHE Elecsys® Anti-SARS-CoV-2 electrochemiluminescence immunoassay (ECLIA). Antibody ratios >1.1 were considered test positive for the EUROIMMUN SARS-CoV-2 ELISA and >1 was considered test positive for the ROCHE Elecsys anti-SARS-CoV-2 ECLIA following Public Health England evaluation (Manisty et al., 2021a; Manisty et al., 2021b; Reynolds et al., 2021b; Treibel et al., 2020).

The previously reported^{13,14} cross-sectional, case-controlled vaccine sub-study (n=51) collected samples at a mean/median timepoint of 22d and 20d after administration of the first and second dose of the mRNA vaccine, BNT162b2. This vaccine sub-study recruited HCW previously enrolled in the 16-18 week sub-study (Manisty et al., 2021b). This included 25 HCW (mean age 44 yr, 60% male) with previous laboratory defined evidence of WT SARS-CoV-2 infection and twenty-six HCW (mean age 41y, 54% male) with no laboratory evidence of SARS-CoV-2 infection throughout the initial 16-week longitudinal follow up. Neutralising antibody and RBD ELISA data obtained by ROCHE Elecsys anti-SARS-CoV-2 ECLIA following Public Health England (PHE) has been previously published (Manisty et al., 2021a; Manisty et al., 2021b; Reynolds et al., 2021b; Reynolds et al., 2020; Treibel et al., 2020).

### Example 17—Method Details

### SARS2-CoV-2 Immunocomplex Assay—Bait Capture

Ninety six well microtitre plates (Waters Corp) were coated with either Wuhan Hu-1 SARS-CoV-2 spike protein (S1) (Genscript Z03501), Alpha VOC B.1.1.7 SARS-CoV-2 (2019-nCoV) Spike S1 (HV69-70 deletion, N501Y, D614G)-His Recombinant Protein (Sino biological 40591-V08H7), Beta VOC B.1.1351 SARS-CoV-2 Spike protein (S1, E484K, K417N, N501Y, His Tag (Genscript Z03531-1), Delta VOC B.1.617.2 (T19R, G142D, E156G, 157-158 deletion, L452R, T478K, D614G, P681R) Protein (His Tag) (Sino biological 40591-V08H19) or Omicron (B.1.1.529/Omicron) Spike Glycoprotein (S1), Sheep Fc-Tag (HEK293) A67V, H69del, V70del, T95I, G142D, V143del, Y144del, Y145del, N211del, L212I, ins214EPE, G339D, S371L, S373P, S375F, K417N, N440K, G446S, S477N, T478K, E484A, Q493R, G496S, Q498R, N501Y, Y505H, T547K, D614G, H655Y (Native antigen REC32006-100). S1 protein was diluted to 50 μg/ml in PBS and 10 μl added to the bottom of each well. Wells were topped with 140 μl of sodium carbonate/bicarbonate buffer 100 mM at pH 9.6 and then incubated for 12-16 hours at 4° C. All further incubations were performed at room temperature (RT) unless stated otherwise. Supernatant was carefully tipped from the wells. Wells were washed with 200 μl of PBS and then incubated for 1 hr with 200 μl of blocking solution consisting 1 mg/ml of horse myoglobin (Sigma UK) in PBS followed by 3 washes with PBS. Plate wells were stored with PBS and kept at 4° C. until used.

Serum samples were diluted 1:10 in 0.05 mg/ml horse myoglobin (Sigma UK) in PBS and added to S1 protein coated wells for 1 hr at 37° C. Sample was carefully removed and wells were washed once with 200 μl 0.05% Tween 20 in PBS and then 3 times with PBS. For saliva 75 ml of neat saliva was diluted 1:1 in 0.05 mg/ml horse myoglobin in PBS and added to baited wells.

Dried blood spots: 6 mm DBS spots were punched into a 2 ml micro tube and extracted using 175 μl of 0.05 mg/ml Horse myoglobin solution in PBS for 1 hour on the shaker. Samples were centrifuged at max rpm on benchtop centrifuge for 10 min. An aliquot 150 μl per reaction was added to the baited well for 1 hr.

Dried Saliva spot (lollipop)—6 mm saliva spots were punched into a 2 ml micro tube and extracted using 250 μl of 0.05 mg/ml Horse myoglobin solution in PBS for 1 hour on the shaker. Centrifuge at max rpm on benchtop centrifuge for 10 min. An aliquot of 150 μl per reaction was added to the baited well for 1 hr.

### Immunocomplex Protein Digestion

Seventy microliters of 0.5% Sodium deoxycholate in 50 mM Ammonium Bicarbonate buffer was added to each well followed by 3 μl of DTT solution (DL-Dithiothreitol (DTT)—162 mM in 0.5% sodium deoxycholate/50 mM Ammonium bicarbonate buffer). Plates were capped and incubated at 85° C. for 15 min with shaking (750 rpm). The plate was left to cool to room temperature before 6 μl of 162 mM Iodoacetamide (IAA) in 0.5% Sodium deoxycholate/50 mM Ammonium bicarbonate buffer was added. Plates were capped and brief shaken and incubated at room temperature for 30 min. Five microliters of trypsin (Sigma) (1 mg/ml in 50 mM Acetic Acid) was added and incubated at 45° C. for 30 mins. Digestion was halted by addition of 5 μl of 6% TFA and mixed well. Plates were centrifuged for 20 min at 4000 g at 10° C. Fifty microliters are aliquoted into a fresh plate and analysed by LC-MS/MS.

### Targeted LC-MS/MS Analysis

Digested samples were injected onto a Waters 50 mm UPLC Premier® C18 1.7 μm, 2.1×50 mm column operating at 45° C., for chromatographic separation. Mobile phase A consisted of: 0.1% formic acid in water and B: 0.1% formic acid in ACN, pumped at a flow rate of 0.3 mL min-1. The starting conditions of 5% B were kept static for 0.1 minutes, before initialising the linear gradient to elute and separate peptides over 7.7 minutes to 40% B. B was linearly increased to 80% over 0.2 minutes and held for 1 minute to wash the column before returning to the initial conditions followed by equilibration for 1 minute prior to the subsequent injection. The LC system was coupled to a Waters Xevo-TQ-S triple quadrupole mass spectrometer for multiple reaction monitoring (MRM) detection in positive electrospray ionisation mode. The capillary voltage was set to 2.8 kV, the source temperature to 150° C., the desolvation temperature to 600° C., the cone gas and desolvation gas flows to 150 and 800 L hour-1 respectively. The collision gas consisted of nitrogen and was set to 0.15 mL min-1. The nebuliser operated at 7 bar. The cone energy was set to 35 V and the collision energies varied depending on the optimal settings for each peptide. Transition information of each peptide is given in supplementary table 1.

### Authentic Virus Neutralisation Assay

SARS-CoV-2 microneutralisation assays were previously reported (Reynolds et al., 2021a; Reynolds et al., 2021b). VeroE6 cells were seeded in 96-well plates 24 h prior to infection. Duplicate titrations of heat-inactivated participant sera were incubated with 3×10⁴ FFU SARS-CoV-2 virus (TCID100) at 37° C., 1 h. Serum/virus preparations were added to cells and incubated for 72 h. Surviving cells were fixed in formaldehyde and stained with 0.1% (wt/vol) crystal violet solution (crystal violet was resolubilised in 1% (wt/vol) sodium dodecyl sulphate solution). Absorbance readings were taken at 570 nm using a CLARIOStar Plate Reader (BMG Labtech). Negative controls of pooled pre-pandemic sera (collected prior to 2008), and pooled serum from neutralisation positive SARS-CoV-2 convalescent individuals were spaced across the plates. Absorbance for each well was standardised against technical positive (virus control) and negative (cells only) controls on each plate to determine percentage neutralisation values. IC50s were determined from neutralisation curves. All authentic SARS-CoV-2 propagation and microneutralisation assays were performed in a containment level 3 facility.

### Example 18—Quantification and Statistical Analysis

### Data Analysis

Raw LC-MS/MS data was analysed using Skyline open source software (https://skyline.ms/project/home/software/Skyline/begin.view). Peptide identifications were determined from prior analysis of digested serum and immunoglobulin standards (Invitrogen) by a minimum of 6 transitions and matched to in-silico spectral library (Prosit) for additional confirmation. Two optimal transitions were used for final MRM analysis. Peptide abundance data were normalised to S1 peptide FASVYAWNR which was present in all variants. For comparison analysis between variant assays ratio values were normalised by Z-Score. Exported data were analysed using Microsoft Excel and Graphpad Prism v9.

Linearity response of IgG up to 250 mg/ml (r>0.99) was confirmed using a calibration curve using IgG protein standard (Sigma, UK) the maximum observed sample IgG1 value was 157.23 ug/ml well within linear range. LOD and LOQ values (supplementary table 1) were determined by proportion of each IgG subclass of standard. High QC and LQC serum was obtained from vaccinated volunteers.

Immunoglobulin CVs were below 30% for all variants apart from low levels for IgG3 and 4. Complement proteins were only detectable in the HQC and showed freeze thaw instability of C4-C9. Therefore complement data for the later emerging Delta variant is not shown.

### Statistics

For comparison of vaccination groups nonparametric ANOVA (Kruskal Wallis) were used to determine significance. For correlation analyses nonparametric spearman test was used to determine significance and r value.

TABLE 9
List of LC-MS/MS assay peptide sequences and transitions.

| | | precursor. | product. | cone_ | collision | ion_ | Sequence_ |
|---|---|---|---|---|---|---|---|
| Protein | Sequence | mz | m_z | voltage | energy | name | SEQ ID NO |
| C4b | VGDTLNLNLR | 557.81 | 1015.55 | 35 | 20 | y9 | 40 |
| C4b | VGDTLNLNLR | 557.81 | 629.37 | 35 | 20 | y5 | 40 |
| C4BPA | YTCLPGYVR | 564.78 | 864.44 | 35 | 20 | y7 | 62 |
| C4BPA | YTCLPGYVR | 564.78 | 591.32 | 35 | 20 | y5 | 62 |
| C5b | VFQFLEK | 455.76 | 811.43 | 35 | 16 | y6 | 48 |
| C5b | VFQFLEK | 455.76 | 664.37 | 35 | 16 | y5 | 48 |
| C8A | MESLGITSR | 497.26 | 733.42 | 35 | 17 | y7 | 64 |
| C8A | MESLGITSR | 497.26 | 533.30 | 35 | 17 | y5 | 64 |
| C8G | SLPVSDSVLSGFEQR | 540.95 | 723.34 | 35 | 18 | y6 | 49 |
| C8G | SLPVSDSVLSGFEQR | 540.95 | 636.31 | 35 | 18 | y5 | 49 |
| C9 | LSPIYNLVPVK | 621.88 | 832.49 | 35 | 22 | y7 | 50 |
| C9 | LSPIYNLVPVK | 621.88 | 521.82 | 35 | 22 | y9 | 50 |
| CFBb | DISEVVTPR | 508.27 | 787.43 | 35 | 18 | y7 | 63 |
| CFBb | DISEVVTPR | 508.27 | 571.36 | 35 | 18 | y5 | 63 |
| PON1 | IFFYDSENPPASEVLR | 628.64 | 771.44 | 35 | 21 | y7 | 52 |
| PON1 | IFFYDSENPPASEVLR | 628.64 | 434.75 | 35 | 21 | y8 | 52 |
| C3 | AEDLVGK | 366.20 | 660.36 | 35 | 12 | y6 | 43 |
| C3 | AEDLVGK | 366.20 | 531.31 | 35 | 12 | y5 | 43 |
| VTN | FEDGVLDPDYPR | 711.83 | 875.43 | 35 | 25 | y7 | 51 |
| VTN | FEDGVLDPDYPR | 711.83 | 647.31 | 35 | 25 | y5 | 51 |
| Total IgG | NQVSLTCLVK | 581.32 | 919.53 | 35 | 20 | y8 | 65 |
| Total IgG | NQVSLTCLVK | 581.32 | 820.46 | 35 | 20 | y7 | 65 |
| IgG1 | GPSVFPLAPSSK | 593.83 | 699.40 | 35 | 21 | y7 | 56 |
| IgG1 | GPSVFPLAPSSK | 593.83 | 418.23 | 35 | 21 | y4 | 56 |
| IgG2 | GLPAPIEK | 412.75 | 486.29 | 35 | 14 | y4 | 57 |
| IgG2 | GLPAPIEK | 412.75 | 327.69 | 35 | 14 | y6 | 57 |
| IgG3 | TPLGDTTHTCPR | 452.55 | 579.27 | 35 | 15 | y10 | 58 |
| IgG3 | TPLGDTTHTCPR | 452.55 | 522.73 | 35 | 15 | v9 | 58 |
| IgG4 | GLPSSIEK | 415.73 | 563.30 | 35 | 14 | y5 | 61 |
| IgG4 | GLPSSIEK | 415.73 | 330.68 | 35 | 14 | y6 | 61 |
| IgA1 | TPLTATLSK | 466.28 | 620.36 | 35 | 16 | y6 | 59 |
| IgA1 | TPLTATLSK | 466.28 | 415.75 | 35 | 16 | y8 | 59 |
| IgM | GFPSVLR | 388.23 | 474.30 | 35 | 13 | y4 | 60 |
| IgM | GFPSVLR | 388.23 | 286.18 | 35 | 13 | y5 | 60 |
| Kappa | VDNALQSGNSQESVTE | 712.66 | 893.42 | 35 | 24 | y8 | 66 |
| | QDSK | | | | | | |
| Kappa | VDNALQSGNSQESVTE | 712.66 | 707.32 | 35 | 24 | y6 | 66 |
| | QDSK | | | | | | |
| Lambda | YAASSYLSLTPEQWK | 872.43 | 988.51 | 35 | 31 | y8 | 67 |
| Lambda | YAASSYLSLTPEQWK | 872.43 | 687.35 | 35 | 31 | y5 | 67 |
| SARS-COV2 spike | GVYYPDK | 421.21 | 685.32 | 35 | 15 | y5 | 68 |
| SARS-COV2 spike | GVYYPDK | 421.21 | 359.19 | 35 | 15 | y3 | 68 |
| SARS-COV2 spike | FASVYAWNR | 557.28 | 808.41 | 35 | 20 | y6 | 69 |
| SARS-COV2 spike | FASVYAWNR | 557.28 | 709.34 | 35 | 20 | y5 | 69 |
| SARS-COV2 spike | QIAPGQTGK | 450.25 | 658.35 | 35 | 16 | y7 | 71 |
| SARS-COV2 spike | QIAPGQTGK | 450.25 | 587.31 | 35 | 16 | y6 | 71 |
| SARS-COV2 spike | VGGNYNYLYR | 609.80 | 1119.52 | 35 | 21 | y9 | 70 |
| SARS-COV2 spike | VGGNYNYLYR | 609.80 | 728.37 | 35 | 21 | y5 | 70 |
| C1QA | SLGFCDTTNK | 571.76 | 942.40 | 35 | 20 | y8 | 44 |
| C1QA | SLGFCDTTNK | 571.76 | 738.31 | 35 | 20 | y6 | 44 |
| C1QC | FQSVFTVTR | 542.79 | 809.45 | 35 | 19 | y7 | 45 |
| C1QC | FQSVFTVTR | 542.79 | 809.42 | 35 | 19 | b7 | 45 |
| C1QB | GNLCVNLMR | 538.77 | 792.39 | 35 | 19 | y6 | 46 |
| C1QB | GNLCVNLMR | 538.77 | 632.35 | 35 | 19 | y5 | 46 |

### REFERENCES

1. Reynolds, C. J.; Pade, C.; Gibbons, J. M.; Butler, D. K.; Otter, A. D.; Menacho, K.; Fontana, M.; Smit, A.; Sackville-West, J. E.; Cutino-Moguel, T.; Maini, M. K.; Chain, B.; Noursadeghi, M.; Network, U. K. C. I. C.; Brooks, T.; Semper, A.; Manisty, C.; Treibel, T. A.; Moon, J. C.; Investigators, U. K. C.; Valdes, A. M.; McKnight, A.; Altmann, D. M.; Boyton, R., Prior SARS-CoV-2 infection rescues B and T cell responses to variants after first vaccine dose. Science 2021.
2. Ferrante, A.; Beard, L. J.; Feldman, R. G., IgG subclass distribution of antibodies to bacterial and viral antigens. Pediatr Infect Dis J 1990, 9 (8 Suppl), S16-24.
3. Blue, C. E.; Spiller, O. B.; Blackbourn, D. J., The relevance of complement to virus biology. Virology 2004, 319 (2), 176-84.
4. Damelang, T.; Rogerson, S. J.; Kent, S. J.; Chung, A. W., Role of IgG3 in Infectious Diseases. Trends Immunol 2019, 40 (3), 197-211.
5. Lee, C. H.; Romain, G.; Yan, W.; Watanabe, M.; Charab, W.; Todorova, B.; Lee, J.; Triplett, K.; Donkor, M.; Lungu, O. I.; Lux, A.; Marshall, N.; Lindorfer, M. A.; Goff, O. R.; Balbino, B.; Kang, T. H.; Tanno, H.; Delidakis, G.; Alford, C.; Taylor, R. P.; Nimmerjahn, F.; Varadarajan, N.; Bruhns, P.; Zhang, Y. J.; Georgiou, G., IgG Fc domains that bind C1q but not effector Fcgamma receptors delineate the importance of complement-mediated effector functions. Nat Immunol 2017, 18 (8), 889-898.
6. Vidarsson, G.; Dekkers, G.; Rispens, T., IgG subclasses and allotypes: from structure to effector functions. Frontiers in immunology 2014, 5, 520.
7. Yates, N. L.; Liao, H. X.; Fong, Y.; deCamp, A.; Vandergrift, N. A.; Williams, W. T.; Alam, S. M.; Ferrari, G.; Yang, Z. Y.; Seaton, K. E.; Berman, P. W.; Alpert, M. D.; Evans, D. T.; O'Connell, R. J.; Francis, D.; Sinangil, F.; Lee, C.; Nitayaphan, S.; Rerks-Ngarm, S.; Kaewkungwal, J.; Pitisuttithum, P.; Tartaglia, J.; Pinter, A.; Zolla-Pazner, S.; Gilbert, P. B.; Nabel, G. J.; Michael, N. L.; Kim, J. H.; Montefiori, D. C.; Haynes, B. F.; Tomaras, G. D., Vaccine-induced Env V1-V2 IgG3 correlates with lower HIV-1 infection risk and declines soon after vaccination. Science translational medicine 2014, 6 (228), 228ra39.
8. Stapleton, N. M.; Andersen, J. T.; Stemerding, A. M.; Bjarnarson, S. P.; Verheul, R. C.; Gerritsen, J.; Zhao, Y.; Kleijer, M.; Sandlie, I.; de Haas, M.; Jonsdottir, I.; van der Schoot, C. E.; Vidarsson, G., Competition for FcRn-mediated transport gives rise to short half-life of human IgG3 and offers therapeutic potential. Nat Commun 2011, 2, 599.
9. Yu, H. Q.; Sun, B. Q.; Fang, Z. F.; Zhao, J. C.; Liu, X. Y.; Li, Y. M.; Sun, X. Z.; Liang, H. F.; Zhong, B.; Huang, Z. F.; Zheng, P. Y.; Tian, L. F.; Qu, H. Q.; Liu, D. C.; Wang, E. Y.; Xiao, X. J.; Li, S. Y.; Ye, F.; Guan, L.; Hu, D. S.; Hakonarson, H.; Liu, Z. G.; Zhong, N. S., Distinct features of SARS-CoV-2-specific IgA response in COVID-19 patients. Eur Respir J 2020.
10. Sterlin, D.; Mathian, A.; Miyara, M.; Mohr, A.; Anna, F.; Claer, L.; Quentric, P.; Fadlallah, J.; Devilliers, H.; Ghillani, P.; Gunn, C.; Hockett, R.; Mudumba, S.; Guihot, A.; Luyt, C. E.; Mayaux, J.; Beurton, A.; Fourati, S.; Bruel, T.; Schwartz, O.; Lacorte, J. M.; Yssel, H.; Parizot, C.; Dorgham, K.; Charneau, P.; Amoura, Z.; Gorochov, G., IgA dominates the early neutralizing antibody response to SARS-CoV-2. Science translational medicine 2021, 13 (577).
11. Liew, F. Y.; Russell, S. M.; Appleyard, G.; Brand, C. M.; Beale, J., Cross-protection in mice infected with influenza A virus by the respiratory route is correlated with local IgA antibody rather than serum antibody or cytotoxic T cell reactivity. Eur J Immunol 1984, 14 (4), 350-6.
12. Asahi-Ozaki, Y.; Yoshikawa, T.; Iwakura, Y.; Suzuki, Y.; Tamura, S.; Kurata, T.; Sata, T., Secretory IgA antibodies provide cross-protection against infection with different strains of influenza B virus. J Med Virol 2004, 74 (2), 328-35.
13. Vasantha, S.; Coelingh, K. L.; Murphy, B. R.; Dourmashkin, R. R.; Hammer, C. H.; Frank, M. M.; Fries, L. F., Interactions of a nonneutralizing IgM antibody and complement in parainfluenza virus neutralization. Virology 1988, 167 (2), 433-41.
14. Jayasekera, J. P.; Moseman, E. A.; Carroll, M. C., Natural antibody and complement mediate neutralization of influenza virus in the absence of prior immunity. J Virol 2007, 81 (7), 3487-94.
15. Public Health England, COVID-19: laboratory evaluations of serological assays, gov.uk, 16 Mar. 2021.
16. Bliss, E.; Heywood, W. E.; Benatti, M.; Sebire, N. J.; Mills, K., An optimised method for the proteomic profiling of full thickness human skin. Biol Proced Online 2016, 18, 15.

### REFERENCES (2)

1. Public Health England, COVID-19: laboratory evaluations of serological assays, gov.uk, 16 March 2021.
2. Abbasi, J. (2021). The Flawed Science of Antibody Testing for SARS-CoV-2 Immunity. JAMA 326, 1781-1782.
3. Amanat, F., Stadlbauer, D., Strohmeier, S., Nguyen, T. H. O., Chromikova, V., McMahon, M., Jiang, K., Asthagiri Arunkumar, G., Jurczyszak, D., Polanco, J., et al. (2020). A serological assay to detect SARS-CoV-2 seroconversion in humans. medRxiv.
4. Anichini, G., Terrosi, C., Gandolfo, C., Gori Savellini, G., Fabrizi, S., Miceli, G. B., and Cusi, M. G. (2021). SARS-CoV-2 Antibody Response in Persons with Past Natural Infection. The New England journal of medicine 385, 90-92.
5. Dejnirattisai, W., Shaw, R. H., Supasa, P., Liu, C., Stuart, A. S., Pollard, A. J., Liu, X., Lambe, T., Crook, D., Stuart, D. I., et al. (2022). Reduced neutralisation of SARS-CoV-2 omicron B.1.1.529 variant by post-immunisation serum. Lancet 399, 234-236.
6. Della-Torre, E., Lanzillotta, M., Strollo, M., Ramirez, G. A., Dagna, L., Tresoldi, M., and group, C.O.-B.s. (2021). Serum IgG4 level predicts COVID-19 related mortality. Eur J Intern Med 93, 107-109.
7. Dogan, M., Kozhaya, L., Placek, L., Gunter, C., Yigit, M., Hardy, R., Plassmeyer, M., Coatney, P., Lillard, K., Bukhari, Z., et al. (2021). SARS-CoV-2 specific antibody and neutralization assays reveal the wide range of the humoral immune response to virus. Commun Biol 4, 129.
8. Ebinger, J. E., Fert-Bober, J., Printsev, I., Wu, M., Sun, N., Prostko, J. C., Frias, E. C., Stewart, J. L., Van Eyk, J. E., Braun, J. G., et al. (2021). Antibody responses to the BNT162b2 mRNA vaccine in individuals previously infected with SARS-CoV-2. Nature medicine 27, 981-984.
9. Ferrante, A., Beard, L. J., and Feldman, R. G. (1990). IgG subclass distribution of antibodies to bacterial and viral antigens. Pediatr Infect Dis J 9, S16-24.
10. Gaebler, C., Wang, Z., Lorenzi, J. C. C., Muecksch, F., Finkin, S., Tokuyama, M., Cho, A., Jankovic, M., Schaefer-Babajew, D., Oliveira, T. Y., et al. (2021). Evolution of antibody immunity to SARS-CoV-2. Nature 591, 639-644.
11. Jarlhelt, I., Nielsen, S. K., Jahn, C. X. H., Hansen, C. B., Perez-Alos, L., Rosbjerg, A., Bayarri-Olmos, R., Skjoedt, M. O., and Garred, P. (2021). SARS-CoV-2 Antibodies Mediate Complement and Cellular Driven Inflammation. Frontiers in immunology 12, 767981.
12. Jayasekera, J. P., Moseman, E. A., and Carroll, M. C. (2007). Natural antibody and complement mediate neutralization of influenza virus in the absence of prior immunity. J Virol 81, 3487-3494.
13. Ju, B., Zhang, Q., Ge, J., Wang, R., Sun, J., Ge, X., Yu, J., Shan, S., Zhou, B., Song, S., et al. (2020). Human neutralizing antibodies elicited by SARS-CoV-2 infection. Nature 584, 115-119.
14. Kim, P., Gordon, S. M., Sheehan, M. M., and Rothberg, M. B. (2021). Duration of SARS-CoV-2 Natural Immunity and Protection against the Delta Variant: A Retrospective Cohort Study. Clin Infect Dis.
15. Kunnakkadan, U., Nag, J., Kumar, N. A., Mukesh, R. K., Suma, S. M., and Johnson, J. B. (2019). Complement-Mediated Neutralization of a Potent Neurotropic Human Pathogen, Chandipura Virus, Is Dependent on C1q. J Virol 93.
16. Manisty, C., Otter, A. D., Treibel, T. A., McKnight, A., Altmann, D. M., Brooks, T., Noursadeghi, M., Boyton, R. J., Semper, A., and Moon, J. C. (2021a). Antibody response to first BNT162b2 dose in previously SARS-CoV-2-infected individuals. Lancet 397, 1057-1058.
17. Manisty, C., Treibel, T. A., Jensen, M., Semper, A., Joy, G., Gupta, R. K., Cutino-Moguel, T., Andiapen, M., Jones, J., Taylor, S., et al. (2021b). Time series analysis and mechanistic modelling of heterogeneity and sero-reversion in antibody responses to mild SARSCoV-2 infection. EBioMedicine 65, 103259.
18. Mehlhop, E., Nelson, S., Jost, C. A., Gorlatov, S., Johnson, S., Fremont, D. H., Diamond, M. S., and Pierson, T. C. (2009). Complement protein C1q reduces the stoichiometric threshold for antibody-mediated neutralization of West Nile virus. Cell Host Microbe 6, 381-391.
19. Nie, J., Li, Q., Wu, J., Zhao, C., Hao, H., Liu, H., Zhang, L., Nie, L., Qin, H., Wang, M., et al. (2020). Quantification of SARS-CoV-2 neutralizing antibody by a pseudotyped virus-based assay. Nat Protoc 15, 3699-3715.
20. Pastorino, B., Touret, F., Gilles, M., de Lamballerie, X., and Charrel, R. N. (2020).

Heat Inactivation of Different Types of SARS-CoV-2 Samples: What Protocols for Biosafety, Molecular Detection and Serological Diagnostics? Viruses 12.
21. Patil, H. P., Rane, P. S., Shrivastava, S., Palkar, S., Lalwani, S., Mishra, A. C., and Arankalle, V. A. (2021). Antibody (IgA, IgG, and IgG Subtype) Responses to SARS-CoV-2 in Severe and Nonsevere COVID-19 Patients. Viral Immunol 34, 201-209.
22. Perez-Toledo, M., Faustini, S. E., Jossi, S. E., Shields, A. M., Marcial-Juarez, E., Kanthimathinathan, H. K., Allen, J. D., Watanabe, Y., Goodall, M., Willcox, B. E., et al. (2021). SARS-CoV-2-specific IgG1/IgG3 but not IgM in children with Pediatric Inflammatory Multi-System Syndrome. Pediatr Allergy Immunol 32, 1125-1129.
23. Reynolds, C. J., Gibbons, J. M., Pade, C., Lin, K. M., Sandoval, D. M., Pieper, F., Butler, D. K., Liu, S., Otter, A. D., Joy, G., et al. (2021a). Heterologous infection and vaccination shapes immunity against SARS-CoV-2 variants. Science, eabm0811.
24. Reynolds, C. J., Pade, C., Gibbons, J. M., Butler, D. K., Otter, A. D., Menacho, K., Fontana, M., Smit, A., Sackville-West, J. E., Cutino-Moguel, T., et al. (2021b). Prior SARS-CoV-2 infection rescues B and T cell responses to variants after first vaccine dose. Science.
25. Reynolds, C. J., Swadling, L., Gibbons, J. M., Pade, C., Jensen, M. P., Diniz, M. O., Schmidt, N. M., Butler, D. K., Amin, O. E., Bailey, S. N. L., et al. (2020). Discordant neutralizing antibody and T cell responses in asymptomatic and mild SARS-CoV-2 infection. Sci Immunol 5.
26. Reynolds, (2022). Reynolds C J, Pade C, Gibbons J M, Otter A D, Lir K M, Munioz Sandoval D. Pieper F P, Butler D K, Liu S, Joy G. Forooghi N, Treibel T A, Manisty C, Moon J C; COVIDsortiurm Investigators‡; COVIDsortium Immune Correlates Network‡; Semper A, Brooks T, McKnight Á, Altmann D M, Boyton R J Immune boosting by B.1.1.529 (Omicron) depends on previous SARS-CoV-2 exposure. Science. 2022 Jun. 14:eabq1841. doi: 10.1126/science.abq1841.
27. Saxena, S. K., Kumar, S., Ansari, S., Paweska, J. T., Maurya, V. K., Tripathi, A. K., and Abdel-Moneim, A. S. (2022). Characterization of the novel SARS-CoV-2 Omicron (B.1.1.529) variant of concern and its global perspective. J Med Virol 94, 1738-1744.
28. Schiela, B., Bernklau, S., Malekshahi, Z., Deutschmann, D., Koske, I., Banki, Z., Thielens, N. M., Wurzner, R., Speth, C., Weiss, G., et al. (2018). Active Human Complement Reduces the Zika Virus Load via Formation of the Membrane-Attack Complex. Frontiers in immunology 9, 2177.
29. Smit, N. P. M., Ruhaak, L. R., Romijn, F., Pieterse, M. M., van der Burgt, Y. E. M., and Cobbaert, C. M. (2021). The Time Has Come for Quantitative Protein Mass Spectrometry Tests That Target Unmet Clinical Needs. J Am Soc Mass Spectrom 32, 636-647.
30. Treibel, T. A., Manisty, C., Burton, M., McKnight, A., Lambourne, J., Augusto, J. B., Couto-Parada, X., Cutino-Moguel, T., Noursadeghi, M., and Moon, J. C. (2020). COVID-19: PCR screening of asymptomatic health-care workers at London hospital. Lancet 395, 1608-1610.
31. van der Neut Kolfschoten, M., Schuurman, J., Losen, M., Bleeker, W. K., Martinez-Martinez, P., Vermeulen, E., den Bleker, T. H., Wiegman, L., Vink, T., Aarden, L. A., et al. (2007). Anti-inflammatory activity of human IgG4 antibodies by dynamic Fab arm exchange. Science 317, 1554-1557.
32. Vasantha, S., Coelingh, K. L., Murphy, B. R., Dourmashkin, R. R., Hammer, C. H., Frank, M. M., and Fries, L. F. (1988). Interactions of a nonneutralizing IgM antibody and complement in parainfluenza virus neutralization. Virology 167, 433-441.
33. Wang, L., and Cheng, G. (2022). Sequence analysis of the emerging SARS-CoV-2 variant Omicron in South Africa. J Med Virol 94, 1728-1733.
34. Yang, T. J., Yu, P. Y., Chang, Y. C., Liang, K. H., Tso, H. C., Ho, M. R., Chen, W. Y., Lin, H. T., Wu, H. C., and Hsu, S. D. (2021). Effect of SARS-CoV-2 B.1.1.7 mutations on spike protein structure and function. Nat Struct Mol Biol 28, 731-739.
35. Yu, H. Q., Sun, B. Q., Fang, Z. F., Zhao, J. C., Liu, X. Y., Li, Y. M., Sun, X. Z., Liang, H. F., Zhong, B., Huang, Z. F., et al. (2020). Distinct features of SARS-CoV-2-specific IgA response in COVID-19 patients. Eur Respir J.
36. Yu, X., Wei, D., Xu, W., Li, Y., Li, X., Zhang, X., Qu, J., Yang, Z., and Chen, E. (2022). Reduced sensitivity of SARS-CoV-2 Omicron variant to antibody neutralization elicited by booster vaccination. Cell Discov 8, 4.
37. Zhou, D., Dejnirattisai, W., Supasa, P., Liu, C., Mentzer, A. J., Ginn, H. M., Zhao, Y., Duyvesteyn, H. M. E., Tuekprakhon, A., Nutalai, R., et al. (2021). Evidence of escape of SARS-CoV-2 variant B.1.351 from natural and vaccine-induced sera. Cell 184, 2348-2361 e2346.

1. A method of identifying and quantifying one or more binding molecules in a biological fluid of an individual, wherein the binding molecules bind to a bait molecule, the method comprising:
a. providing a sample comprising biological fluid which has been taken from the individual;
b. capturing the one or more binding molecules by contacting the sample with a plurality of the bait molecules to form binding complexes comprising the bait molecules and the one or more binding molecules;
c. isolating the binding complexes; and
d. identifying and quantifying the one or more binding molecules in the biological fluid by analysing the captured binding molecules.

2. A method according to claim 1, wherein following the step of isolating the binding complexes the complexes are subjected to a digestion step to produce digestion products, and wherein the one or more binding molecules are identified and quantified by analysing the digestion products.

3. A method according to claim 2, wherein the one or more binding molecules are proteins, wherein following isolation the complexes are subjected to proteolytic digestion to produce proteolytic digestion products, and wherein the one or more binding molecules are identified and quantified by analysing the proteolytic digestion products.

4. A method according to any one of the preceding claims, wherein the step of analysing the captured binding molecules is performed by mass spectrometry, preferably by liquid chromatography-tandem mass spectrometry (LC-MS/MS).

5. A method according to any one of claims 1-4, wherein bait molecules have been bound to a surface prior to contact with the sample, preferably wherein the plurality of bait molecules have been bound to the surface of a microplate.

6. A method according to any one of claims 1-4, wherein bait molecules are bound to a surface during the process of isolating the binding complexes, preferably wherein the plurality of bait molecules are bound to the surface of a microparticle.

7. A method according to any one of the preceding claims, wherein the bait molecule comprises a pathogen polypeptide; an autoantigen polypeptide; a neoantigen polypeptide or a polypeptide of a gene therapy or vaccine delivery vector, such as a polypeptide of a viral vector e.g. an adenoviral vector, preferably AAV.

8. A method according to claim 7, wherein the pathogen polypeptide is a viral polypeptide.

9. A method according to claim 8, wherein the viral polypeptide is a coronavirus polypeptide, preferably wherein the coronavirus is severe acute respiratory syndrome coronavirus 2 (SARS-CoV-2).

10. A method according to claim 9, wherein the viral polypeptide comprises:
a. a full-length coronavirus spike protein, or a subunit or a fragment thereof,
b. a full-length coronavirus envelope protein or a fragment thereof,
c. a full-length coronavirus membrane protein or a fragment thereof, or
d. a full-length coronavirus nucleocapsid protein or a fragment thereof; and
preferably wherein the coronavirus is severe acute respiratory syndrome coronavirus 2 (SARS-CoV-2).

11. A method according to claim 10, wherein the viral polypeptide comprises:
a. an S1 subunit of a SARS-CoV-2 coronavirus spike protein or a fragment thereof, preferably wherein the S1 subunit comprises or consists of the amino acid sequence
(SEQ ID NO: 1)
QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGTKCTLKSFTVE
KGIYQTSNFRVQPTESIVRFPNITNLCPFGEVENATRFASVYAWNRKRIS
NCVADYSVLYNSASFSTFKCYGVSPTKLNDLCFTNVYADSFVIRGDEVRQ
IAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYR
VVVLSFELLHAPATVCGPKKSTNLVKNKCVNFNFNGLTGTGVLTESNKKF
LPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNS
YECDIPIGAGICASYQTQTNSPRRAR;
b. an S2 subunit of a SARS-CoV-2 coronavirus spike protein or a fragment thereof, preferably wherein the S2 subunit comprises or consists of the amino acid sequence
(SEQ ID NO: 2)
SVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFIK
QYGDCLGDIAARDLICAQKFNGLTVLPPLLTDEMIAQYTSALLAGTITSG
WTFGAGAALQIPFAMQMAYRENGIGVTQNVLYENQKLIANQFNSAIGKIQ
DSLSSTASALGKLQDVVNQNAQALNTLVKQLSSNFGAISSVLNDILSRLD
KVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDR
LNEVAKNLNESLIDLQELGKYEQYIKWPWYIWLGFIAGLIAIVMVTIMLC
CMTSCCSCLKGCCSCGSCCKFDEDDSEPVLKGVKLHYT;
c. an angiotensin-converting enzyme 2 (ACE2) receptor binding domain (RBD) of an S1 subunit of SARS-CoV-2 coronavirus spike protein or a fragment thereof, preferably wherein the RBD of the S1 subunit comprises or consist of the amino acid sequence
(SEQ ID NO: 3)
RVQPTESIVRFPNITNLCPFGEVENATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGENCYFPLQSYGFQPTNGVGYQPYRVVVLSFELL
HAPATVCGPKKSTNLVKNKCVNF.

12. A method according to claim 10 or 11, wherein the plurality of bait molecules comprises a subunit of a coronavirus protein, or a fragment of a coronavirus protein, and wherein the subunit or fragment is an immunogenic subunit or fragment.

13. A method according to any one of claims 1-6, wherein the bait molecule comprises a virion; preferably a virion that is not capable of replication, such as a pseudovirus.

14. A method according to claim 14, wherein the virion is a coronavirus virion, preferably a severe acute respiratory syndrome coronavirus 2 (SARS-CoV-2) virion.

15. A method according to any one of claims 8 to 14, wherein (i) the viral polypeptide, (ii) the full-length coronavirus spike protein, subunit, RBD or any fragment thereof; (iii) the full-length coronavirus envelope protein or fragment thereof; (iv) the full-length coronavirus membrane protein or fragment thereof; (v) the full-length coronavirus nucleocapsid protein or fragment thereof; or (vi) the coronavirus virion is from:
a. wild-type SARS-CoV-2; or
b. a variant of wild-type SARS-CoV-2, wherein the amino acid sequence of the variant differs from the amino acid sequence of wild-type by one or more amino acid mutations, optionally wherein one or more amino acid mutations are in an envelope protein or wherein one or more amino acid mutations are in a nucleocapsid protein, preferably wherein one or more amino acid mutations are in the spike protein.

16. A method according to claim 15, wherein (i) the viral polypeptide, (ii) the full-length coronavirus spike protein, subunit, RBD or any fragment thereof; (iii) the full-length coronavirus envelope protein or fragment thereof; (iv) the full-length coronavirus membrane protein or fragment thereof; (v) the full-length coronavirus nucleocapsid protein or fragment thereof; or (vi) the coronavirus virion is from:
a. SARS-CoV-2 wild-type;
b. SARS-CoV-2 B.1.1.7 variant (alpha variant);
c. SARS-CoV-2 B.1.351 variant (beta variant);
d. SARS-CoV-2 P.1 variant (gamma variant);
e. SARS-CoV-2 B.1.617.2 variant (delta variant);
f. SARS-CoV-2 B.1.427/B.1.429 variant (epsilon variant);
g. SARS-CoV-2 P.2 variant (zeta variant);
h. SARS-CoV-2 B.1.525 variant (eta variant);
i. SARS-CoV-2 P.3 variant (theta variant);
j. SARS-CoV-2 B.1.526 variant (Iota variant);
k. SARS-CoV-2 B.1.617.1 variant (kappa variant);
l. SARS-CoV-2 C.37 variant (lambda variant); or
m. SARS-CoV-2 B.1.1.529 variant (omicron variant).

17. A method according to any one of claims 7-16, wherein the one or more binding molecules which are identified and quantified are antibodies of IgG isotype, and wherein the antibodies are quantified according to concentration in the biological fluid of the individual.

18. A method according to claim 17, further comprising determining whether or not the IgG antibody concentration in the biological fluid of the individual is statistically significantly increased compared to the IgG antibody concentration in a control, wherein the control is the average IgG antibody concentration in a population of individuals which have not been vaccinated against SARS-CoV-2 and which have not been infected with SARS-CoV-2.

19. A method according to claim 17, further comprising determining whether or not the IgG antibody concentration in the biological fluid of the individual is statistically significantly increased compared to the IgG antibody concentration in a control, wherein the control is the average IgG antibody concentration in a population of individuals which have been vaccinated against SARS-CoV-2 and which have not been infected with SARS-CoV-2.

20. A method according to any one of claims 17 to 19, wherein the one or more binding molecules which are identified and quantified are antibodies of isotype IgG1, IgG2, IgG3 and/or IgG4, preferably IgG1.

21. A method according to any one of claims 7-16, wherein the one or more binding molecules which are identified and quantified are antibodies of IgA isotype, preferably IgA1, and wherein the antibodies are quantified according to concentration in the biological fluid of the individual.

22. A method according to claim 21, further comprising determining whether or not the IgA antibody concentration in the biological fluid of the individual is statistically significantly increased compared to the IgA antibody concentration in a control, wherein the control is the average IgA antibody concentration in a population of individuals which have not been vaccinated against SARS-CoV-2 and which have not been infected with SARS-CoV-2.

23. A method according to claim 21, further comprising determining whether or not the IgA antibody concentration in the biological fluid of the individual is statistically significantly increased compared to the IgA antibody concentration in a control, wherein the control is the average IgA antibody concentration in a population of individuals which have been vaccinated against SARS-CoV-2 and which have not been infected with SARS-CoV-2.

24. A method according to any one of claims 7-16, wherein the one or more binding molecules which are identified and quantified are antibodies of IgM isotype and wherein the antibodies are quantified according to concentration in the biological fluid of the individual.

25. A method according to claim 24, further comprising determining whether or not the IgM antibody concentration in the biological fluid of the individual is statistically significantly increased compared to the IgM antibody concentration in a control, wherein the control is the average IgM antibody concentration in a population of individuals which have not been vaccinated against SARS-CoV-2 and which have not been infected with SARS-CoV-2.

26. A method according to claim 24, further comprising determining whether or not the IgM antibody concentration in the biological fluid of the individual is statistically significantly increased compared to the IgM antibody concentration in a control, wherein the control is the average IgM antibody concentration in a population of individuals which have been vaccinated against SARS-CoV-2 and which have not been infected with SARS-CoV-2.

27. A method according to any one of claims 17 to 26, wherein quantification by mass spectrometry of antibodies of the defined isotype is performed by detecting a conserved peptide sequence common to all species of that defined isotype, and wherein the peptide sequence does not occur in the antibody variable region.

28. A method according to any one of claims 7-16, wherein the binding molecules which are identified and quantified are complement C4b-binding protein (C4BP) and wherein the proteins are quantified according to their concentration in the biological fluid of the individual.

29. A method according to claim 27, further comprising determining whether or not the C4BP protein concentration in the biological fluid of the individual is statistically significantly increased compared to the C4BP protein concentration in a control, wherein the control is the average C4BP protein concentration in a population of individuals which have not been vaccinated against SARS-CoV-2 and which have not been infected with SARS-CoV-2.

30. A method according to claim 27, further comprising determining whether or not the C4BP protein concentration in the biological fluid of the individual is statistically significantly increased compared to the C4BP protein concentration in a control, wherein the control is the average C4BP protein concentration in a population of individuals which have been vaccinated against SARS-CoV-2 and which have not been infected with SARS-CoV-2.

31. A method according to any one of claims 7-16, wherein the binding molecules which are identified and quantified are complement component 4B (C4B) and wherein the proteins are quantified according to their concentration in the biological fluid of the individual.

32. A method according to claim 30, further comprising determining whether or not the C4B protein concentration in the biological fluid of the individual is statistically significantly increased compared to the C4B protein concentration in a control, wherein the control is the average C4B protein concentration in a population of individuals which have not been vaccinated against SARS-CoV-2 and which have not been infected with SARS-CoV-2.

33. A method according to claim 30, further comprising determining whether or not the C4B protein concentration in the biological fluid of the individual is statistically significantly increased compared to the C4B protein concentration in a control, wherein the control is the average C4B protein concentration in a population of individuals which have been vaccinated against SARS-CoV-2 and which have not been infected with SARS-CoV-2.

34. A method according to any one of claims 7-16, wherein the binding molecules which are identified and quantified are complement component 9 (C9) and wherein the proteins are quantified according to their concentration in the biological fluid of the individual.

35. A method according to claim 34, further comprising determining whether or not the C9 protein concentration in the biological fluid of the individual is statistically significantly increased compared to the C9 protein concentration in a control, wherein the control is the average C9 protein concentration in a population of individuals which have not been vaccinated against SARS-CoV-2 and which have not been infected with SARS-CoV-2.

36. A method according to claim 34, further comprising determining whether or not the C9 protein concentration in the biological fluid of the individual is statistically significantly increased compared to the C9 protein concentration in a control, wherein the control is the average C9 protein concentration in a population of individuals which have been vaccinated against SARS-CoV-2 and which have not been infected with SARS-CoV-2.

37. A method according to any one of claims 7-16, wherein the binding molecules which are identified and quantified are complement C1q and wherein the proteins are quantified according to their concentration in the biological fluid of the individual.

38. A method according to claim 37, further comprising determining whether or not the C1q protein concentration in the biological fluid of the individual is statistically significantly increased compared to the C1q protein concentration in a control, wherein the control is the average C1q protein concentration in a population of individuals which have not been vaccinated against SARS-CoV-2 and which have not been infected with SARS-CoV-2.

39. A method according to claim 37, further comprising determining whether or not the C1q protein concentration in the biological fluid of the individual is statistically significantly increased compared to the C1q protein concentration in a control, wherein the control is the average C1q protein concentration in a population of individuals which have been vaccinated against SARS-CoV-2 and which have not been infected with SARS-CoV-2.

40. A method according to any one of claims 18, 20, 22, 25, 29, 32, 35 or 38, wherein when the antibody concentration or protein concentration is statistically significantly increased compared to the antibody concentration or protein concentration in the control, the individual is classified as having produced antibodies to SARS-CoV-2 or produced proteins following exposure to SARS-CoV-2 antigens, and wherein the individual is thereby classified as having mounted an immune response to SARS-CoV-2.

41. A method according to any one of claims 18, 20, 22, 25, 29, 32, 35 or 38, wherein when the antibody concentration or protein concentration is not statistically significantly increased compared to the antibody concentration or protein concentration in the control, the individual is classified as requiring a prime dose of a vaccine against SARS-CoV-2 or a booster dose of a vaccine against SARS-CoV-2.

42. A method according to any one of claims 19, 20, 23, 26, 30, 33, 36, or 39, wherein when the antibody concentration or protein concentration is statistically significantly increased compared to the antibody concentration or protein concentration in the control, the individual is predicted to have been infected with SARS-CoV-2.

43. A method according to claim 17, further comprising determining in the biological fluid of the individual an IgG antibody concentration which causes inhibition of the biological activity of SARS-CoV-2 and wherein the individual is thereby classified as having generated a neutralising antibody response to SARS-CoV-2, preferably wherein the biological activity of SARS-CoV-2 is the ability of SARS-CoV-2 to infect a human cell expressing the ACE2 receptor, preferably wherein the one or more binding molecules which are identified and quantified are antibodies of isotype IgG1, IgG2, IgG3 and/or IgG4.

44. A method according to any one of claims 18 to 43, wherein the method exhibits a sensitivity of 98.8% and a specificity of 100%.

45. A method according to any one of claims 1-6, wherein the plurality of bait molecules comprises a gene therapy or vaccine delivery vector, such as a viral vector, e.g. an adenoviral vector, preferably AAV; preferably wherein the gene therapy or vaccine delivery vector is not capable of replication.

46. A method according to claim 45, wherein the plurality of bait molecules comprises a vaccine viral delivery vector.

47. A method according to claim 46, wherein the plurality of bait molecules comprises a vaccine viral delivery vector selected from the group consisting of:
a. a replication-deficient adenoviral vector;
b. a replication defective adenoviral vector of ChAdOx1;
c. a replication defective adenoviral vector of serotype 5;
d. a replication defective adenoviral vector of serotype 26; and
e. replication defective adenoviral vectors of serotypes 5 and 26.

48. A method according to any one of the preceding claims, wherein the biological fluid is serum, plasma, whole blood, saliva, sputum, mucus or nasopharyngeal fluid, such as nasopharyngeal mucus.

49. A method according to claim 48, wherein the sample consists of biological fluid which has been taken from the individual and wherein the biological fluid in the sample has not been diluted.

50. A method according to claim 48, wherein the sample comprising biological fluid comprises blood obtained from a dried blood spot.

51. A method according to claim 48, wherein the sample comprises nasopharyngeal fluid, such as nasopharyngeal mucus, obtained from a nasopharyngeal swab.

52. A method according to any one of claims 49 to 51, wherein the sample comprising biological fluid comprises saliva obtained from a home test kit.

53. A kit comprising:
a. one or more vials each comprising a plurality of identical isolated bait molecules for use in capturing one or more binding molecules from a sample comprising biological fluid which has been taken from an individual;
b. at least one microplate and/or one or more vials each comprising microparticles for use in isolating binding complexes between the bait molecules and the one or more binding molecules;
c. one or more vials each comprising a plurality of identical molecules for use as a reagent blank, optionally wherein at least one vial comprises molecules of bovine serum albumin or casein, preferably wherein at least one vial comprises molecules of myoglobin;
d. optionally one or more vials each comprising a dilution buffer for use in diluting biological fluid which has been taken from an individual, such as phosphate-buffered saline (PBS);
e. optionally or more vials comprising a wash buffer for use in washing binding complexes between the bait molecules and the one or more binding molecules, such as phosphate-buffered saline (PBS);
f. one or more vials comprising a surfactant for use in solubilising proteins;
g. one or more vials comprising a digestion buffer comprising an enzyme capable of cleaving proteins, preferably wherein the enzyme is a protease; and
h. one or more vials comprising a plurality of identical molecules for use as an internal calibration standard when performing quantification of a protein by mass spectrometry, preferably wherein the molecules are peptides.

54. A kit according to claim 53, wherein the one or more vials each comprising a plurality of identical isolated bait molecules comprises a pathogen polypeptide; an autoantigen polypeptide; a neoantigen polypeptide or a polypeptide of a gene therapy or vaccine delivery vector, such as a polypeptide of a viral vector e.g. an adenoviral vector, preferably AAV.

55. A kit according to claim 54, wherein the one or more vials each comprising a plurality of identical isolated bait molecules comprises a pathogen polypeptide, an autoantigen polypeptide, a neoantigen polypeptide, a polypeptide of a gene therapy or vaccine delivery vector, a virion, a gene therapy or vaccine delivery vector, or a viral polypeptide, subunit or any fragment thereof as defined in any one of claims 7 to 17.

56. A kit according to any one of claims 53 to 55, wherein the one or more vials comprising a surfactant for use in solubilising proteins comprises a surfactant which is:
a. sodium 3-[(2-methyl-2-undecyl-1,3-dioxolan-4-yl)methoxy]-1-propanesulfonate,
b. 3-[N,N-Dimethyl(3-myristoylaminopropyl)ammonio]propanesulfonate (amidosulfobetaine-14) or
c. 3, 12-α-Dihydroxy-5β-cholan-24-oic acid monosodium salt (sodium deoxycholate).

57. A kit according to any one of claims 53 to 56, wherein the one or more vials comprising a digestion buffer comprising an enzyme capable of cleaving proteins comprises a protease which is:
a. trypsin
b. chymotrypsin,
c. LysC,
d. LysN,
e. AspN,
f. GluC, or
g. ArgC.

58. A kit according to any one of claims 53 to 57, wherein the one or more vials comprising a plurality of identical molecules for use as an internal calibration standard comprises a plurality of identical heavy labelled peptides, preferably wherein the peptides are labelled using ¹³C₆¹⁵N₂-lysine and ¹³C₆¹⁵N₄-arginine.

59. A kit according to claim 58, wherein the plurality of identical heavy labelled peptides have an amino acid sequence which is identical to an amino sequence of a pathogen polypeptide, an autoantigen polypeptide, a neoantigen polypeptide, a polypeptide of a gene therapy or vaccine delivery vector, a virion, a gene therapy or vaccine delivery vector, or a viral polypeptide, subunit or any fragment thereof as defined in any one of claims 7 to 16.

60. A kit according to any one of claims 53 to 59, further comprising one or more vials each comprising a plurality of identical monoclonal antibody species, wherein antibodies of a vial bind to an amino acid sequence which is common between two, more or all of:
a. wild-type SARS-CoV-2;
b. a variant of wild-type SARS-CoV-2, wherein the amino acid sequence of the variant differs from the amino acid sequence of wild-type by one or more amino acid mutations, optionally wherein one or more amino acid mutations are in an envelope protein or wherein one or more amino acid mutations are in a nucleocapsid protein, preferably wherein one or more amino acid mutations are in the spike protein;
c. SARS-CoV-2 B.1.1.7 variant (alpha variant);
d. SARS-CoV-2 B.1.351 variant (beta variant);
e. SARS-CoV-2 P.1 variant (gamma variant);
f. SARS-CoV-2 B.1.617.2 variant (delta variant);
g. SARS-CoV-2 B.1.427/B.1.429 variant (epsilon variant);
h. SARS-CoV-2 P.2 variant (zeta variant);
i. SARS-CoV-2 B.1.525 variant (eta variant);
j. SARS-CoV-2 P.3 variant (theta variant);
k. SARS-CoV-2 B.1.526 variant (Iota variant);
l. SARS-CoV-2 B.1.617.1 variant (kappa variant);
m. SARS-CoV-2 C.37 variant (lambda variant);
n. SARS-CoV-2 B.1.1.529 variant (omicron variant); and
wherein the amino acid sequence is a sequence of (i) the full-length coronavirus spike protein, subunit, RBD or any fragment thereof; (ii) the full-length coronavirus envelope protein or a fragment thereof; (iii) the full-length coronavirus membrane protein or a fragment thereof; or (iv) the full-length coronavirus nucleocapsid protein or a fragment thereof.


SEQUENCE LISTING (71 sequences)

SEQ ID NO: 1 - protein, 672 aa, SARS-CoV-2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 2 - protein, 588 aa, SARS-CoV-2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 223 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 4 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 13 aa, SARS-CoV-2
MFVFLVLLPLVSS

SEQ ID NO: 6 - protein, 292 aa, SARS-CoV-2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKS

SEQ ID NO: 7 - protein, 19 aa, SARS-CoV-2
IYKTPPIKDFGGFNFSQIL

SEQ ID NO: 8 - protein, 73 aa, SARS-CoV-2
TQNVLYENQKLIANQFNSAIGKIQDSLSSTASALGKLQDVVNQNAQALNTLVKQLSSNFGAISSVLNDILSRL

SEQ ID NO: 9 - protein, 51 aa, SARS-CoV-2
DVDLGDISGINASVVNIQKEIDRLNEVAKNLNESLIDLQELGKYEQYIKWP

SEQ ID NO: 10 - protein, 25 aa, SARS-CoV-2
PWYIWLGFIAGLIAIVMVTIMLCCM

SEQ ID NO: 11 - protein, 37 aa, SARS-CoV-2
MTSCCSCLKGCCSCGSCCKFDEDDSEPVLKGVKLHYT

SEQ ID NO: 12 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 13 - protein, 137 aa, SARS-CoV-2
LPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQ

SEQ ID NO: 14 - protein, 118 aa, SARS-CoV-2
KKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPP

SEQ ID NO: 15 - protein, 75 aa, SARS-CoV-2
MYSFVSEETGTLIVNSVLLFLAFVVFLLVTLAILTALRLCAYCCNIVNVSLVKPSFYVYSRVKNLNSSRVPDLLV

SEQ ID NO: 16 - protein, 7 aa, SARS-CoV-2
MYSFVSE

SEQ ID NO: 17 - protein, 31 aa, SARS-CoV-2
ETGTLIVNSVLLFLAFVVFLLVTLAILTALR

SEQ ID NO: 18 - protein, 37 aa, SARS-CoV-2
LCAYCCNIVNVSLVKPSFYVYSRVKNLNSSRVPDLLV

SEQ ID NO: 19 - protein, 9 aa, SARS-CoV-2
QIAPGQTGK

SEQ ID NO: 20 - protein, 11 aa, Homo sapiens
ITLLSALVETR

SEQ ID NO: 21 - protein, 7 aa, Homo sapiens
LINDYVK

SEQ ID NO: 22 - protein, 7 aa, Homo sapiens
GPTQEFK

SEQ ID NO: 23 - protein, 11 aa, Homo sapiens
VGFYESDVMGR

SEQ ID NO: 24 - protein, 13 aa, Homo sapiens
LGNQEPGGQTALK

SEQ ID NO: 25 - protein, 12 aa, Homo sapiens
ALQDQLVLVAAK

SEQ ID NO: 26 - protein, 12 aa, Homo sapiens
DPTFIPAPIQAK

SEQ ID NO: 27 - protein, 9 aa, Homo sapiens
LSPLGEEMR

SEQ ID NO: 28 - protein, 16 aa, Homo sapiens
DALSSVQESQVAQQAR

SEQ ID NO: 29 - protein, 7 aa, Homo sapiens
AVEVLPK

SEQ ID NO: 30 - protein, 11 aa, Homo sapiens
GASQAGAPQGR

SEQ ID NO: 31 - protein, 10 aa, Homo sapiens
LDAQASFLPK

SEQ ID NO: 32 - protein, 7 aa, Homo sapiens
VTYDVSR

SEQ ID NO: 33 - protein, 9 aa, Homo sapiens
GPGGVWAAK

SEQ ID NO: 34 - protein, 9 aa, Homo sapiens
TLEAQLTPR

SEQ ID NO: 35 - protein, 7 aa, Homo sapiens
VSMMQTK

SEQ ID NO: 36 - protein, 13 aa, Homo sapiens
AADDTWEPFASGK

SEQ ID NO: 37 - protein, 9 aa, Homo sapiens
AGLQFPVGR

SEQ ID NO: 38 - protein, 9 aa, Homo sapiens
LLLPGELAK

SEQ ID NO: 39 - protein, 9 aa, Homo sapiens
LELSVDGAK

SEQ ID NO: 40 - protein, 10 aa, Homo sapiens
VGDTLNLNLR

SEQ ID NO: 41 - protein, 12 aa, Homo sapiens
LSLEIEQLELQR

SEQ ID NO: 42 - protein, 8 aa, Homo sapiens
TWYPEVPK

SEQ ID NO: 43 - protein, 7 aa, Homo sapiens
AEDLVGK

SEQ ID NO: 44 - protein, 10 aa, Homo sapiens
SLGFCDTTNK

SEQ ID NO: 45 - protein, 9 aa, Homo sapiens
FQSVFTVTR

SEQ ID NO: 46 - protein, 9 aa, Homo sapiens
GNLCVNLMR

SEQ ID NO: 47 - protein, 8 aa, Homo sapiens
GIYGTISR

SEQ ID NO: 48 - protein, 7 aa, Homo sapiens
VFQFLEK

SEQ ID NO: 49 - protein, 15 aa, Homo sapiens
SLPVSDSVLSGFEQR

SEQ ID NO: 50 - protein, 11 aa, Homo sapiens
LSPIYNLVPVK

SEQ ID NO: 51 - protein, 12 aa, Homo sapiens
FEDGVLDPDYPR

SEQ ID NO: 52 - protein, 16 aa, Homo sapiens
IFFYDSENPPASEVLR

SEQ ID NO: 53 - protein, 10 aa, Homo sapiens
IQNILTEEPK

SEQ ID NO: 54 - protein, 17 aa, Homo sapiens
LFDSDPITVTVPVEVSR

SEQ ID NO: 55 - protein, 11 aa, Homo sapiens
LVVLGSGGVGK

SEQ ID NO: 56 - protein, 12 aa, Homo sapiens
GPSVFPLAPSSK

SEQ ID NO: 57 - protein, 8 aa, Homo sapiens
GLPAPIEK

SEQ ID NO: 58 - protein, 12 aa, Homo sapiens
TPLGDTTHTCPR

SEQ ID NO: 59 - protein, 9 aa, Homo sapiens
TPLTATLSK

SEQ ID NO: 60 - protein, 7 aa, Homo sapiens
GFPSVLR

SEQ ID NO: 61 - protein, 8 aa, Homo sapiens
GLPSSIEK

SEQ ID NO: 62 - protein, 9 aa, Homo sapiens
YTCLPGYVR

SEQ ID NO: 63 - protein, 9 aa, Homo sapiens
DISEVVTPR

SEQ ID NO: 64 - protein, 9 aa, Homo sapiens
MESLGITSR

SEQ ID NO: 65 - protein, 10 aa, Homo sapiens
NQVSLTCLVK

SEQ ID NO: 66 - protein, 20 aa, Homo sapiens
VDNALQSGNSQESVTEQDSK

SEQ ID NO: 67 - protein, 15 aa, Homo sapiens
YAASSYLSLTPEQWK

SEQ ID NO: 68 - protein, 7 aa, SARS-CoV2
GVYYPDK

SEQ ID NO: 69 - protein, 9 aa, SARS-CoV2
FASVYAWNR

SEQ ID NO: 70 - protein, 10 aa, SARS-CoV2
VGGNYNYLYR

SEQ ID NO: 71 - protein, 9 aa, SARS-CoV2
QIAPGQTGK